SEQUENCE LISTING (8 sequences)

SEQ ID NO: 1 - protein, 122 aa, Artificial Sequence
  Description of Artificial Sequence Synthetic polypeptide
QVQLQQSGAEVKKPGASVKVSCKASGYTFTSYGISWVRQAPGQGLEWMGWISAYNGNTNYAQKLQGRVTMTTDTSTSTAYMELRSLRSDDTAVYYCARNGLWGWDSDAFDIWGRGTLVTVSS

SEQ ID NO: 2 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTVVFGGGTKVTVL

SEQ ID NO: 3 - protein, 5 aa, Artificial Sequence
  Description of Artificial Sequence Synthetic peptide
SYGIS

SEQ ID NO: 4 - protein, 17 aa, Artificial Sequence
  Description of Artificial Sequence Synthetic peptide
WISAYNGNTNYAQKLQG

SEQ ID NO: 5 - protein, 13 aa, Artificial Sequence
  Description of Artificial Sequence Synthetic peptide
NGLWGWDSDAFDI

SEQ ID NO: 6 - protein, 14 aa, Artificial Sequence
  Description of Artificial Sequence Synthetic peptide
TGTSSDVGGYNYVS

SEQ ID NO: 7 - protein, 7 aa, Artificial Sequence
  Description of Artificial Sequence Synthetic peptide
DVSNRPS

SEQ ID NO: 8 - protein, 10 aa, Artificial Sequence
  Description of Artificial Sequence Synthetic peptide
SSYTSSSTVV

(19) United States
(12) Patent Application Publication
(10) Pub. No.: US 2022/0403020 A1
(43) Pub. Date: Dec. 22, 2022

(54) METHODS OF TREATMENT USING ILT7 BINDING PROTEINS

(71) Applicant: Viela Bio, Inc., Gaithersburg, MD (US)

(72) Inventors: William REES, North Bethesda, MD (US); John N. RATCHFORD, Lutherville, MD (US); Jodi KARNELL, Gaithersburg, MD (US); Jorn DRAPPA, Gaithersburg, MD (US); Gabor ILLEI, Gaithersburg, MD (US)

(21) Appl. No.: 17/831,784

(22) Filed: Jun. 3, 2022

Related U.S. Application Data
(63) Continuation of application No. PCT/US2020/063396, filed on Dec. 4, 2020.
(60) Provisional application No. 63/109,923, filed on Nov. 5, 2020.
(60) Provisional application No. 63/083,649, filed on Sep. 25, 2020.
(60) Provisional application No. 63/024,182, filed on May 13, 2020.
(60) Provisional application No. 63/023,820, filed on May 12, 2020.
(60) Provisional application No. 62/944,845, filed on Dec. 6, 2019.

(51) Int. Cl. C07K 16/24; C07K 16/28; C12Q 1/6809; A61P 37/06

(52) U.S. Cl. CPC C07K 16/249; C07K 16/2866; C12Q 1/6809; A61P 37/06; C12Q 2600/112; C07K 2317/565; A61K 2039/505

(57) ABSTRACT

The present disclosure is related to methods of treating autoimmune disorders in a subject comprising administering immunoglobulin-like transcript 7 (ILT7) binding proteins to a subject having elevated type I interferon gene signature (IFNGS). The present disclosure also relates to methods of reducing pDCs in tissues comprising administering an ILT7-binding protein to a subject in need thereof.

### CROSS-REFERENCE TO RELATED APPLICATIONS

This application is a continuation of International Application No. PCT/US2020/063396, filed Dec. 4, 2020, which claims the benefit of priority to U.S. Provisional Application No. 62/944,845, filed Dec. 6, 2019, U.S. Provisional Application No. 63/023,820, filed May 12, 2020, U.S. Provisional Application No. 63/024,182, filed May 13, 2020, U.S. Provisional Application No. 63/083,649, filed Sep. 25, 2020, and U.S. Provisional Application No. 63/109,923, filed Nov. 5, 2020, each of which is entirely incorporated herein by reference for all purposes.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is HOPA-025_06_US_SeqList_ST25.txt. The text file is 3,961 bytes, was created on Jun. 1, 2022, and is being submitted electronically via EFS-Web.

### BACKGROUND

### Field

The present disclosure is related to methods of treating autoimmune disorders in a subject comprising administering immunoglobulin-like transcript 7 (ILT7) binding proteins to a subject having elevated type I interferon gene signature (IFNGS). The present disclosure is also relates to methods of reducing pDCs in tissues comprising administering an ILT7-binding protein to a subject in need thereof.

### Background

The type I interferon (IFN) axis is one of the most significant pathways in human disease, and its dysregulation is central to the pathogenesis of many chronic autoimmune diseases, such as systemic lupus erythematosus (SLE). Although the precise etiology of SLE and other autoimmune diseases is not fully resolved, it is believed that a combination of environmental and genetic factors, together with an accumulation of cellular debris, leads to a breakdown in peripheral immune tolerance, characterized by high levels of circulating autoreactive antibodies. Currently available methods are directed towards treating autoimmune diseases and not towards preventing such diseases. Further, conventional treatment options for autoimmune diseases include immunosuppressant drugs that are associated with a wide range of side effects. Thus, there is a need for prophylactic and better therapeutic alternatives for treating and preventing autoimmune diseases. The present disclosure addresses these needs.

### SUMMARY

In certain embodiments, the methods of the present disclosure can be used for reducing a type I interferon gene signature (IFNGS) in a subject in need thereof. The methods comprise administering to the subject a pharmaceutically effective amount of an immunoglobulin-like transcript 7 (ILT7)-binding protein. The ILT7 binding protein is administered to the subject when the type I IFNGS is elevated in the subject relative to the type I IFNGS in a normal subject. In a specific embodiment, the ILT7 binding protein may be administered to subjects with elevated baseline type I IFNGS relative to the type I IFNGS in a normal subject, these subjects are monitored for reduction of the type I IFNGS after treatment. The ILT7-binding protein binds to the same ILT7 epitope as an antibody comprising a heavy chain variable region (VH) of SEQ ID NO:1 and a light chain variable region (VL) of SEQ ID NO:2. In certain aspects, the subject is monitored for reduction of the type I IFNGS after treatment.

In certain aspects, the type I IFNGS is measured in a test biological sample taken from the subject. The test sample includes, but is not limited to, blood, sputum, saliva, skin cells, skin biopsy samples, kidney cells, lung cells, liver cells, heart cells, brain cells, nervous tissue, thyroid cells, eye cells, skeletal muscle cells, cartilage, bone tissue, and cultured cells.

In some aspects, the type I IFNGS is elevated by at least about 4-fold in the test biological sample relative to the normal biological sample. In certain aspects, the type I IFNGS comprises the collective expression levels of two or more type I interferon (IFN)-inducible genes. In some aspects, the two or more type I interferon (IFN)-inducible genes are selected from the group consisting of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18. In certain aspects, the type I IFNGS comprises the collective expression levels of all of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18.

In some aspects, the type I IFNGS is determined by assaying the mRNA levels of the two or more type I interferon (IFN)-inducible genes in the test biological sample. In particular aspects, the type I IFNGS is determined by assaying the mRNA levels of the 21 type I interferon (IFN)-inducible genes in the test biological sample.

In some aspects, administering the ILT7-binding protein causes a reduction in plasmacytoid dendritic cells (pDCs) in the subject. In certain aspects, the pDCs are circulating pDCs. In particular aspects, the reduction in the pDCs is reversible.

In some aspects, reducing the type I IFNGS treats an autoimmune disease in the subject. In certain aspects, the autoimmune disease is selected from the group consisting of systemic lupus erythematosus (SLE), lupus nephritis, cutaneous lupus erythematosus (CLE), Sjögren's syndrome, inflammatory myositis, such as dermatomyositis, inclusion body myositis, juvenile myositis and polymyositis, systemic sclerosis, diabetes, Hashimoto's disease, autoimmune adrenal insufficiency, pure red cell anemia, multiple sclerosis, rheumatic carditis, psoriasis, psoriatic arthritis, rheumatoid arthritis, chronic inflammation, chronic rheumatism, vitiligo, alopecia areata, hidradenitis suppurativa, celiac disease, acute and chronic graft versus host disease (GVHD), vascular inflammation, myocardial infarction, and Type-1 interferonopathies. In some aspects, the autoimmune disease is SLE or CLE. In other aspects, the autoimmune disease is Sjögren's syndrome. In yet other aspects, the autoimmune disease is dermatomyositis. In other aspects, the autoimmune disease is polymyositis. In yet other aspects, the autoimmune disease is systemic sclerosis. In still other aspects, the autoimmune disease is hidradenitis suppurativa. In other aspects, the autoimmune disease is vitiligo.

In some aspects, the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively. In particular aspects, the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2. In certain aspects, the ILT7-binding protein is an antibody comprising a heavy chain variable region (VH) of SEQ ID NO:1 and a light chain variable region (VL) of SEQ ID NO:2. In some aspects, the antibody is afucosylated.

In some aspects, the pharmaceutically effective amount of the ILT7-binding protein ranges from about 0.1 mg to about 1000 mg. In certain aspects, the pharmaceutically effective amount of the ILT7-binding protein is about 1 mg, about 5 mg, about 15 mg, about 50 mg, about 100 mg, or about 150 mg. In some aspects, the ILT7-binding protein is administered by subcutaneous injection.

In some aspects, administration of the ILT7-binding protein leads to at least about 50% reduction in the type I IFNGS in the subject, compared to the type I IFNGS prior to administration of the ILT7-binding protein. In certain aspects, the ILT7-binding protein induces antibody-dependent cell-mediated cytotoxicity (ADCC) activity against pDCs. In some aspects, the ILT7-binding protein suppresses release of type I interferon (IFN) from pDCs. In certain aspects, the type I IFN is IFNα. In some aspects, the ILT7-binding protein specifically binds to ILT7. In some aspects, the ILT7 is located on pDCs.

In other embodiments, the methods of the present disclosure can be used to monitor the effectiveness of treatment of conditions marked by activated pDCs. The methods comprise the steps of: (a) measuring a type I interferon gene signature (IFNGS) in a biological sample taken from the subject to obtain a baseline value of the type I IFNGS; and (b) measuring the type I IFNGS in a biological sample taken from the subject after administering a treatment, wherein the treatment comprises an immunoglobulin-like transcript 7 (ILT7)-binding protein. In some aspects a decrease in the type I IFNGS in step (b) compared to the baseline value indicates that the treatment is effective. The ILT7-binding protein binds to the same ILT7 epitope as an antibody comprising a heavy chain variable region (VH) of SEQ ID NO:1 and a light chain variable region (VL) of SEQ ID NO:2.

In additional embodiments, the methods of the present disclosure can be used for reducing plasmacytoid dendritic cells (pDCs) in a tissue of a subject in need thereof. The methods comprise administering to the subject a pharmaceutically effective amount of an immunoglobulin-like transcript 7 (ILT7)-binding protein. In certain aspects, the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

In some aspects, the tissue is selected from the group consisting of skin cells, skin biopsy samples, kidney cells, lung cells, liver cells, heart cells, brain cells, nervous tissue, thyroid cells, eye cells, skeletal muscle cells, cartilage, bone tissue, and cells from airway passages. In certain aspects, the tissue is a skin cell. In some aspects, the tissue is a skin biopsy sample.

In some aspects, the method results in a decrease in pDCs in the tissue compared to a baseline value. In certain aspects, the decrease in pDCs in the tissue compared to the baseline value ranges from about 1% to about 99%. In some aspects, the decrease in pDCs in the tissue compared to the baseline value is at least about 50%.

In some aspects, the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

In further embodiments, the methods of the present disclosure can be used for treating an autoimmune disorder in a subject in need thereof. The methods comprise administering to the subject a pharmaceutically effective amount of an immunoglobulin-like transcript 7 (ILT7)-binding protein. In some aspects, the pharmaceutically effective amount of the ILT7-binding protein is about 1 mg, about 5 mg, about 15 mg, about 50 mg, about 100 mg, or about 150 mg. In some aspects, the pharmaceutically effective amount of the ILT7-binding protein is about 50 mg. In certain aspects, the pharmaceutically effective amount of the ILT7-binding protein is about 150 mg.

In certain embodiments, the methods of the present disclosure can be used for treating an autoimmune disorder in a subject in need thereof, the methods comprising administering to the subject a pharmaceutically effective amount of an immunoglobulin-like transcript 7 (ILT7)-binding protein, wherein the pharmaceutically effective amount of the ILT7-binding protein is about 50 mg.

In other embodiments, the methods of the present disclosure can be used for treating an autoimmune disorder in a subject in need thereof, the methods comprising administering to the subject a pharmaceutically effective amount of an immunoglobulin-like transcript 7 (ILT7)-binding protein, wherein the pharmaceutically effective amount of the ILT7-binding protein is about 150 mg.

In additional embodiments, the methods of the present disclosure can be used for reducing plasmacytoid dendritic cells (pDCs) in a tissue of a subject in need thereof, the method comprising administering to the subject a pharmaceutically effective amount of an immunoglobulin-like transcript 7 (ILT7)-binding protein. The pharmaceutically effective amount of the ILT7-binding protein is about 50 mg.

In further embodiments, the methods of the present disclosure can be used for reducing plasmacytoid dendritic cells (pDCs) in a tissue of a subject in need thereof, the method comprising administering to the subject a pharmaceutically effective amount of an immunoglobulin-like transcript 7 (ILT7)-binding protein. The pharmaceutically effective amount of the ILT7-binding protein is about 150 mg.

In some aspects, the decrease in pDCs in the tissue compared to the baseline value ranges from about 1% to about 99%. In certain aspects, the decrease in pDCs in the tissue compared to the baseline value is at least about 50%.

In some aspects, the subject has a high blood type I IFNGS level prior to administration of the ILT7-binding protein. In particular aspects, subject has a high pDC level in a tissue biopsy prior to administration of the ILT7-binding protein.

In some aspects, the autoimmune disease is systemic lupus erythematosus (SLE), lupus nephritis, cutaneous lupus erythematosus (CLE), Sjögren's syndrome, inflammatory myositis, such as dermatomyositis, inclusion body myositis, juvenile myositis and polymyositis, systemic sclerosis, diabetes, Hashimoto's disease, autoimmune adrenal insufficiency, pure red cell anemia, multiple sclerosis, rheumatic carditis, psoriasis, psoriatic arthritis, rheumatoid arthritis, chronic inflammation, chronic rheumatism, vitiligo, alopecia areata, hidradenitis suppurativa, celiac disease, acute and chronic graft versus host disease (GVHD), vascular inflammation, myocardial infarction, and Type-1 interferonopathies. In some aspects, the autoimmune disease is SLE. In other aspects, the autoimmune disease is CLE. In some aspects, the autoimmune disease is lupus. In certain aspects, the subject does not have discoid lupus erythematosus (DLE).

In some embodiments, the methods of the present disclosure can be used for selecting a patient for treatment with an ILT7-binding protein, the method comprising: (i) determining the baseline blood type I IFNGS level of the patient, and (ii) selecting those patients with high baseline blood type I IFNGS levels for treatment with the ILT7-binding protein.

In certain embodiments, the methods of the present disclosure are directed to treating an autoimmune disorder in a subject in need thereof, the method comprising administering to the subject a pharmaceutically effective amount of an immunoglobulin-like transcript 7 (ILT7)-binding protein, wherein the subject is determined to have a high blood type I IFNGS level prior to administration of the ILT7-binding protein. In some aspects, the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively. In some aspects, the ILT7 binding protein is an antibody comprising a variable heavy chain (VH) that is at least 85%, 90%, 95%, 96%, 97%, 98% or 99% identical to SEQ ID NO:1 and/or a variable light chain (VL) that is at least 85%, 90%, 95%, 96%, 97%, 98% or 99% identical to SEQ ID NO:2. In certain aspects, the ILT7-binding protein is an antibody comprising a heavy chain variable region (VH) of SEQ ID NO:1 and a light chain variable region (VL) of SEQ ID NO:2. In some aspects, the antibody is afucosylated.

### BRIEF DESCRIPTION OF THE FIGURES

FIG. 1 shows the overall study design for a Phase Ia, randomized, blinded, placebo-controlled study to evaluate the safety and tolerability of single-ascending subcutaneous doses of the ILT7-binding protein used in the methods described herein in subjects suffering from at least one of the following five autoimmune diseases: systemic lupus erythematosus (SLE), Sjögren's syndrome, dermatomyositis, polymyositis, or systemic sclerosis.

FIG. 2 shows details of the single ascending dose study design.

FIG. 3 shows the mean serum concentration profile of the ILT7-binding protein used in the methods described herein following a single subcutaneous dose in a subject suffering from at least one of the following five autoimmune diseases: SLE, Sjögren's syndrome, dermatomyositis, polymyositis, or systemic sclerosis.

FIG. 4 shows pDC levels (%) over time, as a percent of the baseline level (value using % peripheral blood mononuclear cells) in a subject, suffering from at least one of the following five autoimmune diseases: SLE, Sjögren's syndrome, dermatomyositis, polymyositis, or systemic sclerosis, following a single subcutaneous dose (1 mg, 5 mg, 15 mg, 50 mg, or 150 mg) of an ILT7-binding protein used in the methods described herein (VIB7734).

FIG. 5 shows pDC levels (%) over time, as a percent of the baseline level (value using absolute concentration) in a subject, suffering from at least one of the following five autoimmune diseases: SLE, Sjögren's syndrome, dermatomyositis, polymyositis, or systemic sclerosis, following a single subcutaneous dose (1 mg, 5 mg, 15 mg, 50 mg, or 150 mg) of an ILT7-binding protein used in the methods described herein (VIB7734).

FIG. 6 shows pDC levels (absolute concentration; cells/microliter) in a subject, suffering from at least one of the following five autoimmune diseases: SLE, Sjögren's syndrome, dermatomyositis, polymyositis, or systemic sclerosis, following a single subcutaneous dose (1 mg, 5 mg, 15 mg, 50 mg, or 150 mg) of an ILT7-binding protein used in the methods described herein (VIB7734).

FIG. 7A and FIG. 7B show type I IFNGS fold change (measured as % of baseline) in subjects with high IFN that are treated with 1-150 mg of an ILT7-binding protein used in the methods described herein (VIB7734). The type I IFNGS was determined by assaying the collective mRNA levels of 21 type I IFN-inducible genes in a biological sample taken from the subjects, determining an average value (mean or median) of the mRNA levels of 21 type I IFN-inducible gene, normalizing the average value against an average of mRNA levels of 3 housekeeping genes (18S rRNA, β actin, and Glyceraldehyde 3-phosphate dehydrogenase (GAPDH)), and obtaining a composite outcome. In a majority of subjects with a elevated type I IFNGS, reduction in pDC levels (FIG. 7A) correlated with a reduction in type I IFNGS (reported as % of baseline fold change) (FIG. 7B).

FIG. 8 shows that a type I IFNGS is reduced in subjects with elevated baseline type I IFNGS that are treated with 15 mg of an ILT7-binding protein used in the methods described herein (VIB7734), but not reduced in subjects with low baseline type I IFNGS. The type I IFNGS was determined by assaying the collective mRNA levels of 21 type I IFN-inducible genes in a biological sample taken from the subjects, determining an average value (mean or median) of the mRNA levels of 21 type I IFN-inducible gene, normalizing the average value against an average of mRNA levels of 3 housekeeping genes (18S rRNA, β actin, and GAPDH), and obtaining a composite outcome.

FIG. 9 shows the overall study design for a Phase Ib, randomized, blinded, placebo-controlled study to evaluate the safety and tolerability of multiple ascending subcutaneous doses of an ILT7-binding protein used in the methods described herein (VIB7734) in subjects with at least one of the following autoimmune diseases: systemic lupus erythematosus (SLE), cutaneous lupus erythematosus (CLE), systemic sclerosis, polymyositis, and dermatomyositis.

FIG. 10 shows the randomization and dose escalation scheme of the multiple ascending dose (MAD) study.

FIG. 11A and FIG. 11B show the serum concentration profile of an ILT7-binding protein used in the methods described herein (VIB7734) following multiple subcutaneous doses (every 4 weeks for 3 doses) of 5 mg (Cohort 1) or 50 mg (Cohort 2) of VIB7734. FIG. 11A shows the serum concentration profile of VIB7734 in Cohort 2 subjects. FIG. 11B shows the mean serum concentration profile of VIB7734 in subjects in Cohort 1 (solid circles) and Cohort 2 (solid squares).

FIG. 12A and FIG. 12B show pDC levels (%) over time, as a percent of the baseline level (value using % peripheral blood mononuclear cells) in whole blood of subjects in Cohort 1, suffering from at least one of the following autoimmune diseases: SLE, CLE, systemic sclerosis, polymyositis, and dermatomyositis, following multiple subcutaneous doses (every 4 weeks for 3 doses) of 5 mg of an ILT7-binding protein used in the methods described herein (VIB7734). Subjects in Cohort 1 were administered either a placebo (FIG. 12A) or VIB7734 (FIG. 12B).

FIG. 13A and FIG. 13B show pDC levels (%) over time, as a percent of the baseline level (value using absolute concentration) in whole blood of subjects in Cohort 1, suffering from at least one of the following autoimmune diseases: SLE, CLE, systemic sclerosis, polymyositis, and dermatomyositis, following multiple subcutaneous doses (every 4 weeks for 3 doses) of 5 mg of an ILT7-binding protein used in the methods described herein (VM7734). Subjects in Cohort 1 were administered either a placebo (FIG. 13A) or VIB7734 (FIG. 13B).

FIG. 14A and FIG. 14B show pDC levels (absolute concentration; cells/microliter) over time, in whole blood of subjects in Cohort 1, suffering from at least one of the following autoimmune diseases: SLE, CLE, systemic sclerosis, polymyositis, and dermatomyositis, following multiple subcutaneous doses (every 4 weeks for 3 doses) of 5 mg of an ILT7-binding protein used in the methods described herein (VIB7734). Subjects in Cohort 1 were administered either a placebo (FIG. 14A) or VIB7734 (FIG. 14B).

FIG. 15A-FIG. 15D show pDC levels (%) over time, as a percent of the baseline level (value using % peripheral blood mononuclear cells) in whole blood of subjects in Cohort 2 and Cohort 3, suffering from SLE or CLE, following multiple subcutaneous doses (every 4 weeks for 3 doses) of 50 mg (Cohort 2) or 150 mg (Cohort 3) of an ILT7-binding protein used in the methods described herein (VIB7734). Subjects in Cohort 2 were administered either a placebo (FIG. 15A) or VIB7734 (FIG. 15B). Subjects in Cohort 3 were administered either a placebo (FIG. 15C) or VIB7734 (FIG. 15D).

FIG. 16A-FIG. 16D show pDC levels (%) over time, as a percent of the baseline level (value using absolute concentration) in whole blood of subjects in Cohort 2 and Cohort 3 following multiple subcutaneous doses (every 4 weeks for 3 doses) of 50 mg (Cohort 2) or 150 mg (Cohort 3) of an ILT7-binding protein used in the methods described herein (VM7734). Subjects in Cohort 2 were administered either a placebo (FIG. 16A) or VIB7734 (FIG. 16B). Subjects in Cohort 3 were administered either a placebo (FIG. 16C) or VIB7734 (FIG. 16D).

FIG. 17A-FIG. 17D show_pDC levels (absolute concentration; cells/microliter) over time in whole blood of subjects in Cohort 2 and Cohort 3 following multiple subcutaneous doses (every 4 weeks for 3 doses) of 50 mg (Cohort 2) or 150 mg (Cohort 3) of an ILT7-binding protein used in the methods described herein (VIB7734). Subjects in Cohort 2 were administered either a placebo (FIG. 17A) or VIB7734 (FIG. 17B). Subjects in Cohort 3 were administered either a placebo (FIG. 17C) or VIB7734 (FIG. 17D).

FIG. 18A-FIG. 18D show_pDC levels over time, as a percent of peripheral blood mononuclear cells in whole blood of subjects in Cohort 2 and Cohort 3 following multiple subcutaneous doses (every 4 weeks for 3 doses) of 50 mg (Cohort 2) or 150 mg (Cohort 3) of an ILT7-binding protein used in the methods described herein (VIB7734). Subjects in Cohort 2 were administered either a placebo (FIG. 18A) or VIB7734 (FIG. 18B). Subjects in Cohort 3 were administered either a placebo (FIG. 18C) or VIB7734 (FIG. 18D).

FIG. 19A-FIG. 19D show median of pDC levels over time in whole blood of subjects in Cohort 2 following multiple subcutaneous doses (every 4 weeks for 3 doses) of 50 mg of an ILT7-binding protein used in the methods described herein (VIB7734) or a placebo. FIG. 19A: median of absolute pDC levels in blood over time, as a percent of the baseline level (value using absolute concentration). FIG. 19B: median of pDC levels (%) in blood over time, as a percent of the baseline level (value using % peripheral blood mononuclear cells (PBMCs)). FIG. 19C: median of pDC levels in blood over time, as a percent of PBMCs. FIG. 19D: median of absolute pDC levels (cells/μL) in blood over time.

FIG. 20A-FIG. 20D show median of pDC levels over time in whole blood of subjects in Cohort 3 following multiple subcutaneous doses (every 4 weeks for 3 doses) of 150 mg of an ILT7-binding protein used in the methods described herein (VIB7734) or a placebo. FIG. 20A: median of absolute pDC levels in blood over time, as a percent of the baseline level (value using absolute concentration). FIG. 20B: median of pDC levels (%) in blood over time, as a percent of the baseline level (value using % PBMCs). FIG. 20C: median of pDC levels (%) in blood over time, as a percent of PBMCs. FIG. 20D: median of absolute pDC levels (cells/μL) in blood over time.

FIG. 21A-FIG. 21D show type I IFNGS levels (measured as fold change (FIGS. 21A and 21B) or absolute score (FIGS. 21C and 21D)) over time in whole blood of subjects in Cohort 2 treated with 50 mg of an ILT7-binding protein used in the methods described herein (VM7734). The type I IFNGS score was determined by assaying the collective mRNA levels of 21 type I IFN-inducible genes in blood taken from the subjects, determining an average value (mean or median) of the mRNA levels of 21 type I IFN-inducible gene, normalizing the average value against an average of mRNA levels of 3 housekeeping genes (18S rRNA, β actin, and Glyceraldehyde 3-phosphate dehydrogenase (GAPDH)), and obtaining a composite outcome. Subjects in Cohort 2 were administered either VIB7734 (FIGS. 21A and 21C) or a placebo (FIGS. 21B and 21D).

FIG. 22A-FIG. 22C show_median of type I IFNGS levels over time in whole blood of subjects in Cohort 2 following multiple subcutaneous doses (every 4 weeks for 3 doses) of 50 mg of an ILT7-binding protein used in the methods described herein (VIB7734) or a placebo. FIG. 22A: median of type I IFNGS levels (measured as fold change) in blood over time. FIG. 22B: median of type I IFNGS levels (measured as neutralization ratio) in blood over time. FIG. 22C: median of type I IFNGS levels (measured as absolute score) in blood over time.

FIG. 23A-FIG. 23B show CLASI-Activity (CLASI-A) score (measured as change from baseline) over time in subjects in Cohort 2 following multiple subcutaneous doses (every 4 weeks for 3 doses) of 50 mg of an ILT7-binding protein used in the methods described herein (VM7734). Subjects in Cohort 2 were administered either a placebo (FIG. 23A) or VIB7734 (FIG. 23B).

FIG. 24A-FIG. 24D show CLASI-A score (measured as individual plots) over time in subjects in Cohort 2 and Cohort 3 following multiple subcutaneous doses (every 4 weeks for 3 doses) of 50 mg (Cohort 2) or 150 mg (Cohort 3) of an ILT7-binding protein used in the methods described herein (VIB7734). Subjects in Cohort 2 were administered either VIB7734 (FIG. 24A) or a placebo (FIG. 24B). Subjects in Cohort 3 were administered either VIB7734 (FIG. 24C) or a placebo (FIG. 24D).

FIG. 25 shows CLASI-A score (measured as proportion of subjects with an at least 4 point reduction from baseline) over time in subjects in Cohort 2 following multiple subcutaneous doses (every 4 weeks for 3 doses) of 50 mg of an ILT7-binding protein used in the methods described herein (VIB7734), in comparison to subjects administered a placebo.

FIG. 26 shows CLASI-A score (measured as proportion of subjects with an at least 4 point reduction from baseline) over time in subjects in Cohort 3 following multiple subcutaneous doses (every 4 weeks for 3 doses) of 150 mg of an ILT7-binding protein used in the methods described herein (VIB7734), in comparison to subjects administered a placebo.

FIG. 27 shows CLASI-A score (measured as proportion of subjects with an at least 4 point reduction from baseline) over time in subjects in Cohorts 2 and 3 combined, following multiple subcutaneous doses (every 4 weeks for 3 doses) of 50 mg (Cohort 2) or 150 mg (Cohort 3) of an ILT7-binding protein used in the methods described herein (VIB7734), in comparison to subjects in Cohort 2 and 3 administered a placebo.

FIG. 28 shows the proportion of CLASI-A score responders in Cohorts 2 and 3 following multiple subcutaneous doses (every 4 weeks for 3 doses) of 50 mg (Cohort 2) or 150 mg (Cohort 3) of an ILT7-binding protein used in the methods described herein (VM7734), in comparison to subjects in Cohort 2 and 3 administered a placebo. Comparative data is shown for all subjects, subjects with discoid lupus erythematosus (DLE), and subjects without DLE.

FIG. 29 shows CLASI-A score (measured as proportion of subjects with an at least 50% reduction from baseline) over time in subjects in Cohort 2 following multiple subcutaneous doses (every 4 weeks for 3 doses) of 50 mg of an ILT7-binding protein used in the methods described herein (VIB7734), in comparison to subjects administered a placebo.

FIG. 30A-FIG. 30C show a comparison of CLASI-A score (FIG. 30A), absolute pDC blood levels (measured as a percent of the baseline level) (FIG. 30B) and type I IFNGS levels (measured as absolute score) (FIG. 30C) over time in subjects in Cohort 2 following multiple subcutaneous doses (every 4 weeks for 3 doses) of 50 mg of an ILT7-binding protein used in the methods described herein (VIB7734).

FIG. 31A-FIG. 31C show a comparison of CLASI-A score (FIG. 31A), absolute pDC blood levels (measured as a percent of the baseline level) (FIG. 31B) and type I IFNGS levels (measured as absolute score) (FIG. 31C) over time in subjects in Cohort 2 following multiple subcutaneous doses (every 4 weeks for 3 doses) of a placebo.

FIG. 32 shows a summary of subject level exploratory data synthesis.

FIG. 33A-FIG. 33C show median of CLASI-A score over time in subjects in Cohorts 2 and 3 administered an ILT7-binding protein used in the methods described herein (VIB7734) (solid circles) in comparison to subjects in Cohorts 2 and 3, administered a placebo (solid triangles). FIG. 33A: subjects in Cohort 2 were administered multiple subcutaneous doses (every 4 weeks for 3 doses) of 50 mg of VIB7734 or a placebo. FIG. 33B: subjects in Cohort 3 were administered multiple subcutaneous doses (every 4 weeks for 3 doses) of 150 mg of VIB7734 or a placebo. The median change in CLASI-A score from baseline at day 85 was unexpectedly higher for Cohort 3 subjects (−9.5 in the 150 mg VIB7734-treated group compared to −5 in the placebo-treated group) compared to that for Cohort 2 subjects (−5 in the 50 mg VIB7734-treated group compared to −2.5 in the placebo-treated group). For Cohort 2 subjects, the Least Squares mean difference between the VIB7734 and placebo arm at Day 85 was 0.14; 95% Cl (−9.86, 10.14, p=0.977). For Cohort 3 subjects, the Least Squares mean difference between the VIB7734 and placebo arm at Day 85 was −5.12; 95% Cl (−11.49, 1.24, p=0.108). FIG. 33C: percentage change from baseline (BL) in median CLASI-A score by treatment arm and visit for subjects in Cohort 2 and Cohort 3.

FIG. 34A-FIG. 34B show absolute biopsy pDC count (measured as number of cells per square mm) over time in skin biopsies of subjects in Cohort 2 following multiple subcutaneous doses (every 4 weeks for 3 doses) of 50 mg of an ILT7-binding protein used in the methods described herein (VIB7734) (FIG. 34B) or a placebo (FIG. 34A).

FIG. 35A-FIG. 35B show median of biopsy pDC count over time in skin biopsies of subjects in Cohort 2 following multiple subcutaneous doses (every 4 weeks for 3 doses) of 50 mg of an ILT7-binding protein used in the methods described herein (VIB7734) (solid circles) or a placebo (solid triangles). FIG. 35A: median of skin biopsy pDC count (measured as a percent of Day 1 baseline) of Cohort 2 subjects. FIG. 35B: median of skin biopsy pDC count (measured as number of cells per square mm) of Cohort 2 subjects. The median reduction of change in skin biopsy pDC count on Day 85 (measured as a percent of Day 1 baseline as well as number of cells per square mm) was 87% for VIB7734-treated Cohort 2 subjects compared to 47% for Cohort 2 subjects treated with a placebo.

FIG. 36A-FIG. 36B show biopsy Myxovirus protein A (MxA) (Pos % of ROI area) over time in skin biopsies of subjects in Cohort 2 following multiple subcutaneous doses (every 4 weeks for 3 doses) of 50 mg of an ILT7-binding protein used in the methods described herein (VIB7734) (FIG. 36B) or a placebo (FIG. 36A).

FIG. 37 shows median of biopsy MxA (measured as percent area positive for MxA; Pos % of ROI area) over time in skin biopsies of subjects in Cohort 2 following multiple subcutaneous doses (every 4 weeks for 3 doses) of 50 mg of an ILT7-binding protein used in the methods described herein (VIB7734) (solid circles) or a placebo (solid triangles).

FIG. 38A-FIG. 38E show a comparison of CLASI-A score (FIG. 38A), absolute pDC blood levels (measured as cells/μL) (FIG. 38B), blood type I IFNGS levels (measured as absolute score) (FIG. 38C), skin biopsy pDC count (measured as number of cells per square mm) (FIG. 38D), and blood normalized type I IFNGS levels (measured as fold change) (FIG. 38E) over time in subjects in Cohort 2 following multiple subcutaneous doses (every 4 weeks for 3 doses) of 50 mg of an ILT7-binding protein used in the methods described herein (VIB7734).

FIG. 39A-FIG. 39E show a comparison of CLASI-A score (FIG. 39A), absolute pDC blood levels (measured as cells/μL) (FIG. 39B), blood type I IFNGS levels (measured as absolute score) (FIG. 39C), skin biopsy pDC count (measured as number of cells per square mm) (FIG. 39D), and blood normalized type I IFNGS levels (measured as fold change) (FIG. 39E) over time in subjects in Cohort 2 following multiple subcutaneous doses (every 4 weeks for 3 doses) of a placebo.

FIG. 40 provides a summary of adverse effects (AE) observed in subjects in Cohorts 1, 2, and 3 following multiple subcutaneous doses (every 4 weeks for 3 doses) of 5 mg (Cohort 1), 50 mg (Cohort 2) or 150 mg (Cohort 3) of an ILT7-binding protein used in the methods described herein (VIB7734) or a placebo.

FIG. 41 provides a summary of adverse effects of special interest (AESI) observed in subjects in Cohorts 1, 2, and 3 following multiple subcutaneous doses (every 4 weeks for 3 doses) of 5 mg (Cohort 1), 50 mg (Cohort 2) or 150 mg (Cohort 3) of an ILT7-binding protein used in the methods described herein (VIB7734) or a placebo.

FIG. 42 provides an overview of the skin biopsy immunohistochemistry (IHC) analysis method used herein for subjects in Cohort 2 and Cohort 3. Two 4 mm punch biopsies were collected from the skin of each Cohort 2 subject, a baseline biopsy at visit 2 (Day 1) and a repeat biopsy at visit 11 (Day 85). The biopsies were sectioned longitudinally. Each panel was analyzed on three sections per biopsy (left, center and right). Three rounds of staining was performed to assess pDCs (BDCA+/ILT7+ cells), IFN activity (MxA+ pixels) and inflammatory infiltrate (CD45+ cells).

FIG. 43A-FIG. 43C show the analysis strategy for quantification of pDCs and CD45+ cells using the skin biopsy IHC analysis method for subjects in Cohort 2 and Cohort 3. 500 microns of dermis proximal to the dermal-epidermal junction (DEJ) (red outline, FIG. 43A) was used as the analysis region of pDCs and CD45+ cells. Any protrusions of epidermis were excluded from analysis region (FIG. 43A). The number of pDCs (BDCA+/ILT7+ cells) and CD45+ cells per square mm were measured as the readout. Consistent RGB values were used to identify positive cells (except in very few instances where background staining impacted detection with those settings). FIG. 43B shows an analysis region without positive cell detection algorithm. FIG. 43C shows an analysis region with positive cell detection algorithm. Red indicates a positive cells; blue indicates negative.

FIG. 44A-FIG. 44C show the analysis strategy for quantification of Myxovirus protein A (MxA) for interferon (IFN) activity using the skin biopsy IHC analysis method for subjects in Cohort 2 and Cohort 3. The entire length of the epidermis (red outline, FIG. 44A) was used as the analysis region for MxA. The percent of area positive for MxA (% ROI MxA+) was measured as the readout. Consistent RGB values were used to identify positive pixels. FIG. 44B shows an analysis region without positive pixel detection algorithm. FIG. 44C shows an analysis region with positive pixel detection algorithm. Red indicates a positive pixels; blue indicates negative.

FIG. 45A-FIG. 45I show that there was minimal intra-biopsy variability in the baseline numbers of pDCs (BDCA+/ILT7+ cells), MxA+ pixels and CD45+ cells for each subject in Cohort 2. A high degree of consistency was observed within each skin biopsy at baseline. FIG. 45A: pDCs (BDCA+/ILT7+ cells) from center section of skin biopsy. FIG. 45B: MxA+ pixels from center section of skin biopsy. FIG. 45C: CD45+ cells from center section of skin biopsy. FIG. 45D: pDCs (BDCA+/ILT7+ cells) from right section of skin biopsy. FIG. 45E: MxA+ pixels from right section of skin biopsy. FIG. 45F: CD45+ cells from right section of skin biopsy. FIG. 45G: baseline pDCs (measured as number of cells per square mm) in skin biopsy from each subject in Cohort 2. FIG. 45H: baseline MxA+ pixels (measured as % ROI MxA+) in skin biopsy from each subject in Cohort 2. FIG. 45I: baseline CD45+ cells (measured as number of cells per square mm) in skin biopsy from each subject in Cohort 2. Each dot on the graphs (FIGS. 45G-I) represents an individual section (2-3 sections analyzed per biopsy).

FIG. 46A-FIG. 46L show that there was significant inter-biopsy variability in the baseline numbers of pDCs, MxA+ pixels and CD45+ cells within the subjects in Cohort 2. A high degree of variability was observed between skin biopsies at baseline. FIG. 46A: high pDCs (BDCA+/ILT7+ cells) from skin biopsy. FIG. 46B: high MxA+ pixels from skin biopsy. FIG. 46C: high CD45+ cells from skin biopsy. FIG. 46D: medium pDCs (BDCA+/ILT7+ cells) from skin biopsy. FIG. 46E: medium MxA+ pixels from skin biopsy. FIG. 46F: medium CD45+ cells from skin biopsy. FIG. 46G: low pDCs (BDCA+/ILT7+ cells) from skin biopsy. FIG. 46H: low MxA+ pixels from skin biopsy. FIG. 46I: low CD45+ cells from skin biopsy. FIG. 46J: baseline pDCs (measured as number of cells per square mm) in skin biopsy from each subject in Cohort 2. The subjects show high baseline pDCs (n=5, >100 pDCs/mm²), medium baseline pDCs (n=3, 10-100 pDCs/mm²), or low baseline pDCs (n=4, <10 pDCs/mm²). FIG. 46K: baseline MxA+ pixels (measured as % ROI MxA+) in skin biopsy from each subject in Cohort 2. The subjects show high baseline MxA+ pixels (n=5, >50% MxA+), medium baseline MxA+ pixels (n=4, 5-50% MxA+), or low baseline MxA+ pixels (n=2, <5% MxA+). FIG. 46L: baseline CD45+ cells (measured as number of cells per square mm) in skin biopsy from each subject in Cohort 2. The subjects show high baseline CD45+ cells (n=3, >2000 CD45+ cells/mm²), medium baseline CD45+ cells (n=4, 500-2000 CD45+ cells/mm²), or low baseline CD45+ cells (n=5, <500 CD45+ cells/mm²).

FIG. 47A-FIG. 47C show that while there was high variability of responses in reductions (measured by the percent change from baseline) in pDCs (FIG. 47A), MxA+ pixels (FIG. 47B), and CD45+ cells (FIG. 47C) in skin biopsies from Cohort 2 subjects treated with a placebo, more consistent reductions in pDCs, MxA+ pixels, and CD45+ cells were observed in skin biopsies from Cohort 2 subjects treated with an ILT7-binding protein used in the methods described herein (VIB7734).

FIG. 48A-FIG. 48C show that the skin biopsy IHC analysis method does not include threshold of activity. FIG. 48A: percent change from baseline of MxA in skin biopsies from Cohort 2 subjects treated with a placebo or an ILT7-binding protein used in the methods described herein (VIB7734). Gray outline indicates skin biopsy samples with substantial numerical fold increase in MxA. Overall, however, maintenance of very low levels of MxA was observed in the skin biopsy samples from Cohort 2 subjects. FIG. 48B: IHC performed on a skin biopsies from Cohort 2 subjects following multiple subcutaneous doses (every 4 weeks for 3 doses) of a placebo. FIG. 48C: IHC performed on skin biopsies from Cohort 2 subjects following multiple subcutaneous doses (every 4 weeks for 3 doses) of 50 mg of VIB7734.

FIG. 49 shows the relationship between high baseline pDC numbers/IFN activity and response to VIB7734 in skin biopsies of Cohort 2 subjects. VIB7734 treatment group: Cohort 2 subjects administered with multiple subcutaneous doses (every 4 weeks for 3 doses) of 50 mg of VIB7734, high baseline pDC numbers and high IFN activity was observed in skin biopsy in 4 of 5 responders. The non-responders had low baseline pDC or IFN activity in skin biopsy samples. Placebo group: Cohort 2 subjects administered with multiple subcutaneous doses (every 4 weeks for 3 doses) of a placebo showed no discernible relationship between pDCs or IFN activity and response.

FIG. 50 shows CLASI-A score (measured as proportion of subjects with an at least 7 point reduction from baseline) over time in subjects in Cohort 2 following multiple subcutaneous doses (every 4 weeks for 3 doses) of 50 mg of an ILT7-binding protein used in the methods described herein (VIB7734), in comparison to subjects administered a placebo.

FIG. 51 shows CLASI-A score (measured as proportion of subjects with an at least 7 point reduction from baseline) over time in subjects in Cohort 3 following multiple subcutaneous doses (every 4 weeks for 3 doses) of 150 mg of an ILT7-binding protein used in the methods described herein (VIB7734), in comparison to subjects administered a placebo.

FIG. 52 shows CLASI-A score (measured as proportion of subjects with an at least 7 point reduction from baseline) over time in subjects in Cohorts 2 and 3 combined, following multiple subcutaneous doses (every 4 weeks for 3 doses) of 50 mg (Cohort 2) or 150 mg (Cohort 3) of an ILT7-binding protein used in the methods described herein (VIB7734), in comparison to subjects in Cohort 2 and 3 administered a placebo.

FIG. 53 shows CLASI-A score (measured as proportion of subjects with an at least 50% reduction from baseline) over time in subjects in Cohort 3, following multiple subcutaneous doses (every 4 weeks for 3 doses) of 150 mg of an ILT7-binding protein used in the methods described herein (VIB7734), in comparison to subjects administered a placebo.

FIG. 54 shows CLASI-A score (measured as proportion of subjects with an at least 50% reduction from baseline) over time in subjects in Cohorts 2 and 3 combined, following multiple subcutaneous doses (every 4 weeks for 3 doses) of 50 mg (Cohort 2) or 150 mg (Cohort 3) of an ILT7-binding protein used in the methods described herein (VIB7734), in comparison to subjects in Cohort 2 and 3 administered a placebo.

FIG. 55A-FIG. 55C show normalized type I IFNGS levels (measured as fold change) over time in whole blood of subjects in Cohort 3 following multiple subcutaneous doses (every 4 weeks for 3 doses) of 150 mg of an ILT7-binding protein used in the methods described herein (VIB7734) or a placebo. FIG. 55A: normalized type I IFNGS levels over time in whole blood of subjects in Cohort 3 treated with VIB7734. FIG. 55B: normalized type I IFNGS levels over time in whole blood of subjects in Cohort 3 treated with a placebo. FIG. 55C: median of normalized type I IFNGS levels over time in whole blood of subjects in Cohort 3 treated with VIB7734 (solid circles) or placebo (solid triangles).

FIG. 56A-FIG. 56B show absolute biopsy pDC count (measured as number of cells per square mm) over time in skin biopsies of subjects in Cohort 3 following multiple subcutaneous doses (every 4 weeks for 3 doses) of 150 mg of an ILT7-binding protein used in the methods described herein (VIB7734) (FIG. 56B) or a placebo (FIG. 56A).

FIG. 57 shows median of biopsy pDC count (measured as number of cells per square mm) over time in skin biopsies of subjects in Cohort 3 following multiple subcutaneous doses (every 4 weeks for 3 doses) of 150 mg of an ILT7-binding protein used in the methods described herein (VIB7734) (solid circles) or a placebo (solid triangles). The median of skin biopsy pDC count on Day 85 (measured as a percent of Day 1 baseline) was reduced by 99% for subjects in Cohort 3 treated with VIB7734. In contrast, the median of skin biopsy pDC count on Day 85 (measured as a percent of Day 1 baseline) increased by 11% for subjects in Cohort 3 treated with placebo.

FIG. 58A-FIG. 58B show biopsy Myxovirus protein A (MxA) (Pos % of ROI area) over time in skin biopsies of subjects in Cohort 3 following multiple subcutaneous doses (every 4 weeks for 3 doses) of 150 mg of an ILT7-binding protein used in the methods described herein (VIB7734) (FIG. 58B) or a placebo (FIG. 58A).

FIG. 59 shows median of biopsy MxA (measured as percent area positive for MxA; median Pos % of ROI area) over time in skin biopsies of subjects in Cohort 3 following multiple subcutaneous doses (every 4 weeks for 3 doses) of 150 mg of an ILT7-binding protein used in the methods described herein (VIB7734) (solid circles) or a placebo (solid triangles). The median of skin biopsy MxA was reduced from a baseline value of 89.7% to 1.1% on Day 85 for subjects in Cohort 3 treated with VIB7734. In contrast, for placebo-treated Cohort 3 subjects, the median of skin biopsy MxA increased from a baseline value of 1.9% to 17.7% on Day 85.

FIG. 60A-FIG. 60B show absolute biopsy CD45 count (measured as number of CD45+ cells per square mm) over time in skin biopsies of subjects in Cohort 2 following multiple subcutaneous doses (every 4 weeks for 3 doses) of 50 mg of an ILT7-binding protein used in the methods described herein (VIB7734) (FIG. 60B) or a placebo (FIG. 60A).

FIG. 61 shows median of biopsy CD45 count (measured as number of CD45+ cells per square mm) over time in skin biopsies of subjects in Cohort 2 following multiple subcutaneous doses (every 4 weeks for 3 doses) of 50 mg of an ILT7-binding protein used in the methods described herein (VIB7734) (solid circles) or a placebo (solid triangles). The median of skin biopsy CD45 count was reduced from a baseline value of 1119 on Day 1 to 280 on Day 85 for subjects in Cohort 2 treated with VIB7734. In contrast, the median of skin biopsy CD45 count was reduced from a baseline value of 537 on Day 1 to 492 on Day 85 for subjects in Cohort 2 treated with placebo.

FIG. 62A-FIG. 62B show absolute biopsy CD45 count (measured as number of CD45+ cells per square mm) over time in skin biopsies of subjects in Cohort 3 following multiple subcutaneous doses (every 4 weeks for 3 doses) of 150 mg of an ILT7-binding protein used in the methods described herein (VIB7734) (FIG. 62B) or a placebo (FIG. 62A).

FIG. 63 shows median of biopsy CD45 count (measured as number of CD45+ cells per square mm) over time in skin biopsies of subjects in Cohort 3 following multiple subcutaneous doses (every 4 weeks for 3 doses) of 150 mg of an ILT7-binding protein used in the methods described herein (VIB7734) (solid circles) or a placebo (solid triangles). The median of skin biopsy CD45 count was reduced from a baseline value of 707 to 513 on Day 85 for subjects in Cohort 3 treated with VIB7734. In contrast, the skin biopsy CD45 count decreased from a baseline value of 897 to 666 on Day 85 for subjects in Cohort 3 treated with placebo.

FIG. 64A-FIG. 64D show a comparison of CLASI-A score (FIG. 64A), absolute pDC blood levels (measured as cells/μL) (FIG. 64B), blood normalized type I IFNGS levels (measured as fold change) (FIG. 64C) and skin biopsy pDC count (measured as number of cells per square mm) (FIG. 64D) over time for subjects in Cohort 3 following multiple subcutaneous doses (every 4 weeks for 3 doses) of 150 mg of an ILT7-binding protein used in the methods described herein (VIB7734).

FIG. 65A-FIG. 65D show a comparison of CLASI-A score (FIG. 65A), absolute pDC blood levels (measured as cells/μL) (FIG. 65B), blood normalized type I IFNGS levels (measured as fold change) (FIG. 65C) and skin biopsy pDC count (measured as number of cells per square mm) (FIG. 65D) over time in subjects in Cohort 3 following multiple subcutaneous doses (every 4 weeks for 3 doses) of a placebo.

FIG. 66A-FIG. 66B show that while pDCs (measured as percent change in number of cells from Day 1 baseline) in the skin were reduced in both Cohort 2 and Cohort 3 subjects treated with VIB7734 at 50 mg and 150 mg, respectively, the pDC depletion was more consistent for subjects in Cohort 3. FIG. 66A: Comparison of reduction of pDCs in skin biopsies of VIB7734-treated Cohort 2 and VIB7734-treated Cohort 3 subjects. The mean percent reduction of pDCs from baseline in skin samples with >10 pDCs/mm² at baseline was 96.31% for VIB7734-treated Cohort 3 subjects compared to 85.45% for VIB7734-treated Cohort 2 subjects. FIG. 66B: Comparison of reduction of MxA+ pixels (measured as percent change from Day 1 baseline) in skin biopsies of VIB7734-treated Cohort 2 and VIB7734-treated Cohort 3 subjects. The mean percent reduction of MxA+ pixels from Day 1 baseline in skin samples with >5% MxA+ at baseline was 76.84% for VIB7734-treated Cohort 3 subjects compared to 67.44% for VIB7734-treated Cohort 2 subjects.

FIG. 67A-FIG. 67B show the relationship between high baseline pDC numbers and response to VIB7734 in skin biopsies of Cohort 3 subjects. FIG. 67A: VIB7734 treatment group: Cohort 3 subjects administered with multiple subcutaneous doses (every 4 weeks for 3 doses) of 150 mg of VIB7734. FIG. 67B: Placebo group: Cohort 3 subjects administered with multiple subcutaneous doses (every 4 weeks for 3 doses) of a placebo. VIB7734 reduced levels of pDCs in the skin of Cohort 3 subjects.

FIG. 68A-FIG. 68B show CLASI-Activity (CLASI-A) score (measured as change from Day 1 baseline) over time in subjects in Cohort 3 following multiple subcutaneous doses (every 4 weeks for 3 doses) of 150 mg of an ILT7-binding protein used in the methods described herein (VIB7734). Subjects in Cohort 3 were administered either a placebo (FIG. 68A) or VIB7734 (FIG. 68B).

FIG. 69A-FIG. 69I show that there was minimal intra-biopsy variability in the baseline numbers of pDCs (BDCA+/ILT7+ cells), MxA+ pixels and CD45+ cells for each subject in Cohort 3. A high degree of consistency was observed within each skin biopsy at baseline. FIG. 69A: pDCs (BDCA+/ILT7+ cells) from center section of skin biopsy. FIG. 69B: MxA+ pixels from center section of skin biopsy. FIG. 69C: CD45+ cells from center section of skin biopsy. FIG. 69D: pDCs (BDCA+/ILT7+ cells) from right section of skin biopsy. FIG. 69E: MxA+ pixels from right section of skin biopsy. FIG. 69F: CD45+ cells from right section of skin biopsy. FIG. 69G: baseline pDCs (measured as number of cells per square mm) in skin biopsy from each subject in Cohort 3. FIG. 69H: baseline MxA+ pixels (measured as % ROI MxA+) in skin biopsy from each subject in Cohort 3. FIG. 69I: baseline CD45+ cells (measured as number of cells per square mm) in skin biopsy from each subject in Cohort 3. Each dot on the graphs (FIGS. 69G-I) represents an individual section (2-3 sections analyzed per biopsy).

FIG. 70A-FIG. 70L show that there was significant inter-biopsy variability in the baseline numbers of pDCs, MxA+ pixels and CD45+ cells within the subjects in Cohort 3. FIG. 70A: high pDCs (BDCA+/ILT7+ cells) from skin biopsy. FIG. 70B: high MxA+ pixels from skin biopsy. FIG. 70C: high CD45+ cells from skin biopsy. FIG. 70D: medium pDCs (BDCA+/ILT7+ cells) from skin biopsy. FIG. 70E: medium MxA+ pixels from skin biopsy. FIG. 70F: medium CD45+ cells from skin biopsy. FIG. 70G: low pDCs (BDCA+/ILT7+ cells) from skin biopsy. FIG. 70H: low MxA+ pixels from skin biopsy. FIG. 70I: low CD45+ cells from skin biopsy. A slightly increased baseline pDC and MxA signal was observed in subjects in Cohort 3 compared to subjects in Cohort 2. FIG. 70J: baseline pDCs (measured as number of cells per square mm) in skin biopsy from each subject in Cohort 2 and Cohort 3. The subjects in Cohort 2 show high baseline pDCs (n=5, >100 pDCs/mm²), medium baseline pDCs (n=3, 10-100 pDCs/mm²), or low baseline pDCs (n=4, <10 pDCs/mm²). The subjects in Cohort 3 show high baseline pDCs (n=5, >100 pDCs/mm²), medium baseline pDCs (n=3, 10-100 pDCs/mm²), or low baseline pDCs (n=2, <10 pDCs/mm²). FIG. 70K: baseline MxA+ pixels (measured as % ROI MxA+) in skin biopsy from each subject in Cohort 2 and Cohort 3. The subjects in Cohort 2 show high baseline MxA+ pixels (n=5, >50% MxA+), medium baseline MxA+ pixels (n=4, 5-50% MxA+), or low baseline MxA+ pixels (n=2, <5% MxA+). The subjects in Cohort 3 show high baseline MxA+ pixels (n=6, >50% MxA+) or low baseline MxA+ pixels (n=4, <5% MxA+). FIG. 70L: baseline CD45+ cells (measured as number of cells per square mm) in skin biopsy from each subject in Cohort 2 and Cohort 3. The subjects in Cohort 2 show high baseline CD45+ cells (n=3, >2000 CD45+ cells/mm²), medium baseline CD45+ cells (n=5, 500-2000 CD45+ cells/mm²), or low baseline CD45+ cells (n=4, <500 CD45+ cells/mm²). The subjects in Cohort 3 show high baseline CD45+ cells (n=2, >2000 CD45+ cells/mm²), medium baseline CD45+ cells (n=4, 500-2000 CD45+ cells/mm²), or low baseline CD45+ cells (n=4, <500 CD45+ cells/mm²).

FIG. 71A-FIG. 71C show that no clear impact of placebo was observed on biopsy markers at Day 85 for subjects in Cohort 3. FIG. 71A: pDC cells (measured as percent change from baseline). FIG. 71B: MxA+ pixels (measured as percent change from baseline). FIG. 71C: CD45+ cells (measured as percent change from baseline).

FIG. 72A-FIG. 72C show that for most subjects in Cohort 3 treated with 150 mg of an ILT7-binding protein used in the methods described herein (VM7734), a profound reduction in pDC cells (measured as percent change from baseline; FIG. 72A) and IFN activity (MxA+ pixels; measured as percent change from baseline; FIG. 72B) and a slight reduction in inflammatory infiltrate (CD45+ cells; measured as percent change from baseline; FIG. 72C) was observed at Day 85 compared to placebo-treated Cohort 3 subjects. FIG. 72A: for VIB7734-treated Cohort 3 subjects, a mean reduction of pDCs of 80.98+/−12.12 (mean+/−SEM) was observed compared to a mean increase of pDCs of 12.24+/−37.69 (mean+/−SEM) in placebo-treated Cohort 3 subjects. FIG. 72B: for VIB7734-treated Cohort 3 subjects, a mean reduction of MxA+ pixels of 58.29+/−17.88 (mean+/−SEM) was observed compared to a mean increase of MxA+ pixels of 773.6+/−866.33 (mean+/−SEM) in placebo-treated Cohort 3 subjects.

FIG. 73A-FIG. 73C show that for nearly all subjects in Cohort 3 (with a moderate or high signal at baseline) treated with 150 mg of an ILT7-binding protein used in the methods described herein (VIB7734), a profound reduction in pDC cells (measured as a percent of Day 1 baseline; FIG. 73A) and IFN activity (MxA+ pixels; measured as a percent of Day 1 baseline; FIG. 73B) and a reduction in inflammatory infiltrate (CD45+ cells; measured as a percent of Day 1 baseline; FIG. 73C) was observed at Day 85 compared to placebo-treated subjects in Cohort 3. Circles indicate samples from Cohort 3 subjects with low baseline activity.

FIG. 74A-FIG. 74C show the change in pDCs at Day 85 (d85) from baseline (BL) for each subject in Cohort 3 treated with 150 mg of an ILT7-binding protein used in the methods described herein (VIB7734) or a placebo. FIG. 74A: change in pDCs (measured as BDCA2+/ILT7+ cells) using the skin biopsy IHC analysis method for subjects in Cohort 3 (n=2; 10030044 and 10010052) treated with a placebo. FIG. 74B: change in pDCs (measured as BDCA2+/ILT7+ cells) using the skin biopsy IHC analysis method for subjects in Cohort 3 (n=8; 30010047, 10120061, 20070048, 200020040, 10010056, 10120055, 20060038, and 10140059) treated with 150 mg of VIB7734. FIG. 74C: change in pDCs (measured as number of cells per square mm) in skin biopsies of each of the VIB7734-treated Cohort 3 subjects and each of the placebo-treated Cohort 3 subjects at Day 85 compared to baseline.

FIG. 75A-FIG. 75D show combined data for subjects in Cohort 2 and Cohort 3 treated with 50 mg (Cohort 2) or 150 mg (Cohort 3) of an ILT7-binding protein used in the methods described herein (VIB7734). VIB7734 significantly reduces pDCs in the skin of subjects in Cohorts 2 and 3 treated with VIB7734 in comparison to subjects in Cohorts 2 and 3 treated with a placebo. FIG. 75A: changes in pDCs (measured as a percent of Day 1 baseline) for placebo-treated subjects in Cohorts 2 and 3 (n=6) compared to VIB7734-treated subjects in Cohorts 2 and 3 (n=16). The mean and median reductions in pDCs were 11.38% and 12.73%, respectively for placebo-treated subjects in Cohorts 2 and 3, compared to mean and median reductions in pDCs of 71.82% and 95.3%, respectively for VIB7734-treated subjects in Cohorts 2 and 3. FIG. 75B: changes in MxA+ pixels (measured as a percent of Day 1 baseline) for placebo-treated subjects in Cohorts 2 and 3 (n=6) compared to VIB7734-treated subjects in Cohorts 2 and 3 (n=16). The mean and median increase in MxA+ pixels were 269.3% and 38.8%, respectively for placebo-treated subjects in Cohorts 2 and 3, compared to mean and median reductions in pDCs of 52.9% and 84.48%, respectively for VIB7734-treated subjects in Cohorts 2 and 3. FIG. 75C-D: in all VIB7734-treated subjects with >2 pDCs/mm² in skin at baseline, correlations between percent change from baseline to day 85 in pDCs and MxA (FIG. 75C) or CD45+ cells (FIG. 75D) in skin biopsies were performed. Spearman correlations are shown.

FIG. 76A-FIG. 76D show that while pDCs (measured as number of cells per square mm) in the skin were reduced for both Cohort 2 and Cohort 3 subjects treated with VIB7734 at 50 mg and 150 mg, respectively, the pDC depletion was more consistent for subjects in Cohort 3. FIG. 76A: Reduction of pDCs in skin biopsies of all VIB7734-treated Cohort 2 subjects (n=8) at Day 85 compared to Day 1 baseline. FIG. 76B: Reduction of pDCs in skin biopsies of all VIB7734-treated Cohort 3 subjects (n=8) at Day 85 compared to Day 1 baseline. FIG. 76C: Reduction of pDCs in skin biopsies of VIB7734-treated Cohort 2 subjects without low baseline pDCs or IFN activity in skin biopsy samples (n=6) at Day 85 compared to Day 1 baseline. FIG. 76D: Reduction of pDCs in skin biopsies of VIB7734-treated Cohort 3 subjects without low baseline pDCs or IFN activity in skin biopsy samples (n=6) at Day 85 compared to Day 1 baseline.

FIG. 77 shows the median of circulating pDC levels (measured as % PBMC cells) in whole blood of subjects in Cohort 1, Cohort 2 and Cohort 3 treated with 5 mg (Cohort 1), 50 mg (Cohort 2), or 150 mg (Cohort 3) of the ILT7-binding protein used in the methods described herein (VIB7734). Reductions in median of circulating pDC levels were evident at week 1 and persisted through at least Day 85 in VIB7734-treated subjects in Cohort 1, Cohort 2 and Cohort 3, compared to the median of circulating pDC levels in placebo-treated subjects.

FIG. 78A-FIG. 78C show that the depletion of tissue-resident pDCs drives reductions in type-I IFN activity in the skin of subjects with cutaneous lupus. FIGS. 78A-C: MxA staining, defined as percent of region of interest area positive for MxA, was quantified in skin punch biopsies at baseline and study day 85. Each line represents an individual subject. Each point on the graph represents the mean percent MxA+ from serial sections within the biopsy.

FIG. 79A-FIG. 79C show that high baseline blood type I IFN activity is associated with higher rates of responsiveness to the ILT7-binding protein used in the methods described herein (VIB7734). FIG. 79A: percent change in whole blood type I IFNGS at the indicated time points is shown for placebo- and VIB7734-treated subjects (of cohorts 1, 2, and 3) with an elevated baseline type I IFNGS score (defined as 4-fold or higher relative to mean for healthy donors). The number of subjects with elevated baseline IFN activity is indicated for each group above the graph (cohort 1, n=3; cohort 2, n=6; cohort 3, n=8). Median and interquartile range are shown. FIG. 79B: percent change in serum IFNα levels is shown for placebo and VIB7734-treated subjects with elevated baseline IFNα levels (defined as two standard deviations above the healthy donor mean). Median and interquartile range are shown. FIG. 79C: correlation between baseline whole blood type I IFNGS and serum IFNα protein levels in VIB7734-treated subjects. Black dots indicate subjects classified as CLASI responders (defined as a 4-point or greater reduction in CLASI) and red dots indicate CLASI non-responders. The dotted lines represent a 4-fold change above the healthy donor mean (FC from HD mean) for type I IFNGS and 2 standard deviations above the healthy donor mean for IFNα protein.

### DETAILED DESCRIPTION OF THE INVENTION

Unless defined otherwise, all technical and scientific terms used herein have the same meaning as commonly understood by one of ordinary skill in the art to which the subject matter pertains. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

As used in this specification and the appended claims, the singular forms “a,” “an” and “the” include plural referents unless the context clearly dictates otherwise.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. The term “about” as used herein refers to a range that is 15% plus or minus from a stated numerical value within the context of the particular usage. For example, about 10 would include a range from 8.5 to 11.5. The term “about” also accounts for typical error or imprecision in measurement of values.

The disclosure provides methods for of treating an autoimmune disorder in a subject with an ILT7-binding protein. In certain aspects, the methods provide treating an autoimmune disorder in a subject in need thereof, wherein the subject is determined to have a high blood type I interferon gene signature (IFNGS) level. The disclosure also provides methods for reducing the IFNGS in a subject in need thereof. In some aspects, the methods comprise administering to the subject a pharmaceutically effective amount of an immunoglobulin-like transcript 7 (ILT7)-binding protein. In some aspects, the ILT7 binding protein is administered to the subject when the type I IFNGS is elevated in the subject relative to the type I IFNGS in a normal subject. In specific embodiments, for example, the ILT7 binding protein is administered to subjects with elevated baseline type I IFNGS relative to the type I IFNGS in a normal subject. In certain aspects, the methods provide selecting a patient for treatment with an ILT7-binding protein, the method comprising: (i) determining the baseline blood type I IFNGS level of the patient, and (ii) selecting those patients with high baseline blood type I IFNGS levels for treatment with the ILT7-binding protein. In particular aspects, the ILT7-binding protein is an antibody. In certain aspects, the antibody is VIB7734.

In certain embodiments, the type I IFNGS is a 21-gene signature. In some embodiments, the type I IFNGS in the subject is at elevated by at least 1.5-fold relative to a normal score prior to treatment. In some embodiments, the type I IFNGS in the subject is at elevated by at least 2-fold relative to a normal score prior to treatment. In certain embodiments, subjects with elevated type I IFNGS prior to treatment are more responsive to the treatment. In some embodiments, type I IFNGS is at least about 4-fold, at least about 5-fold, at least about 6-fold, at least about 7-fold, at least about 8-fold, at least about 9-fold, at least about 10-fold, at least about 11-fold, at least about 12-fold or higher relative to a normal score prior to treatment with an ILT7-binding protein used in the methods described herein. In particular embodiments, the tissue type I IFNGS is determined from a skin biopsy. In other embodiments, the tissue type I IFNGS is determined using the IFN-inducible Myxovirus protein A (MxA) immunohistochemistry (IHC) test. In further embodiments, the IFN-inducible gene expression in the epidermis is determined using skin tape stripping, RNA isolation and gene expression profiling (https://dermtech.com/wp-content/uploads/Lupus-Reference.pdf).

As used herein, the term “high” or “elevated” when used in conjunction with IFGNS means that the type I IFNGS is a fold change of at least about 1.1 to about 1000 compared to normal type I IFNGS. By “normal type I IFNGS” is intended a type I IFNGS obtained from a normal subject. The terms “high” or “elevated” when used in conjunction with type I IFNGS are used interchangeably. In some embodiments, the type I IFNGS is “high” or “elevated” when the type I IFNGS used in the methods described herein is at least about 1.1, 1.2, 1.3, 1.4, 1.5, 2, 3, 4, 5, 6, 7, 8, 9, 10, 15, 20, 30, 40, 50, 60, 70, 80, 90, or 100-fold relative to the type I IFNGS in a normal subject. In specific embodiments, the methods of treatment described herein are applied when type I IFNGS is elevated by at least about 4-fold relative to normal type I IFNGS.

In certain diseases (e.g., autoimmune diseases), activated pDCs secrete significant amounts of type I and type III interferons (IFNs). Type I IFNs are a large group of IFN proteins that help regulate the immune system. The mammalian IFNs are designated IFNα, IFNβ, IFNω, IFNε, IFNκ, IFNτ, IFNδ, IFNζ, and IFNν. In specific embodiments, the type I IFN that generates the type I IFNGS is IFNα. Type I IFN protein levels cannot be directly measured in a reliable way; however, measurement of IFN-inducible genes serves as a robust surrogate to Type 1 IFN protein levels. The expression levels of these type I IFN-inducible genes can be measured in biological samples (e.g., blood, skin, skeletal muscles, etc.) and analyzed as a composite outcome referred to as the “type I interferon gene signature” or “type I IFNGS” or “IFNGS.”

In certain embodiments, the type I IFNGS comprises expression levels of all type I IFN-inducible genes in a biological sample. In other embodiments, the type I IFNGS comprises expression levels of a subset of type I IFN-inducible genes in a biological sample.

In certain embodiments, the type I IFNGS is determined by assaying the expression levels of at least 2, at least 5, at least 10, at least 15, at least 20, at least 25, at least 30, at least 40, at least 50, at least 60, at least 70, at least 80, at least 90, at least 100, at least 200, at least 300, at least 400, or at least 500 type I IFN-inducible genes in a biological sample. In some embodiments, the type I IFNGS comprises the collective expression levels of two or more type I IFN-inducible genes. In certain embodiments, the two or more type I interferon (IFN)-inducible genes include, but are not limited to, two or more genes chosen from SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, or USP18. In certain cases, the type I IFNGS is determined by assaying the collective expression levels of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18. These gene symbols are well-known in the art and refer to human and non-human orthologs of the listed genes.

TABLE 1
Gene Symbols and Their Correlating Names.

| Gene | |
|---|---|
| Symbol | Correlating Name |
| SPATS2L | Spermatogenesis Associated Serine Rich 2 Like |
| EPSTI | Epithelial Stromal Interaction 1 |
| HERC5 | HECT and RLD Domain Containing E3 Ubiquitin Protein |
| | Ligase 5 |
| IFI27 | Interferon Alpha Inducible Protein 27 |
| IFI44 | Interferon Induced Protein 44 |
| IFI44L | Interferon Induced Protein 44-like |
| IFI6 | Interferon Alpha Inducible Protein 6 |
| IFIT1 | Interferon Induced Protein With Tetratricopeptide Repeats 1 |
| IFIT3 | Interferon Induced Protein With Tetratricopeptide Repeats 3 |
| ISG15 | Interferon Stimulated gene 15 |
| LAMP3 | Lysosomal Associated Membrane Protein 3 |
| LY6E | Lymphocyte Antigen 6 Family Member E |
| MX1 | MX Dynamin Like GTPase 1 |
| OAS1 | 2′-5′-Oligoadenylate Synthetase 1 |
| OAS2 | 2′-5′-Oligoadenylate Synthetase 2 |
| OAS3 | 2′-5′-Oligoadenylate Synthetase 3 |
| PLSCR1 | Phospholipid Scramblase 1 |
| RSAD2 | Radical S-Adenosyl Methionine Domain Containing 2 |
| RTP4 | Receptor Transporter Protein 4 |
| SIGLEC1 | Sialic Acid Binding Ig Like Lectin 1 |
| USP18 | Ubiquitin Specific Peptidase 18 |

In certain embodiments, the expression levels of the type I interferon (IFN)-inducible genes are determined by measuring the DNA levels (e.g., complementary DNA or cDNA levels) of the type I interferon (IFN)-inducible genes in a biological sample. In certain embodiments, the expression levels of the type I interferon (IFN)-inducible genes are determined by measuring the messenger RNA (mRNA) levels of the type I interferon (IFN)-inducible genes in a biological sample. In certain aspects, the type I IFNGS comprises mRNA levels of all type I IFN-inducible genes in the biological sample. In other aspects, the type I IFNGS comprises mRNA levels of a subset of type I IFN-inducible genes in the biological sample taken from a subject affected, likely to be affected, or suspected to be affected with a disease, e.g., an autoimmune disease. In certain aspects, the type I IFNGS is determined by assaying the mRNA levels of the two or more type I interferon (IFN)-inducible genes in a biological sample. In specific aspects, the type I IFNGS is determined by assaying the mRNA levels of the 21 type I interferon (IFN)-inducible genes in a biological sample. In certain embodiments, the biological sample is a test biological sample. In other embodiments, the biological sample is a normal biological sample.

In certain aspects, the type I IFNGS is measured in test biological samples taken from the subject. In other aspects, the pDCs are measured in test biological samples taken from the subject. The biological sample includes, but is not limited to, blood, sputum, saliva, skin cells, skin biopsy samples, kidney cells, lung cells, liver cells, heart cells, brain cells, nervous tissue, thyroid cells, eye cells, skeletal muscle cells, cartilage, bone tissue, cells from airway passages, and cultured cells. In certain embodiments, the biological sample is blood. In other embodiments, the biological sample is tissue. In more specific embodiments, the sample is a tissue comprising skin cells. In other aspects, the sample is a skin biopsy sample.

By “test biological sample” is intended any biological sample obtained from an individual affected, likely to be affected, or suspected to be affected with a disease or condition such as an autoimmune disorder and/or from an individual exhibiting one or more symptoms thereof, such as but not limited to elevated type I IFNGS.

By “normal biological sample” is intended any biological sample obtained from a normal subject.

As used herein, the term “subject” refers to any individual, e.g., a human or a non-human mammal, for whom diagnosis, prognosis, or therapy is desired. The term “subject” may mean a human or non-human mammal affected, likely to be affected, or suspected to be affected with a disease, e.g., an autoimmune disease or condition. The terms “subject” and “patient” are used interchangeably herein. Although the ILT7-binding protein compositions provided herein are principally directed to compositions which are suitable for administration to humans, the skilled artisan will understand that such compositions are generally suitable for administration to subjects of all sorts. In certain aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile; fish, or the like.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, the term “normal subject” refers to any healthy individual, e.g., a human or a non-human mammal, not affected with any disease or suspected of being affected with a disease or condition. The term “normal subject” also refers to an individual e.g., a human or a non-human mammal, prior to exhibiting any symptoms associated with an autoimmune disorder, such as elevated type I IFNGS. The normal subject can be the same subject as the subject in need of treatment, prior to the subject exhibiting any symptoms of an autoimmune disorder, such as but not limited to elevated type I IFNGS. In other embodiments, the normal subject and the subject in need of treatment are two different individuals.

The disclosure provides methods of treating a subject with elevated type I IFNGS comprising administering the ILT7 binding proteins described herein. Patients may exhibit an elevated type I IFNGS when suffering from an autoimmune disorder. Accordingly, the present disclosure provides methods of treating an autoimmune disorder when the subject is exhibiting an elevated type I IFNGS. In some embodiments, the autoimmune disorder is otherwise asymptomatic. In certain aspects, the methods provide selecting a patient for treatment with an ILT7-binding protein, the method comprising: (i) determining the baseline blood type I IFNGS level of the patient, and (ii) selecting those patients with high baseline blood type I IFNGS levels for treatment with the ILT7-binding protein.

As used herein, “treating” or “treat” describes the management and care of a subject for the purpose of combating a disease, condition, or disorder and includes the administration of an ILT7-binding protein used in the methods described herein to alleviate the symptoms or complications of a disease, condition or disorder, or to eliminate the disease, condition or disorder. Thus, the term “treat” or “treating” refers to both therapeutic measures and prophylactic or preventative measures, wherein the objective is to prevent, slow down (lessen), or ameliorate the progression of a disease (e.g., an autoimmune disease). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

In some embodiments, treatment includes the application or administration of the ILT7-binding protein used in the methods described herein to a subject in need thereof or to a subject that is suspected of needing treatment thereof, or application or administration of the ILT7-binding protein used in the methods described herein to an isolated tissue or cell line from a subject, where the subject has a disease, a symptom of a disease, or a predisposition toward a disease (e.g., an autoimmune disease). A subject may be suspected of needing the treatments described herein when the subject is exhibiting symptoms of a condition or disease by excess pDC numbers or activity, even though a formal diagnosis, e.g., the subject has SLE or CLE, has not been ascertained. In certain aspects, the subject suspected of needing treating has a high baseline blood type I IFNGS level. In other embodiments, treatment is also intended to include the application or administration of a pharmaceutical composition comprising a ILT7-binding protein used in the methods described herein to a subject in need thereof or to a subject that is suspected of needing treatment thereof, or application, or administration of a pharmaceutical composition comprising a ILT7-binding protein used in the methods described herein to an isolated tissue or cell line from a subject who has a disease, a symptom of a disease, or a predisposition toward a disease (e.g., an autoimmune disease).

Examples of autoimmune disorders that may be treated when the subject is exhibiting elevated type I IFNGS include but are not limited to systemic lupus erythematosus (SLE), lupus nephritis, cutaneous lupus erythematosus (CLE), Sjögren's syndrome, inflammatory myositis, such as dermatomyositis, inclusion body myositis, juvenile myositis and polymyositis, systemic sclerosis, diabetes, Hashimoto's disease, autoimmune adrenal insufficiency, pure red cell anemia, multiple sclerosis, rheumatic carditis, psoriasis, psoriatic arthritis, rheumatoid arthritis, chronic inflammation, chronic rheumatism, vitiligo, alopecia areata, hidradenitis suppurativa, celiac disease, acute and chronic graft versus host disease (GVHD), vascular inflammation, myocardial infarction, and Type-1 interferonopathies. In certain aspects, the autoimmune disease is SLE. In further aspects, the autoimmune disease is CLE. In certain aspects, the autoimmune disease is lupus, but is not discoid lupus erythematosus (DLE). In other aspects, the autoimmune disease is Sjögren's syndrome. In additional aspects, the autoimmune disease is dermatomyositis. In yet other aspects, the autoimmune disease is polymyositis. In still other aspects, the autoimmune disease is systemic sclerosis. In further other aspects, the autoimmune disease is hidradenitis suppurativa. In still further other aspects, the autoimmune disease is vitiligo.

In still more embodiments, the methods of the present disclosure can be used to monitor the effectiveness of treatment of conditions or disorders by monitoring levels of type I IFNGS and/or activated pDCs. As noted above, autoimmune conditions are often marked by elevated type I IFNGS and/or elevated pDCs, thus monitoring the effectiveness of treatments can include monitoring type I IFNGS and/or pDC levels.

Thus, in certain embodiments, the disclosure provides a method of monitoring effectiveness of treatment of an autoimmune disorder or condition, comprising the steps of: (a) measuring a type I interferon gene signature (IFNGS) in a biological sample taken from the subject to obtain a baseline value of the type I IFNGS; and (b) measuring the type I IFNGS in a biological sample taken from the subject after administering a treatment, wherein the treatment comprises administering an ILT7-binding protein, and wherein a decrease in the type I IFNGS in step (b) compared to the baseline value indicates that the treatment is effective in the subject.

In certain embodiments, the treatment results in a decrease in the type I IFNGS compared to the baseline value. In certain embodiments, the decrease in the type I IFNGS compared to the baseline value ranges from about 1% to about 99%. In certain aspects, the decrease in the type I IFNGS compared to the baseline value is at least about 5%, at least about 10%, at least about 20%, at least about 30%, at least about 40%, at least about 50%, at least about 60%, at least about 70%, at least about 80%, at least about 90%, at least about 95%, or at least about 99%. In some aspects, the decrease in the type I IFNGS compared to the baseline value is at least about 30%. In specific aspects, the decrease in the type I IFNGS compared to the baseline value is at least about 50%.

In certain embodiments, the elevation in type I IFNGS in a test biological sample relative to a normal biological sample, or in a subject in need of treatment with an ILT7 binding protein relative to a normal subject is at least a fold change of about 1.1 to about 1000. Thus, in some embodiments, the type I IFNGS is elevated by at least about 1.1, 1.2, 1.3, 1.4, 1.5, 2, 3, 4, 5, 6, 7, 8, 9, 10, 15, 20, 30, 40, 50, 60, 70, 80, 90, or 100-fold in a test biological sample relative to a normal biological sample, or in a subject in need of treatment with an ILT7 binding protein relative to a normal subject. In specific embodiments, the type I IFNGS is elevated by at least about 4-fold in the test biological sample relative to the normal biological sample, or in a subject in need of treatment with an ILT7 binding protein relative to a normal subject.

Generally, the terms “ILT7-binding protein,” “ILT7-binding molecule,” and “ILT7-binding protein used in the methods described herein” are used interchangeably to refer to a protein or molecule that specifically binds to immunoglobulin-like transcript 7 (ILT7). The terms protein and peptide can be used interchangeably herein. In some embodiments, the ILT7-binding proteins used in the methods described herein bind to full-length ILT7. In other embodiments, the ILT7-binding proteins used in the methods described herein bind to a fragment of ILT7. In certain aspects, the fragment of ILT7 to which the ILT7 binding proteins bind comprises the extracellular domain of ILT7.

In certain embodiments, the ILT7-binding proteins used in the methods disclosed herein bind to any mammalian ILT7. In specific aspects, the ILT7-binding proteins used in the methods disclosed herein bind to human ILT7 or a fragment thereof, for example the extracellular portion of human ILT7. In other aspects, the ILT7-binding proteins used in the methods disclosed herein bind to cynomolgus ILT7 or a fragment thereof, for example the extracellular portion of cynomolgus ILT7.

Examples of ILT7-binding proteins are disclosed and described in PCT Publication No. WO 2017/156298, which is incorporated by reference herein in its entirety. In certain aspects, the ILT7 to which the IL7T binding protein binds is located on pDCs. In specific embodiments, the ILT7-binding protein is VIB7734 antibody or a fragment thereof. VIB7734 is described in PCT Publication No. WO 2017/156298, which is incorporated by reference in its entirety. Specifically, VIB7734 is identified as clone ILT70137 in PCT Publication No. WO 2017/156298. In another embodiment, VIB7734 is also an antibody comprising a heavy chain variable region (VH) of SEQ ID NO:1 and a light chain variable region (VL) of SEQ ID NO:2.

In certain embodiments, the ILT7-binding proteins used in the methods described herein comprise a heavy chain variable region (VH) of SEQ ID NO:1. In other embodiments, the ILT7-binding proteins used in the methods described herein comprise a light chain variable region (VL) of SEQ ID NO:2. In certain aspects, the ILT7-binding proteins used in the methods described herein comprise a heavy chain variable region (VH) of SEQ ID NO:1 and a light chain variable region (VL) of SEQ ID NO:2. In other aspects, the ILT7-binding proteins used in the methods described herein comprise a VH that is at least 85%, 90%, 95%, 96%, 97%, 98% or 99% identical to SEQ ID NO:1 and/or a VL that is at least 85%, 90%, 95%, 96%, 97%, 98% or 99% identical to SEQ ID NO:2.

In more specific embodiments, the ILT7-binding proteins used in the methods described herein comprise heavy chain Complementarity-Determining Regions (HCDRs), HCDR1, HDR2, HCDR3, and light chain Complementarity Determining Regions (LCDRs), LCDR1, LCDR2, and LCDR3 having the amino acid sequences of SEQ ID NOs: 3, 4, 5, 6, 7, and 8, respectively. In other aspects, the ILT7-binding proteins used in the methods described herein comprise heavy chain Complementarity-Determining Regions (HCDRs), HCDR1, HDR2, HCDR3, and light chain Complementarity Determining Regions (LCDRs), LCDR1, LCDR2, and LCDR3, that are at least 85%, 90%, 95%, 96%, 97%, 98% or 99% identical to SEQ ID NOs: 3, 4, 5, 6, 7, and 8, respectively.

In certain embodiments, the ILT7-binding proteins used in the methods described herein may contain fucose moieties or they may be afucosylated.

Without having to be bound by theory, the ILT7-binding proteins used in the methods described herein induce antibody-dependent cell-mediated cytotoxicity (ADCC) activity against plasmacytoid dendritic cells (pDCs), thereby depleting pDCs. In certain aspects, ILT7-binding protein-mediated ADCC causes a reduction in circulating pDCs. In certain aspects, ILT7-binding protein-mediated ADCC causes a reduction in local or tissue pDCs. In certain embodiments, the tissue in which the pDCS are reduced includes, but is not limited to, skin cells, skin biopsy samples, kidney cells, lung cells, liver cells, heart cells, brain cells, nervous tissue, thyroid cells, eye cells, skeletal muscle cells, cartilage, bone tissue, and cells from airway passages. In some aspects, the tissue is a skin biopsy sample. In more specific aspects, administering the ILT7-binding proteins will cause a reduction in skin pDCs.

Normally, pDCs are not present in skin tissue, and immature pDCs are typically only found in blood, thymus lymphoid tissue, tonsils and lung tissue. Thus the presence of pDCs in skin biopsy samples is indicative of an abnormal condition in which pDCs are recruited to the skin. Accordingly, the methods of the present disclosure include administering an ILT7-binding protein to a subject in need of treatment of a condition marked by the presence of pDCs in the subject's skin. The methods of the present disclosure include reducing the levels of pDCs in a subject's skin by administering an ILT7-binding protein to the subject in need of treatment thereof.

In some embodiments, subjects have an elevated or high level of pDCs in skin tissue prior to treatment. In certain embodiments, subjects with a high pDC level in skin tissue prior to treatment are more responsive to the treatment. In certain aspects, the subjects with a high pDC level in skin tissue have a pDC level of at least about 50 pDC/mm² of skin tissue, at least about 60 pDC/mm² of skin tissue, at least about 70 pDC/mm² of skin tissue, at least about 80 pDC/mm² of skin tissue, at least about 90 pDC/mm² of skin tissue, at least about 100 pDC/mm² of skin tissue, at least about 110 pDC/mm² of skin tissue, at least about 120 pDC/mm² of skin tissue, at least about 125 pDC/mm² of skin tissue, at least about 150 pDC/mm² of skin tissue, at least about 175 pDC/mm² of skin tissue, at least about 200 pDC/mm² of skin tissue, or higher. In certain embodiments, a low pDC level in skin tissue is considered less than about 10 pDC/mm² of skin tissue. In specific embodiments a high pDC level in skin tissue is considered at least about 100 pDC/mm² of skin tissue.

In other embodiments, the methods of the present disclosure comprise administering an ILT7-binding proteins used in the methods described herein to suppress release of type I IFN from pDCs, regardless of the location of the pDCs. In other embodiments, the methods of the present disclosure comprise administering an ILT7-binding protein to suppress release of type I IFN from pDCs in the blood or circulation. In other embodiments, the methods of the present disclosure comprise administering an ILT7-binding protein to suppress release of type I IFN from local pDCs. In other embodiments, the methods of the present disclosure comprise administering an ILT7-binding protein to suppress release of type I IFN from pDCs in the skin of the subject. In certain embodiments, the type I IFN that suppressed in its release is IFNα. In certain aspects, ILT7-binding protein-mediated suppression of release of type I IFN from pDCs causes a reduction in type I IFNGS.

The term “reduce,” “reducing,” or “reduction” means to diminish in extent, level, amount, activity, or degree compared to an initial value. The reduction need not be statistically significant from one value over the next.

The terms “administer,” “administration,” “administering” and the like, as they apply to, for example, a subject, cell, tissue, organ, or biological sample, refer to contact of a compound or reagent to the subject, cell, tissue, organ, or biological sample. In the context of a cell, administration includes contact (e.g., in vitro or ex vivo) of a reagent to the cell, as well as contact of a reagent to a fluid, where the fluid is in contact with the cell. The ILT7-binding proteins used in the method described herein may be administered to a subject via a variety of routes known in the art. Exemplary routes of administering of the ILT7-binding proteins used in the methods described herein include, but are not limited to, parenteral, oral, mucosal, topical, transdermal, inhalation, sublingual, buccal, rectal, vaginal, and intranasal. The term parenteral, as used herein, includes subcutaneous injections, intravenous, intramuscular, intrasternal injection or infusion techniques. In certain aspects, the ILT7-binding proteins used in the methods described herein are administered intravenously. In specific aspects, the ILT7-binding proteins used in the methods described herein are administered by subcutaneous injection. The term “administer,” “administration,” or “administering” may involve a single administration or multiple administrations of an ILT7-binding protein used in the methods described herein. For example, multiple administration involves at least two (i.e., 2, 3, 4, 5, 6, 7, 8, 9, 10, or more) administrations to a subject of an ILT7-binding protein used in the methods described herein.

A “therapeutically effective amount,” or “pharmaceutically effective amount,” or “effective amount” of a compound (e.g., an ILT7-binding protein used in the methods described herein) refers to an amount that is sufficient to produce a desired prophylactic, therapeutic or ameliorative response in a subject, or an amount that is sufficient to result in prevention or amelioration of one or more symptoms of a disease or condition in a statistically significant manner. When referring to an individual active ingredient administered alone, a therapeutically effective dose refers to that ingredient alone. When referring to a combination, a therapeutically effective dose refers to combined amounts of the active ingredients that result in the therapeutic effect, whether administered serially or simultaneously. As used herein, the term “therapeutically effective amount” means that the ILT7-binding proteins used in the methods described herein are able to exert a medically beneficial effect (e.g., cause a reduction in an elevated type I IFNGS and/or reduction in pDCs in a subject in need thereof) when used as prescribed or directed, as compared to a placebo. The therapeutically effective amount will vary depending upon the species and weight of the subject to be administered, but may be ascertained using standard techniques. In certain embodiments, a therapeutically effective amount of an ILT7-binding protein used in the methods described herein ranges from about 0.1 mg to about 1000 mg. In other embodiments, a therapeutically effective amount of an ILT7-binding protein used in the methods described herein ranges from about 50 mg to about 150 mg. In certain aspects, a therapeutically effective amount of an ILT7-binding protein used in the methods described herein includes, but is not limited to, about 1 mg, about 5 mg, about 15 mg, about 50 mg, about 100 mg, about 150 mg, about 300 mg, about 500 mg, or about 1000 mg. In certain embodiments, a therapeutically effective amount of an ILT7-binding protein used in the methods described herein is about 5 mg in a single dose. In other embodiments, a therapeutically effective amount of an ILT7-binding protein used in the methods described herein is about 50 mg in a single dose. In yet other embodiments, a therapeutically effective amount of an ILT7-binding protein used in the methods described herein is about 150 mg in a single dose. A therapeutically effective amount of an ILT7-binding protein used in the methods described herein may be administered to a subject in need thereof in a single dose or in multiple doses.

In certain embodiments, administration of a therapeutically effective amount of an ILT7-binding protein used in the methods described herein to a subject in need thereof leads to about 1% to about 100% reduction in type I IFNGS in the subject compared to the type I IFNGS prior to administration of the ILT7-binding protein used in the methods described herein. In certain aspects, administration of a therapeutically effective amount of an ILT7-binding protein used in the methods described herein to a subject in need thereof leads to at least about 1%, at least about 2%, at least about 5%, at least about 10%, at least about 20%, at least about 30%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% reduction in type I IFNGS in the subject, compared to the type I IFNGS prior to administration of the ILT7-binding protein used in the methods described herein. In specific embodiments, administration of a therapeutically effective amount of the ILT7-binding protein leads to at least about 50% reduction in the type I IFNGS in the subject.

In certain embodiments, administration of the ILT7-binding protein used in the methods described herein to a subject in need thereof leads to at least about 50% reduction in the type I IFNGS in the subject, compared to the type I IFNGS prior to administration of the ILT7-binding protein used in the methods described herein. In certain aspects, administration of a therapeutically effective amount of an ILT7-binding protein used in the methods described herein to a subject in need thereof leads to the at least about 50% reduction in type I IFNGS in the subject at about 8 hours, about 12 hours, about 24 hours, or about 48 hours following administration of the ILT7-binding protein.

In specific embodiments, a subject who has been administered a therapeutically effective amount of an ILT7-binding protein used in the methods described herein shows a reduction in type I IFNGS of at least about 50% at about 24 hours following administration of the ILT7-binding protein, compared to the type I IFNGS in the subject prior to administration of the ILT7-binding protein.

In certain embodiments, the reduction in type I IFNGS persists for at least about 1 day, at least about 2 days, at least about 3 days, at least about 4 days, at least about 5 days, at least about 6 days, at least about 7 days, at least about 14 days, at least about 21 days, at least about 28 days, at least about 30 days, at least about 45 days, at least about 60 days, at least about 90 days, or at least about 180 days or longer following administration of a therapeutically effective amount of an ILT7-binding protein used in the methods described herein to a subject in need thereof. In some aspects, the reduction in type I IFNGS persists for up to about 30 days following administration of a therapeutically effective amount of an ILT7-binding protein used in the methods described herein to a subject in need thereof. In additional aspects, the reduction in type I IFNGS persists for up to about 60 days following administration of a therapeutically effective amount of an ILT7-binding protein used in the methods described herein to a subject in need thereof. Therefore, in some embodiments, a therapeutically effective amount of an ILT7-binding protein used in the methods described herein is administered to a subject in need thereof at least once every month. In other embodiments, a therapeutically effective amount of an ILT7-binding protein used in the methods described herein is administered to the subject at least once about every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32,33, 34,3 5, 36, 37, 38 39, 40, 41, 42,43, 44, 45, 46, 47, 48, 49, 50, 51, or 52 weeks. In some embodiments, a therapeutically effective amount of an ILT7-binding protein used in the methods described herein is administered to the subject at least once every 4 weeks. In additional embodiments, a therapeutically effective amount of an ILT7-binding protein used in the methods described herein is administered to the subject at least once every 8 weeks or at least once every 12 weeks. In further embodiments, a therapeutically effective amount of an ILT7-binding protein used in the methods described herein is administered to the subject at least once every two or three months. In still further embodiments, a therapeutically effective amount of an ILT7-binding protein used in the methods described herein is administered to the subject at least once every year or at least once every 2 years.

As used herein, the term “reduction in pDCs” or “reducing pDCs” refers to diminished levels of activated pDCs in a subject or in a biological sample (e.g., blood and/or other tissues such as skin cells, skin biopsy samples, etc.) taken from the subject, diminished levels of the total number of pDCs in a subject or in a biological sample taken from the subject, or both. In some embodiments, the reduction in pDCs in the subject is about 1% to about 100% compared to the pDCs in the subject prior to administration of an ILT7-binding protein used in the methods described herein. In certain aspects, the a reduction in pDCs in the subject is at least about 1%, at least about 2%, at least about 5%, at least about 10%, at least about 20%, at least about 30%, at least about 30%, at least about 40%, at least about 50%, at least about 60%, at least about 70%, at least about 80%, at least about 90%, at least about 95%, at least about 96%, at least about 97%, at least about 98%, at least about 99%, or about 100% compared to pDCs in the subject prior to administration of an ILT7-binding protein used in the methods described herein. In specific embodiments, the reduction in pDCs in the subject is at least about 50% compared to pDCs in the subject prior to administration of an ILT7-binding protein used in the methods described herein. Thus, in certain embodiments, administration of a therapeutically effective amount of the ILT7-binding protein leads to at least about 10% reduction in total number of pDCs in the subject. In additional embodiments, administration of a therapeutically effective amount of the ILT7-binding protein leads to at least about 10% reduction in activated pDCs in the subject. In certain aspects, the pDCs are measured in a test biological sample taken from the subject. Therefore, in certain embodiments, administering a therapeutically effective amount of an ILT7-binding protein used in the methods described herein to a subject in need thereof leads to a reduction in pDCs in a test biological sample taken from the subject. In specific embodiments, the reduction in pDCs in a test biological sample taken from the subject is at least about 10% compared to pDCs in the test biological sample prior to administration of an ILT7-binding protein used in the methods described herein. In certain aspects, the test biological sample is blood. In specific aspects, the test biological sample is tissue, including, but not limited to, skin cells and skin biopsy specimens. In certain aspects, the pDCs are circulating pDCs. In other aspects, the pDCs are pDCs in the skin. In additional aspects, the reduction in pDCs is reversible.

In certain embodiments, administration of a therapeutically effective amount of an ILT7-binding protein used in the methods described herein to a subject in need thereof causes at least about 10% reduction in pDCs in the subject at about 5 minutes, at about 10 minutes, about 15 minutes, about 30 minutes, about 1 hour, about 2 hours, about 3 hours, about 6 hours, about 12 hours, about 24 hours, or about 48 hours following administration of the ILT7-binding protein. In other embodiments, administration of a therapeutically effective amount of an ILT7-binding protein used in the methods described herein to a subject in need thereof causes at least about 10% reduction in pDCs in a test biological sample taken from the subject at about 5 minutes, at about 10 minutes, about 15 minutes, about 30 minutes, about 1 hour, about 2 hours, about 3 hours, about 6 hours, about 12 hours, about 24 hours, or about 48 hours following administration of the ILT7-binding protein.

In certain embodiments, the reduction in pDCs persists for at least about 1 day, at least about 2 days, at least about 3 days, at least about 4 days, at least about 5 days, at least about 6 days, at least about 7 days, at least about 14 days, at least about 21 days, at least about 28 days, at least about 30 days, at least about 45 days, at least about 60 days, at least about 90 days, or at least about 180 days or longer following administration of a therapeutically effective amount of an ILT7-binding protein used in the methods described herein to a subject in need thereof. In some aspects, the reduction in pDCs persists for at least about 30 days following administration of a therapeutically effective amount of an ILT7-binding protein used in the methods described herein to a subject in need thereof. In additional aspects, the reduction in pDCs persists for at least about 60 days following administration of a therapeutically effective amount of an ILT7-binding protein used in the methods described herein to a subject in need thereof.

In other embodiments, the methods of the present disclosure can be used for reducing Cutaneous Lupus Erythematosus Disease Activity and Severity Index (CLASI) in a tissue of a subject in need thereof. The methods comprise administering to the subject a pharmaceutically effective amount of an ILT7-binding protein.

As used herein, the term “CLASI” refers to Cutaneous Lupus Erythematosus Disease Activity and Severity Index. The CLASI is a validated instrument for measuring skin manifestations of CLE. The CLASI consists of two scores: the first summarizes the inflammatory activity of the disease; the second is a measure of the damage done by the disease. The activity score includes erythema (0-3), scale/hypertrophy (0-2), mucous membrane lesions (0-1), recent hair loss (0-1) and non-scarring alopecia (0-3). The damage score represents dyspigmentation (0-1), scarring/atrophy/panniculitis (0-2), and scarring of the scalp (0-6). Patients are asked if their dyspigmentation lasts 12 months or longer, in which case, the dyspigmentation score is doubled. Each of the above parameters is measured in 13 different anatomical locations, included specifically because they are most often involved in CLE. The most severe lesion in each area is measured.

As used herein, the term “reduction CLASI” refers diminished levels of CLASI-Activity (CLASI-A) score in a subject or in a biological sample (e.g., issues such as skin cells, skin biopsy samples, etc.) taken from the subject, or diminished levels of CLASI-Damage (CLASI-D) score in a subject or in a biological sample taken from the subject, or both.

Thus, in certain aspects, the methods of the disclosure result in a reduced CLASI-A score in the subject. In certain aspects, a reduction in the CLASI-A score of a subject involves a reduction of the CLASI-A score by at least 1, 2, 3, 4, 5, 6, 7, 8, 9, or 10 points from a baseline value. In some embodiments, a reduction in the CLASI-A score of a subject involves a reduction of the CLASI-A score by at least 4 points from a baseline value. In specific embodiments, a reduction in the CLASI-A score of a subject involves a reduction of the CLASI-A score by at least 7 points from a baseline value. In other embodiments, a reduction in the CLASI-A score of a subject involves a reduction of the CLASI-A score by at least 5%, 10%, 15%, 20%, 25%, 30%, 35%, 40%, 45%, 50%, 55%, 60%, 65%, 70%, 75%, 80%, 85%, 90%, 95%, or 99% from a baseline value. In specific embodiments, a reduction in the CLASI-A score of a subject involves a reduction of the CLASI-A score by at least 50% from a baseline value. In certain aspects, the baseline value is the value of the CLASI-A score in the subject prior to treatment with an ILT7-binding protein used in the methods described herein. In other aspects, the methods of the present disclosure result in a reduced CLASI-D score in the subject. In additional aspects, the methods of the present disclosure result in a reduced CLASI-A score and a reduced CLASI-D score in the subject.

### Pharmaceutical Compositions

The present disclosure is also directed to pharmaceutical compositions comprising the ILT7-binding proteins used in the methods described herein. In certain embodiments, the present disclosure provides for the use of an ILT7-binding protein used in the methods described herein in the manufacture of a medicament for treating a subject.

In some embodiments, a pharmaceutical composition of the disclosure comprises an ILT7-binding protein disclosed herein and one or more pharmaceutically acceptable carriers, diluents, or excipients. In this regard, “pharmaceutically acceptable carriers, diluents, or excipients” include but are not limited to any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that may or may not have been approved by the United States Food and Drug Administration as being acceptable for use in humans or domestic animals. For example, appropriate carriers are known to those skilled in the art and include stabilizers, diluents, and buffers. Suitable stabilizers include carbohydrates, such as sorbitol, lactose, mannitol, starch, sucrose, dextran, and glucose, and proteins, such as albumin or casein. Suitable diluents include saline, Hanks Balanced Salts, and Ringers solution. Suitable buffers include an alkali metal phosphate, an alkali metal carbonate, or an alkaline earth metal carbonate.

In certain aspects, the pharmaceutical compositions of the disclosure may further contain one or more auxiliary substance, such one or more lipids, phospholipids, carbohydrates, and lipopolysaccharides. In some embodiments, pharmaceutical compositions of the disclosure optionally comprise one or more additional active substances.

In certain cases, the pharmaceutical compositions of the disclosure can be prepared by techniques known to those skilled in the art. General considerations in the formulation and/or manufacture of pharmaceutical compositions may be found, for example, in Remington: The Science and Practice of Pharmacy 21st ed., Lippincott Williams & Wilkins, 2005 (incorporated herein by reference in its entirety). Generally, an ILT7-binding protein used in the methods described herein or fragments thereof is mixed with a carrier to form a solution, suspension, or emulsion. One or more of the additives discussed herein may be added in the carrier or may be added subsequently. The pharmaceutical compositions of the disclosure may be an aqueous solution, emulsion or suspension or may be a dried preparation. In certain aspects, the pharmaceutical compositions of the disclosure may be desiccated or lyophilized, for example, by freeze drying or spray drying for storage or formulations purposes. They may be subsequently reconstituted into liquid compositions by the addition of an appropriate liquid carrier or administered in dry formulation using methods known to those skilled in the art. In certain embodiments, the ILT7-binding proteins used in the methods described herein are stored as lyophilized powder and subsequently reconstituted into liquid compositions prior to administration into a subject in need thereof.

The choice of administration of the pharmaceutical composition will depend on the formulation that is selected. The pharmaceutical compositions of the disclosure are administered in a manner compatible with the dosage formulation, and in such amount as will be therapeutically effective. In certain aspects, a pharmaceutical composition of the disclosure is formulated into preparations in solid, semi-solid, liquid or gaseous forms, including, but not limited to, tablets, capsules, powders, granules, ointments, solutions, suppositories, injections, inhalants, gels, microspheres, and aerosols.

In certain instances, a pharmaceutical composition comprising an ILT7-binding protein used in the methods described herein may be in the form of a solid or liquid. In some aspects, the carrier(s) are particulate so that the compositions are, for example, in tablet or powder form. In other aspects, the carrier(s) are liquid, with a composition being, for example, an oral syrup, injectable liquid or an aerosol, which is useful in, for example, inhalatory administration. When intended for oral administration, a pharmaceutical composition comprising an ILT7-binding protein used in the methods described herein is in either solid or liquid form, where semi-solid, semi-liquid, suspension and gel forms are included within the forms considered herein as either solid or liquid.

In certain aspects, as a solid composition for oral administration, a pharmaceutical composition comprising an ILT7-binding protein used in the methods described herein may be formulated into a powder, granule, compressed tablet, pill, capsule, chewing gum, wafer or the like form. In some instances, such a solid composition will typically contain one or more inert diluents or edible carriers. In certain embodiments, one or more of the following may be additionally present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

In some aspects, when a pharmaceutical composition of the disclosure is in the form of a capsule, for example, a gelatin capsule, it may contain, in addition to materials disclosed herein, a liquid carrier such as polyethylene glycol or oil. Oral formulations may also include normally employed incipients such as, for example, pharmaceutical grades of saccharine, cellulose and magnesium carbonate.

In other aspects, a pharmaceutical composition of the disclosure is in the form of a liquid, for example, an elixir, syrup, solution, emulsion or suspension. In certain embodiments, the liquid may be for oral administration or for delivery by injection. In certain embodiments, when intended for oral administration, the pharmaceutical compositions of the disclosure contain, in addition to an ILT7-binding protein used in the methods described herein, one or more of a sweetening agent, preservatives, dye/colorant and flavor enhancer. In certain aspects, in a pharmaceutical composition intended to be administered by injection, one or more of a surfactant, preservative, wetting agent, dispersing agent, suspending agent, buffer, stabilizer and isotonic agent may be included. In certain aspects, a pharmaceutical composition of the disclosure is administered to a subject in need thereof intravenously. In specific aspects, a pharmaceutical composition of the disclosure is administered to a subject in need thereof by subcutaneous injection.

In certain cases, liquid pharmaceutical compositions comprising an ILT7-binding protein used in the methods described herein, whether they be solutions, suspensions or other like form, may include one or more of the following components: sterile diluents such as water for injection, saline solution, e.g., physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In some cases, the preparation can be enclosed in ampoules, disposable syringes or multiple dose vials made of glass or plastic. In some embodiments, an injectable pharmaceutical composition is preferably sterile.

In other embodiments, a pharmaceutical composition comprising an ILT7-binding protein used in the methods described herein may be intended for topical administration, in which case the carrier may suitably comprise a solution, emulsion, ointment or gel base. In certain aspects, the base, for example, may comprise one or more of the following: petrolatum, lanolin, polyethylene glycols, bee wax, mineral oil, diluents such as water and alcohol, and emulsifiers and stabilizers. In other aspects, thickening agents may be present in a pharmaceutical composition for topical administration. In certain embodiments, if intended for transdermal administration, a pharmaceutical composition of an ILT7-binding protein used in the methods described herein may be included with a transdermal patch or iontophoresis device.

In yet other embodiments, the pharmaceutical composition comprising an ILT7-binding protein used in the methods described herein is intended for rectal administration, in the form, for example, of a suppository. For suppositories, binders and carriers may include, for example, polyalkalene glycols or triglycerides. In certain instances, a composition for rectal administration contains an oleaginous base as a suitable nonirritating excipient. Such bases include, without limitation, lanolin, cocoa butter or polyethylene glycol.

In other aspects, a pharmaceutical composition comprising an ILT7-binding protein used in the methods described herein comprises dosage units that can be administered as an aerosol. The term aerosol is used to denote a variety of systems ranging from those of colloidal nature to systems consisting of pressurized packages. In certain embodiments, delivery is accomplished by a liquefied or compressed gas or by a suitable pump system that dispenses the active ingredients. In some embodiments, aerosols of an ILT7-binding protein used in the methods described herein may be delivered in single phase, bi-phasic, or tri-phasic systems in order to deliver the active ingredient(s). In other embodiments, delivery of the aerosol includes the necessary container, activators, valves, subcontainers, and the like, which together may form a kit. One skilled in the art can readily determine specific aerosol formulations and delivery modes.

Pharmaceutical compositions of the disclosure may be administered in a suitable, nontoxic pharmaceutical carrier, may be comprised in microcapsules, microbeads, and/or may be comprised in a sustained release implant.

In some aspects, pharmaceutical compositions of the disclosure include materials that form a coating shell around the active ingredients. In some instances, the materials that form the coating shell are typically inert, and may be selected from, for example, sugar, shellac, and other enteric coating agents.

In yet other aspects, the pharmaceutical compositions of the disclosure in solid or liquid form include an agent that binds to an ILT7-binding protein used in the methods described herein and thereby assist in the delivery of the ILT7-binding protein used in the methods described herein. In certain cases, suitable agents that act in this capacity include a protein or a liposome.

In certain aspects, pharmaceutical compositions that will be administered to a subject take the form of one or more dosage units, where, for example, a tablet may be a single dosage unit, and a container of an ILT7-binding protein used in the methods described herein in aerosol form may hold a plurality of dosage units. Actual methods of preparing such dosage forms are known, or will be apparent to those skilled in this art; for example, see Remington: The Science and Practice of Pharmacy, 20th Edition (Philadelphia College of Pharmacy and Science, 2000). A composition to be administered will, in any event, contain a therapeutically effective amount of an ILT7-binding protein used in the methods described herein, or a pharmaceutically acceptable salt thereof, to aid in treatment of a disease or condition of interest in accordance with the teachings herein.

In certain embodiments, the pharmaceutical compositions of the disclosure comprise one or more additional therapeutically active substances. In other embodiments, a therapeutically effective dose of the pharmaceutical compositions of the disclosure is administered to a subject in need thereof in combination with one or more additional therapeutically active substances. As used herein, a “combination” refers to a combination comprising an ILT7-binding protein used in the methods described herein and one or more additional therapeutically active substances, each of which may be administered serially (sequentially), concurrently or simultaneously.

Pharmaceutical compositions of the disclosure may desirably be administered at several intervals in order to sustain therapeutic levels. Pharmaceutical compositions of the disclosure may be used in conjunction with other bacteriocidal or bacteriostatic methods.

Although the descriptions of pharmaceutical compositions provided herein are principally directed to pharmaceutical compositions which are suitable for administration to humans, it will be understood by the skilled artisan that such compositions are generally suitable for administration to subjects of all sorts. In certain aspects, the subject is a mammal. In certain aspects, a mammal includes primates, such as humans, monkeys and apes, and non-primates such as domestic animals, including laboratory animals and household pets and farm animals (e.g., cats, dogs, swine, cattle, sheep, goats, horses, rabbits), and non-domestic animals, such as wildlife, birds, or the like.

### EXAMPLES

In autoimmune diseases, upon activation by immune complexes to self-nucleic acids, plasmacytoid dendritic cells (pDCs) secrete significant amounts of type I and type III interferons (IFNs). pDCs constitute about 0.4% of circulating white blood cells and can recognize viral nucleic acids, which are often bound to other proteins or to immunoglobulins. Although this response is believed to contribute to antiviral defense, evidence has accumulated that pDCs and type I IFNs also contribute to the pathogenesis of numerous autoimmune diseases. Type I IFN levels cannot be directly measured in a reliable way; however, binding of type I IFN to its receptor leads to local and systemic upregulation of type I IFN-inducible genes. The messenger ribonucleic acid (mRNA) levels of these type I IFN-inducible genes can be measured in blood and analyzed as a composite outcome referred to as the “type I interferon gene signature” (IFNGS). A test for the type I IFNGS was developed and cut-off values were established to score these signatures as “IFN test-high” and “IFN test-low”. Studies using this specific gene signature found that a subset of patients with an elevated type I IFN signature was identifiable in systemic lupus erythromatosus, dermatomyositis, polymyositis, systemic sclerosis, and Sjögren's syndrome.

### Single Ascending Dose (SAD) Study

A Phase 1a, randomized, site-blinded/sponsor-unblinded, placebo-controlled trial of a single escalating subcutaneous dose of the ILT7-binding protein used in the methods described herein was carried out in 5 successive cohorts of patients with systemic lupus erythematosus (SLE), Sjögren's syndrome (SS), dermatomyositis (DM), polymyositis (PM), or systemic sclerosis (SSc). The trial evaluated the safety, drug levels, pDC levels, anti-drug antibodies (ADA), and impact on a 21-gene type I IFNGS of the ILT7-binding protein used in the methods described herein (VIB7734).

All data were presented in the form of listings sorted by cohort, treatment, and subject number. Tabular summaries of the data collected were presented by treatment group. Categorical data was summarized by the frequency counts and percentage of subjects in each category. Percentages were calculated based on non-missing observations where applicable. Continuous variables were summarized by descriptive statistics including number of observations, mean, standard deviation, median, minimum, and maximum. In general, unless stated otherwise, “baseline” was defined as the last value prior to first dose of VIB7734.

### Example 1: SAD Study Design

A total of 36 subjects were enrolled in the study. The enrolled subjects had the following diagnoses: SLE 19 (53%), SS 16 (44%), SSc 3 (8%), PM 2 (6%), and DM 2 (6%). Baseline demographic characteristics were well-balanced and generally similar between the total VIB7734 group and the placebo group. The majority of the subjects were White and were females (32 [88.9%]). The enrolled subjects were randomized in a 3:1 ratio within 5 cohorts to receive a single subcutaneous dose of VIB7734 or matching placebo as follows:
Cohort 1: 1 mg VIB7734 (n=3) or placebo (n=1);
Cohort 2: 5 mg VIB7734 (n=6) or placebo (n=2);
Cohort 3: 15 mg VIB7734 (n=6) or placebo (n=2);
Cohort 4: 50 mg VIB7734 (n=6) or placebo (n=2);
Cohort 5: 150 mg VIB7734 (n=6) or placebo (n=2).

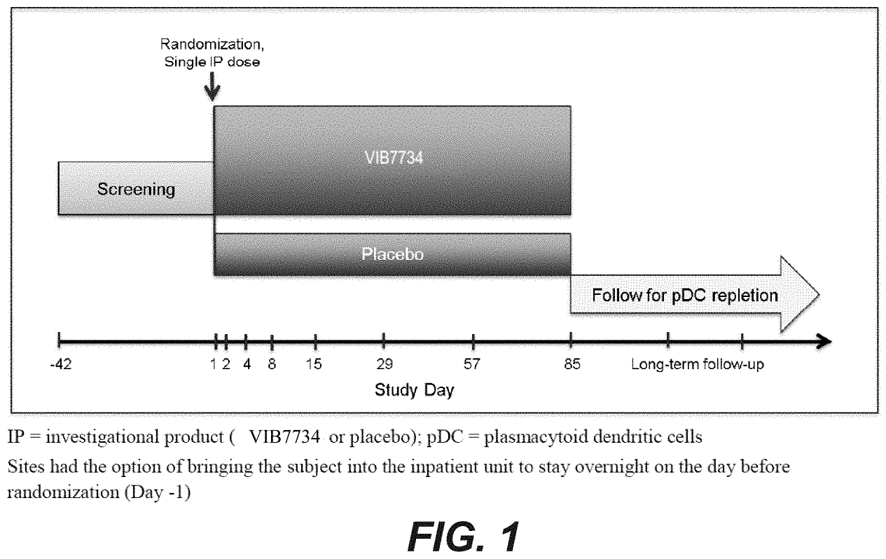

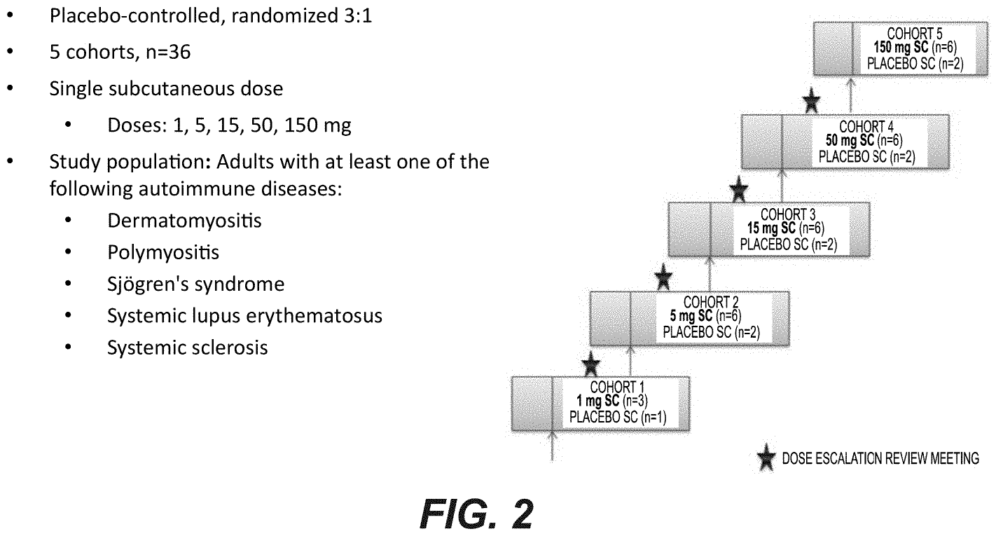

A diagrammatic presentation of the study is provided in FIGS. 1 and 2. A screening visit was performed within 42 days prior to dosing. Subjects received VIB7734 on Day 1 and were observed overnight (in the facility) for any safety concerns. Subjects were discharged on Day 2 after completion of all study procedures and ensuring that there were no safety issues. This was followed by a treatment follow-up period of 85 days. During this period, subjects returned to the study site on Days 4, 8, 15, 29, 57, and 85. The subjects were evaluated for pDC repletion. The pDC repletion criteria was met if pDC level was ≥50% of the subject's baseline value or pDC level was greater than the lower limit of the standard range as defined in the laboratory manual. If they did not meet the pDC repletion criteria at Day 85, they were to be followed up for additional 252 days (to study Day 337). If the pDC repletion criteria was not met by the Day 337 visit, the medical monitor in consultation with the investigator determined the need for further follow-up.

Subjects received only one dose of VIB7734 during the study. As shown in FIG. 2, cohorts were enrolled in a dose-escalating manner to provide time for review of safety and tolerability data before progressing to the next dose level (cohort). The decision to escalate to the next dosing cohort was made by the Dose Escalation Committee (DEC).

In each cohort, there was at least a 48 hour interval between the dosing of first and second subjects and between the second and the third subject. Starting with the third subject in each cohort, there was at least a 24 hour interval between the dosing of subsequent subjects. Dosing for Cohorts 2, 3, 4, and 5 commenced once all subjects in the previous cohort had been randomized and administered with VIB7734, all evaluable subjects had completed at least the Day 15 visit (Visit 6), and the cumulative safety data of all exposed subjects was reviewed by the DEC, who agreed the safety profile to be acceptable.

### Example 2: SAD Study: Evaluation of Adverse Reactions

Serious adverse events occurred in 1 subject in the VIB7734 15 mg group (colitis) and 1 subject in the placebo group (death due to cerebral hemorrhage). At least one adverse event (AE) was reported in 69% of VIB7734-treated, and 80% of placebo-treated subjects. The most commonly reported AEs in VIB7734-treated subjects were diarrhea (12%) and upper respiratory tract infection (12%). No injection site reactions or hypersensitivity reactions occurred.

### Example 3: SAD Study: Immunogenicity Evaluations

Blood samples were collected on Days 1, 2, 4, 8, 15, 29, 57, and 85 to evaluate anti-drug antibody (ADA) response to VIB7734 in human serum. These evaluations were performed utilizing a validated electrochemiluminescence immunoassay method for the detection, and confirmation and titration of anti-drug antibodies to VIB7734 in human serum. Samples found to be negative in the screening tier were reported to have a titer of <30.

Baseline and post-baseline ADA results were recorded for all 26 subjects in total ILT7-binding protein group and 9 subjects in placebo group. No positive results were observed in either treatment groups. No incidence of ADA persistent positive or transient positive was observed in either treatment groups. Thus, overall, for subcutaneous injection of VIB7734 in doses ranging from 1 to 150 mg, no safety, tolerability, or immunogenicity issues were identified.

### Example 4: SAD Study: Pharmacokinetic Evaluations

Blood samples were collected on Days 1, 2, 4, 8, 15, 29, 57, and 85 to evaluate PK of VIB7734 in serum. Concentrations of VIB7734 were measured in human serum samples by utilizing a validated enzyme-linked immunosorbent assay (ELISA) immunoassay method. The validated measurement range of the assay was 0.025 μg/mL to 25.60 μg/mL. Results below the lower limit of quantitation (LLOQ) were reported as <0.10 μg/mL.

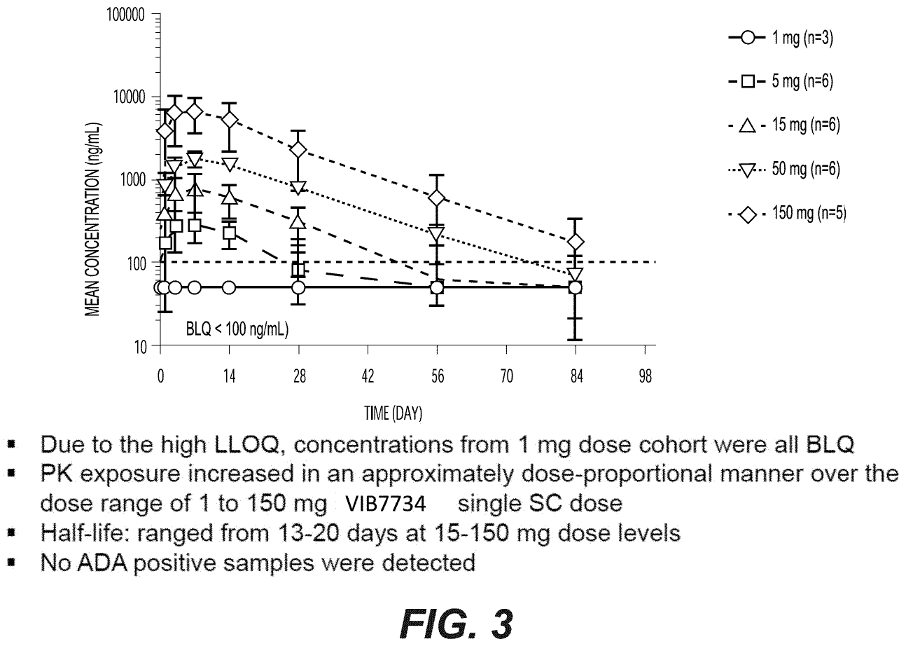

The PK analysis was performed on time data of concentration of VIB7734 from all 26 subjects who received any dose of VIB7734. Mean serum concentration-time profiles of VIB7734 following a single subcutaneous dose of 1, 5, 15, 50, or 150 mg are shown in FIG. 3. Following the 1 mg dose, all concentrations were below the quantitation limit (BLQ), therefore all the summary PK parameters are based on 5-150 mg dose levels. PK exposures of VIB7734 increased approximately dose proportionally. After a single subcutaneous injection on Day 1, peak concentrations were observed in 5 to 8 days post dose. Exposures increased in an approximately dose-proportional manner with increasing dose levels. The estimated half-life ranged from 13 to 20 days across dose levels. Mean extravascular clearance ranged from 468 to 1030 mL/day. Mean extravascular volume of distribution ranged from 9.9 to 19.0 L.

### Example 5: SAD Study: Pharmacodynamic Evaluations (Blood pDC Levels)

Whole blood samples for pDC levels were collected on Days 1, 2, 4, 8, 15, 29, 57, and 85. The baseline pDC level was defined as the mean of the levels measured at Visits 1 and 2. If the screening (Visit 1) sample was not drawn or failed for technical reasons, it was to be repeated and results had to be available before the subject could be randomized since the result was needed to determine that the subject met all inclusion/exclusion criteria. If the Day 1 (Visit 2) sample was not drawn or failed for technical reasons, the value from the Visit 1 sample was to be considered the baseline. The study site was blinded to post-baseline pDC levels.

The pDC levels were quantified in two ways during the study: 1) as a percentage of the CD45+ peripheral blood mononuclear cells (PBMCs, primary method), and 2) as a concentration of pDCs per μL (secondary method). The primary pDC measure is the pDCs as a % of CD45+ PBMCs since that is what is directly measured by the flow cytometry assay used in this study.

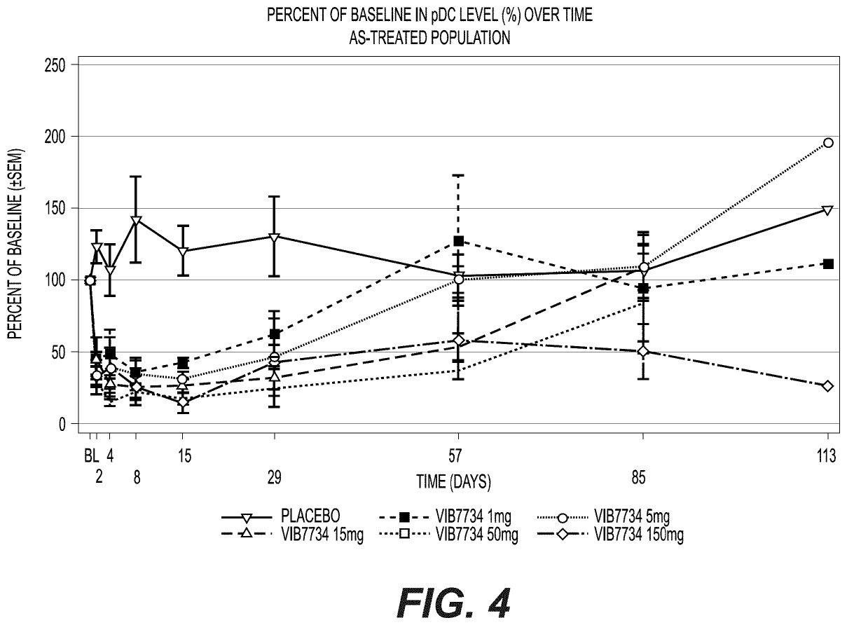

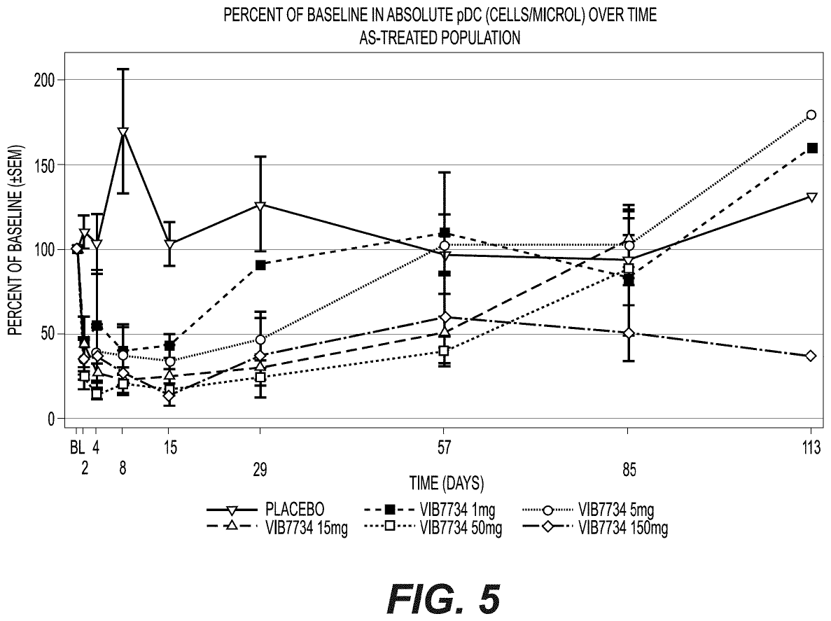

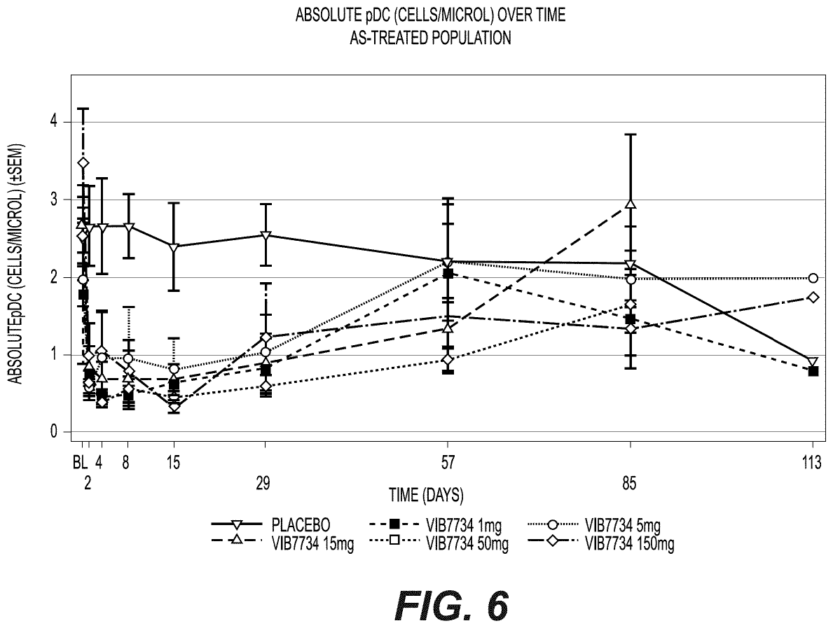

At baseline the mean pDC level in the blood was 0.13% (SD: 0.056%) of PBMCs in the VIB7734-treated subjects. The mean concentration of pDCs at baseline in the VIB7734-treated subjects was 2.53 cells/μL (SD: 1.24%). The levels and change from baseline in pDC (% of CD45+ cells) over time is presented in FIG. 4. The levels and change from baseline in absolute concentration of pDC over time is presented in FIG. 5. The changes in absolute pDC levels over time is presented in FIG. 6.

There was decrease in the blood level of pDCs after SC administration of all tested doses of VIB7734. Median reductions of at least 50% in the pDC level of VIB7734-treated subjects were evident at 24 hours after dosing (the first blood draw done after dosing) in all VIB7734 dose groups, with a maximal reduction of 90%. Increasing doses were associated with a non-linear increase in pDC reduction. At Day 15, median pDC levels changed as follows for the VIB7734-treated cohorts: 1 mg: −57%, 5 mg: −66%, 15 mg: −70%, 50 mg: −82%, and 150 mg: −90%, versus +7.5% for the placebo-treated group.

Increasing doses were generally associated with a longer duration of pDC reduction. The effect was reversible in all cases. As shown in FIGS. 4-6, median pDC levels returned to above 50% of baseline at the following timepoints for each cohort: 1 mg: Day 29, 5 mg: Day 57, 15 mg: Day 57, 50 mg: Day 85, 150 mg: Day 113. Thus, subcutaneous injection of VIB7734 in doses ranging from 1 to 150 mg caused a reversible, dose-dependent reduction in circulating pDC levels.

The maximal degree of reduction from baseline of the median pDC level was −90%. The increase in the maximal depletion appears to nearly plateau at doses of 15 to 150 mg, suggesting that doses higher than 150 mg are unlikely to cause a greater degree of maximal depletion.

### Example 6: SAD Study: Pharmacodynamic Evaluations (Type I IFNGS)

Whole blood was collected on screening and Days 1, 2, 4, 8, 15, 29, 57, and 85 to measure the expression of mRNA for certain types of type I IFN-inducible genes using a 21-gene assay. The type I IFNGS was determined by assaying the mRNA levels of 21 type I IFN-inducible genes in a biological sample taken from the subjects, determining an average value (mean or median) of the mRNA levels of 21 type I IFN-inducible gene, normalizing the average value against an average of mRNA levels of 3 housekeeping genes (18S rRNA, β actin, and GAPDH), and obtaining a composite outcome. The type I IFNGS was reported by two methods, “median fold change” and “median target neutralization.” The first method, called “median fold change” is the fold difference in levels of the gene products when compared to healthy controls, which are normalized to 1. Thus, for a subject, a median fold change of 4 indicates that the type I IFN-inducible gene products are 4 times higher than for healthy controls. The median fold change for each cohort at each visit are presented in Table 2.

TABLE 2
Biomarkers Results - As Treated Population (type I IFNGS fold Change).

| | | VIB7734 | VIB7734 | VIB7734 | VIB7734 | VIB7734 | VIB7734 |
|---|---|---|---|---|---|---|---|
| | Placebo | 1 mg | 5 mg | 15 mg | 50 mg | 150 mg | Total |
| Characteristics | N = 10 | N = 3 | N = 6 | N = 6 | N = 6 | N = 5 | N = 26 |
| Baseline (n) | 9 | 3 | 6 | 6 | 6 | 5 | 26 |
| Median | 2.130 | 17.800 | 1.255 | 2.880 | 3.705 | 0.800 | 1.995 |
| (Min, Max) | (0.78, | (4.06, | (0.74, | (0.88, | (0.70, | (0.44, | (0.44, |
| | 23.59) | 61.62) | 12.47) | 9.78) | 10.39) | 20.58) | 61.62) |

Percent Change from Baseline (%)

| | | | | | | | |
|---|---|---|---|---|---|---|---|
| Day 4 (n) | 8 | 3 | 6 | 6 | 6 | 5 | 26 |
| Median | 0.918 | −5.858 | −12.698 | −11.330 | −14.310 | 8.649 | −7.443 |
| (Min, Max) | (−32.64, | (−19.78, | (−57.42, | (−72.84, | (−67.77, | (−52.90, | (−72.84, |
| | 13.48) | −1.23) | 52.89) | 7.14) | −0.72) | 65.91) | 65.91) |

Percent Change from Baseline (%)

| | | | | | | | |
|---|---|---|---|---|---|---|---|
| Day 8 (n) | 9 | 3 | 6 | 6 | 6 | 5 | 26 |
| Median | −11.702 | 4.607 | −12.038 | −45.077 | −25.529 | −12.634 | −15.218 |
| (Min, Max) | (−49.59, | (−17.17, | (−64.15, | (−69.44, | (−85.88, | (−74.37, | (−85.88, |
| | 544.91) | 27.59) | 23.14) | 61.36) | 4.95) | 127.27) | 127.27) |
| Day 29 (n) | 9 | 3 | 6 | 6 | 5 | 5 | 25 |
| Median | 3.846 | 12.865 | 13.814 | −51.869 | −25.000 | −10.739 | −10.739 |
| (Min, Max) | (−42.56, | (−8.75, | (−39.29, | (−78.64, | (−90.28, | (−21.92, | (−90.28, |
| | 69.23) | 30.79) | 42.15) | 7.95) | 14.85) | 79.55) | 79.55) |

Percent Change from Baseline (%)

| | | | | | | | |
|---|---|---|---|---|---|---|---|
| Day 57 (n) | 9 | 3 | 6 | 6 | 6 | 5 | 26 |
| Median | −4.959 | 0.281 | 8.756 | −17.887 | −14.218 | 1.020 | −9.464 |
| (Min, Max) | (−45.65, | (−17.61, | (−39.80, | (−78.33, | (−86.71, | (−55.68, | (−86.71, |
| | 31.28) | 48.03) | 96.15) | 23.86) | 5.63) | 70.45) | 96.15) |

Percent Change from Baseline (%)

| | | | | | | | |
|---|---|---|---|---|---|---|---|
| Day 85 (n) | 9 | 3 | 6 | 5 | 6 | 5 | 25 |
| Median | 4.239 | −9.360 | −8.155 | −23.768 | −8.003 | 8.219 | −16.861 |
| (Min, Max) | (−32.55, | (−19.33, | (−60.22, | (−72.62, | (−80.56, | (−84.47, | (−84.47, |
| | 96.51) | 3.05) | 551.35) | −13.24) | 29.80) | 77.27) | 551.35) |

IFN = interferon;
Max = maximum;
Min = minimum;
N = number of subjects;
n = subset of N

The second metric, “median target neutralization,” is a measure of the percentage of the level of the gene products compared to the baseline result, which is normalized to 100%. This is useful for comparing change over time. For example, a median target neutralization ratio at a visit of 30% means that the type I IFN-inducible gene products are 30% of the level of what they were at baseline.

Table 3 shows the median target neutralization ratio by cohort and visit for the subgroup of subjects who had an elevated baseline type I IFN signature. The median neutralization ratio for the IFN-high subgroup was less than 100% for all VIB7734-treated groups at Day 4 (first timepoint measured after dosing), compared to 100% for the placebo group. Median neutralization ratio for the IFN-high subgroup was at its lowest at the Day 8 visit (37.9% for all VIB7734-treated vs. 118% for placebo. Median target neutralization of the IFN-high subgroup remained <100% for all VIB7734-treated cohorts at all timepoints with the exception of the lowest dose cohort (1 mg) which was >100% of baseline at the Day 8, 29, and 57 visits.

TABLE 3
Biomarker Results - As Treated Population (Neutralization
Ratio of the Type I IFN signature).

| | | VIB7734 | VIB7734 | VIB7734 | VIB7734 | VIB7734 | VIB7734 |
|---|---|---|---|---|---|---|---|
| | Placebo | 1 mg | 5 mg | 15 mg | 50 mg | 150 mg | Total |
| Characteristics | N = 10 | N = 3 | N = 6 | N = 6 | N = 6 | N = 5 | N = 26 |
| Baseline (n) | 0 | 0 | 0 | 0 | 0 | 0 | 0 |
| Median | NA | NA | NA | NA | NA | NA | NA |
| (Min, Max) | (NA, | (NA, | (NA, | (NA, | (NA, | (NA, | (NA, |
| | NA) | NA) | NA) | NA) | NA) | NA) | NA) |
| Day 4 (n) | 3 | 3 | 1 | 3 | 3 | 2 | 12 |
| Median | 100.20 | 87.39 | 40.18 | 43.03 | 39.58 | 83.60 | 63.59 |
| (Min, Max) | (79.95, | (80.53, | (40.18, | (26.42, | (31.20, | (47.74, | (26.42, |
| | 105.61) | 98.36) | 40.18) | 120.64 | 79.45) | 119.46) | 120.64) |
| Day 8 (n) | 4 | 3 | 1 | 3 | 3 | 2 | 12 |
| Median | 117.79 | 101.33 | 35.76 | 31.69 | 17.38 | 57.24 | 37.91 |
| (Min, Max) | (101.92, | (69.56, | (35.76, | (30.64, | (16.31, | (30.85, | (16.31, |
| | 945.43) | 104.26) | 35.76) | 40.07) | 75.28) | 83.63) | 104.26) |
| Day 29 (n) | 4 | 3 | 1 | 3 | 3 | 2 | 12 |
| Median | 100.54 | 101.78 | 83.83 | 30.72 | 12.01 | 81.23 | 75.11 |
| (Min, Max) | (64.10, | (87.90, | (83.83, | (18.82, | (10.90, | (75.71, | (10.90, |
| | 198.41) | 109.41) | 83.83) | 31.60) | 74.51) | 86.75) | 109.41) |
| Day 57 (n) | 3 | 3 | 1 | 3 | 3 | 2 | 12 |
| Median | 103.94 | 114.88 | 75.26 | 27.89 | 47.60 | 65.65 | 75.52 |
| (Min, Max) | (59.24, | (75.78, | (75.26, | (27.40, | (12.25, | (40.17, | (12.25, |
| | 111.57) | 126.08) | 75.26) | 80.92) | 116.46) | 91.12) | 126.08) |
| Day 85 (n) | 4 | 3 | 1 | 3 | 3 | 2 | 12 |
| Median | 129.87 | 89.45 | 40.99 | 41.71 | 84.35 | 51.13 | 77.36 |
| (Min, Max) | (75.21, | (70.36, | (40.99, | (30.84, | (19.53, | (17.09, | (17.09, |
| | 176.79) | 94.26) | 40.99) | 94.85) | 159.89) | 85.17) | 159.89) |

IFN = interferon;
Max = maximum;
Min = minimum;
N = number of subjects;
n = Subset of N

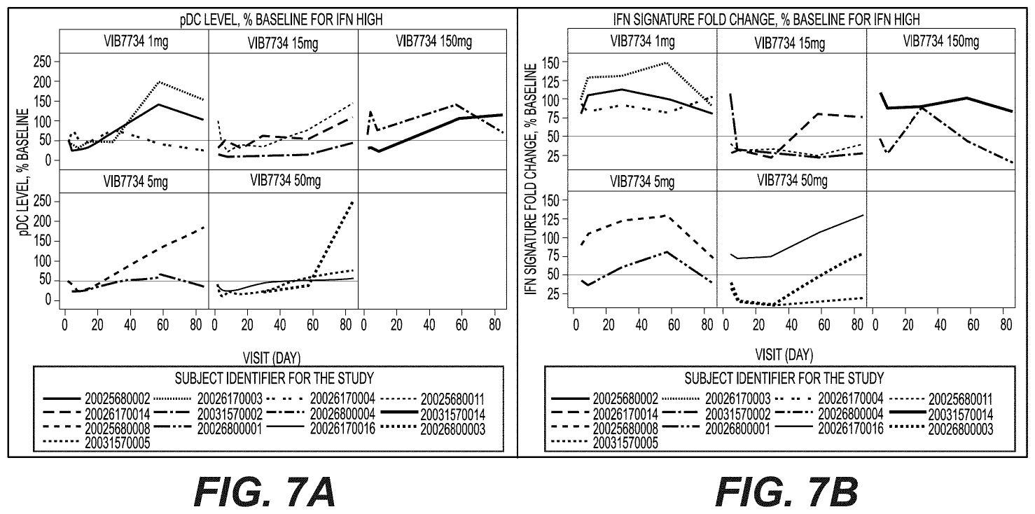

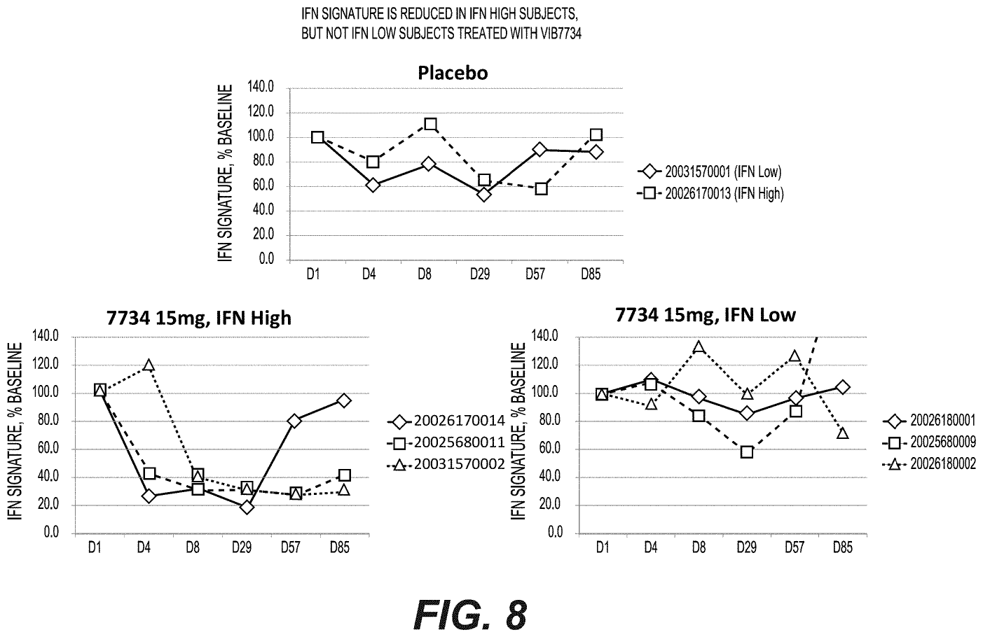

FIG. 7 shows the % of baseline IFN signature fold change over time for each subject within the cohorts with elevated baseline type 1 IFNGS. In a majority of subjects with an elevated type I IFNGS, reduction in pDC levels (FIG. 7A) correlated with a reduction in type I IFNGS (reported as % of baseline fold change) (FIG. 7B).

### Multiple Ascending Dose (MAD) Study

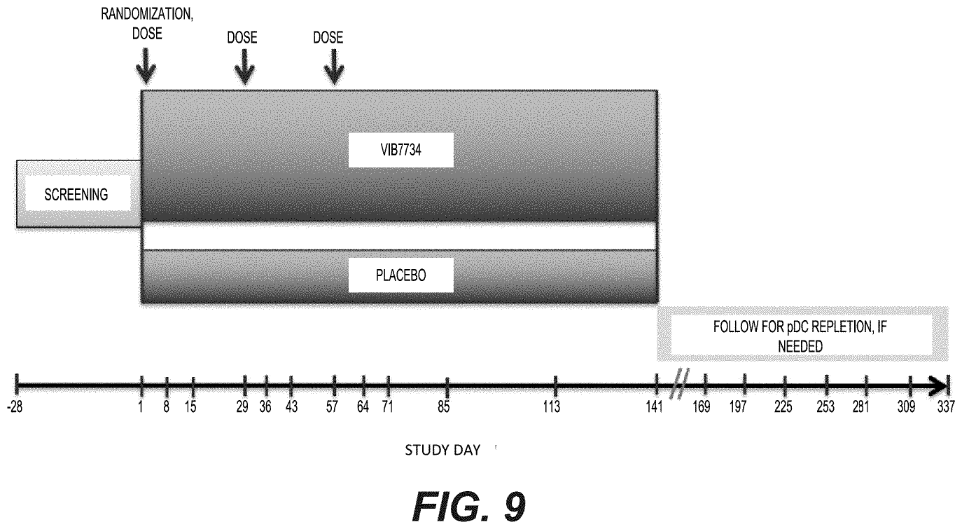

A Phase Ib, randomized, double-blinded (sponsor and site pharmacist were unblinded), placebo-controlled study was carried out to evaluate the safety and tolerability of multiple-ascending subcutaneous (SC) doses of VIB7734 when added to standard of care in subjects with at least one of the following autoimmune diseases: systemic lupus erythematosus (SLE), cutaneous lupus erythematosus (CLE), systemic sclerosis, polymyositis, and dermatomyositis (FIG. 9). The study also evaluated the efficacy of VIB7734 on cutaneous lupus activity. The rationale for this study was: (1) to evaluate the safety, PK, PD, and immunogenicity of multiple doses of VIB7734 in a relevant subject population, (2) to evaluate whether VIB7734 can improve skin manifestations of lupus, and (3) to evaluate the effect of VIB7734 on the level of pDCs and type I IFNGS in the skin. A SC route of administration was selected for this study to provide the most relevant information for future studies that will use a SC formulation. The dose regimens tested were VIB7734 5 mg, 50 mg, or 150 mg SC q4 weeks or placebo SC q4 weeks. The PK/PD model from the single dose study indicates that a dose in the range of 50 to 100 mg SC every 4 weeks is the minimal necessary dose to provide continuous near-maximal pDC reductions. The dose of 5 mg SC q4 weeks (Cohort 1) was expected to provide a submaximal PD effect that would help define the minimum necessary dose for this drug to achieve optimal PD effect. The dose of 50 mg SC q4 weeks (Cohort 2) was chosen to evaluate a dose in the range of the expected target dose. The 10-fold increase in dose between Cohorts 1 and 2 is justified by (a) the predicted difference in maximal pDC reduction between the two dose cohorts is relatively small (−78% versus −87%), (b) the single-dose study with VIB7734 did not demonstrate any safety concerns for all doses tested (maximum dose of 150 mg SC), and (c) the safety margin is high for all doses tested in this study. The dose of 150 mg SC q4 weeks tested the upper end of the range of potential doses of VIB7734 that could be needed, particularly if a higher dose is needed to deplete pDCs in target tissues than in the circulation. The study enabled refinement of the PK/PD model and enabled selection of one or more doses to be tested in later phase trials.

### Example 7: MAD Study Design

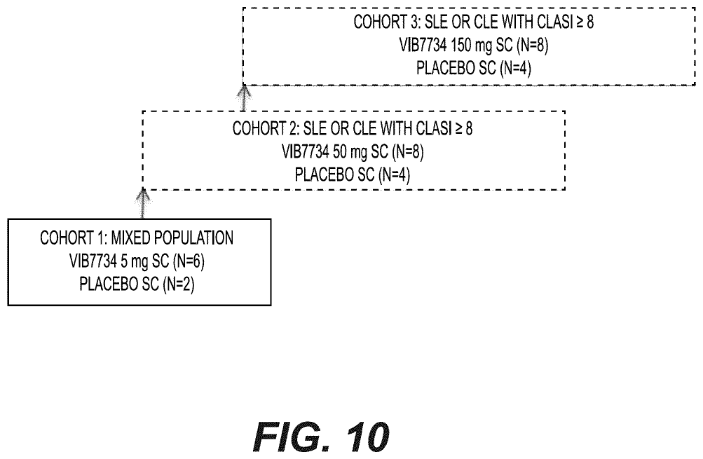

A total of 31 adult subjects were enrolled in 3 sequential cohorts, with 8 subjects in Cohort 1, 12 subjects in Cohort 2, and 11 subjects in Cohort 3. Subjects were maintained on their standard of care treatment and an ILT7-binding protein (VIB7734) or placebo administration was performed in addition to this treatment. Randomization was not stratified. Cohorts were enrolled sequentially (FIG. 10) to allow for review of safety and tolerability data before progression to the next cohort. In Cohort 1, subjects were randomized in a 3:1 ratio to receive VIB7734 or matching placebo by SC injection every 4 weeks for a total of 3 doses. The dose of VIB7734 administered was 5 mg SC q4 weeks for 3 doses. In Cohorts 2 and 3, subjects were randomized in a 2:1 ratio to receive VIB7734 50 mg (Cohort 2) or 150 mg (Cohort 3) or matching placebo by SC injection q4 weeks for 3 doses.
Cohort 1: VIB7734, 5 mg (n=6) or placebo (n=2) injected SC every 28 days×3 doses;
Cohort 2: VIB7734, 50 mg (n=8) or placebo (n=4) injected SC every 28 days×3 doses;
Cohort 3: VIB7734, 150 mg (n=8) or placebo (n=3) injected SC every 28 days×3 doses.

Cohort 1 enrolled a mixed disease population, comprising subjects with systemic lupus erythematosus (SLE) or Sjogren's syndrome with no minimum disease activity requirement, to evaluate the safety profile of multiple doses of VIB7734 in subjects across various pDC-driven indications. Cohorts 2 and 3 recruited active SLE or cutaneous lupus erythematosus (CLE) patients with a CLASI Activity score (CLASI-A) of ≥8 so that efficacy could be explored at the doses tested in these cohorts, and the effect on skin biopsy specimens could be examined. A trained physician-investigator for designee performed the CLASI-A assessment. Whenever possible, the same rater evaluated a subject throughout the study. Sites also designated a backup rater for each subject.

The screening procedures for Cohorts 2 and 3 include taking digital photographs of the existing skin lesions at Days 29, 57 and 85. The goals of the skin photography were 1) to confirm that the subject has skin lesions consistent with lupus as part of the screening procedures, and 2) to provide visual evidence of the effect of the drug on skin lesions. The investigator or sub-investigator who examined the patient at screening decided on the anatomic areas to be photographed based on the location of the lesions. These anatomic areas were documented in the screening eCRF in sufficient detail so that the same areas could be photographed at additional time points (Days 113 and 145). Lighting was maintained as consistent as possible at all photography visits. The photographs were uploaded and reviewed by a central reviewer to confirm that skin lesions consistent with lupus were present.

The multiple dose study had three study periods: screening, treatment period, and extended follow-up for pDC repletion (FIG. 9). A screening visit was performed within 28 days prior to dosing. Randomized subjects received VIB7734 on Days 1, 29, and 57. For all administrations, VIB7734 was administered in the clinic and the subject were observed for at least 90 minutes after dosing. Subjects were followed until at least Day 141. After the Day 141 visit, subjects were notified whether they have met the protocol definition for an adequate pDC level. The protocol definition for an adequate pDC level is either >50% of the subject's baseline value or >0.036% of peripheral blood mononuclear cells (PBMCs). If a subject's pDC level meets the criteria for an adequate level, the subject exited the study. If a subject did not met criteria for an adequate pDC level at the Day 141 visit, the subject was informed and was asked to continue to return for pDC follow-up visits until the subject met the protocol definition for an adequate pDC level or the Day 337 visit was reached. pDC follow-up visits occurred regardless of whether the subject received VIB7734 or placebo, and the subject and site was not unblinded until the study had ended.

An unblinded Dose Escalation Committee (DEC) reviewed the safety of each dose cohort according to pre-specified criteria to determine if it is safe to escalate to the next dosing cohort. For escalation from Cohort 1 to 2, the DEC reviewed the cumulative safety data after the 8th subject in Cohort 1 has completed the Day 15 visit (or when the last evaluable Cohort 1 subject reaches Day 15 if the 8th subject withdraws prior to Day 15). For escalation from Cohort 2 to 3, the DEC reviewed the cumulative safety data once 9 Cohort 2 subjects have completed the Day 15 visit. If the data from any cohort were inadequate to decide about the safety of escalating to the next cohort, then the sponsor could elect to hold escalation pending reassessment of the cohort at a later timepoint.

### Example 8: Evaluation of Adverse Reactions

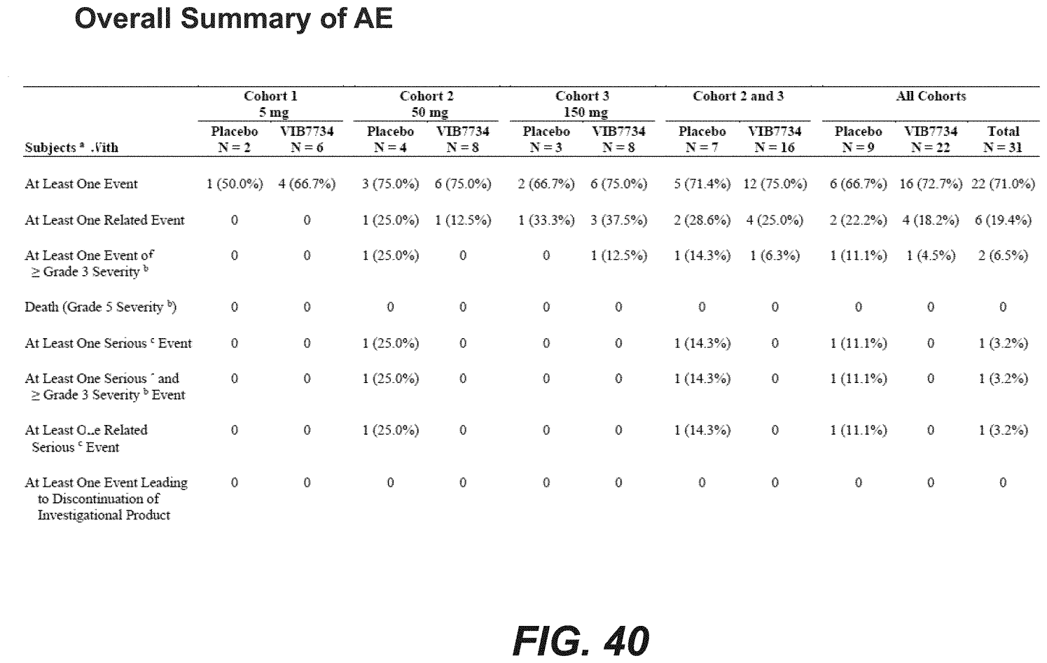

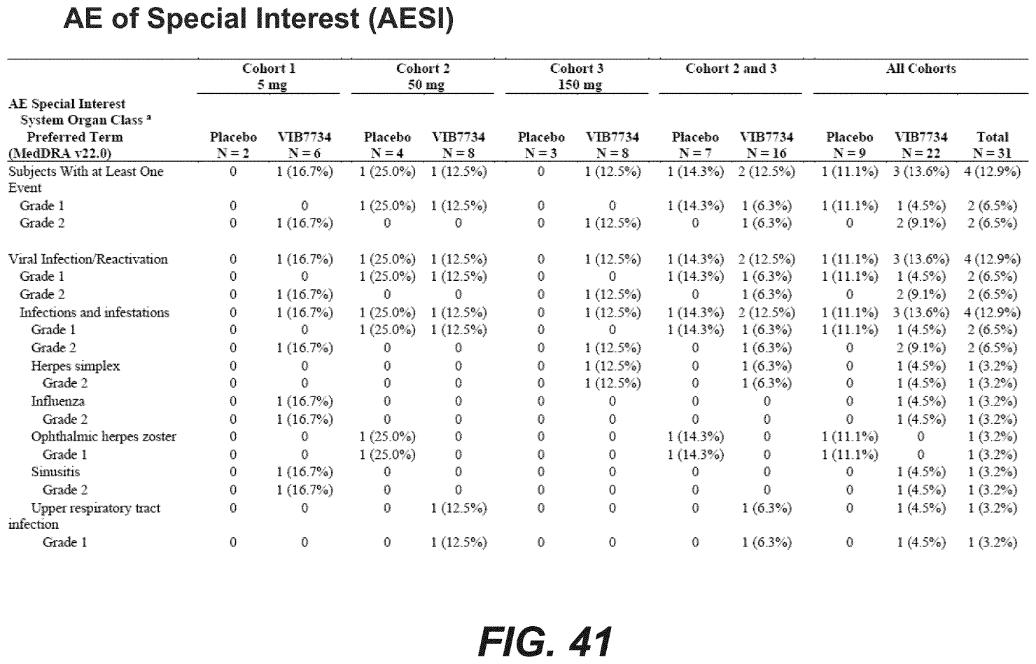

The safety and tolerability of VIB7734 was measured by the incidence of treatment-emergent adverse events (TEAEs), adverse events of special interest (AESIs), and treatment-emergent serious adverse events (TESAEs). Laboratory measurements, vital sign measurements, and ECG parameters were also evaluated as part of safety. Adverse event (AE) and serious adverse event (SAE) collection began after the subject signed the informed consent document and lasted until the final visit. TEAEs were defined as any AE that occurs after dosing on or after the day of first administration of VIB7734 through the end of follow-up. All AEs were required to be coded by the Medical Dictionary for Regulatory Activities (MedDRA). All SAEs were required to be reported, whether or not considered causally related to VIB7734, or to the study procedures. TEAEs, TESAEs, and TEAESIs were required to be summarized overall, as well as categorized by MedDRA System Organ Class and Preferred Term, by severity, and by relationship to VIB7734. AESIs were also required to be recorded within 24 hours of knowledge of the event on the eCRF, even if the event is non-serious. No AEs (FIG. 40) or AESIs (FIG. 41) were observed in VIB7734-treated subjects in Cohorts 1, 2, and 3. The proportion of subjects with an adverse event was similar in the VIB7734 and placebo groups (about 73% vs. about 67%, respectively; FIG. 41).

### Example 9: Immunogenicity Evaluations

Blood samples were collected on Days 1, 29, 57, 85, 113, and 141, and, if applicable, on Days 197, 253, and 309 to evaluate anti-drug antibody (ADA) response to VIB7734 in human serum. ADA status and titer were summarized by treatment group. These evaluations were performed utilizing a validated immunoassay method. No ADA response to VIB7734 in human serum was observed in subjects in Cohorts 1,2 or 3 (data not shown).

### Example 10: Pharmacokinetic Evaluations

Blood samples were collected on Days 1, 8, 15, 29, 36, 43, 57, 64, 71, 85, 113, and 141, and, if applicable, on Days 169, 197, 225, and 253 to evaluate PK of VIB7734 in human serum. The PK of VIB7734 in serum was measured utilizing a validated immunoassay method. Specific procedures for sample collection, processing, storage, and shipment can be found in a separate laboratory manual provided to the sites. Non-compartmental analysis were performed for VIB7734-treated subjects. VIB7734 concentration-time profiles were generated.

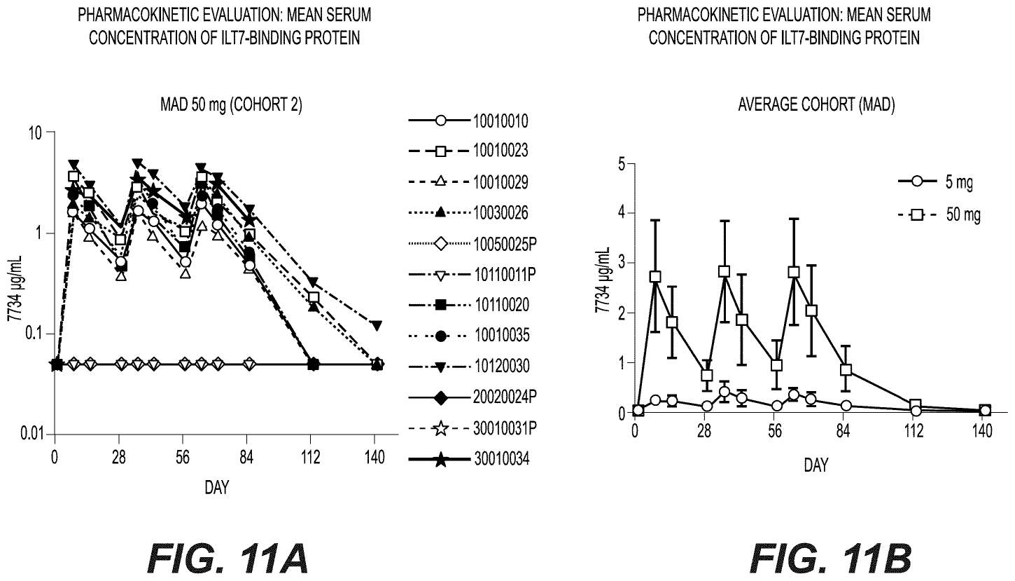

FIG. 11 shows the serum concentration profile of VIB7734 following multiple subcutaneous doses (every 4 weeks for 3 doses) of 5 mg (Cohort 1) or 50 mg (Cohort 2) of VIB7734. FIG. 11A shows the serum concentration profile of VIB7734 in Cohort 2 subjects. FIG. 11B shows the mean serum concentration profile of VIB7734 in subjects in Cohort 1 (solid circles) and Cohort 2 (solid squares). As shown in FIG. 11B, the mean serum concentrations of VIB7734 were increased in Cohort 2 subjects compared to Cohort 1 subjects throughout Day 112.

### Example 11: Pharmacodynamic Evaluations: Circulating pDC Levels

The effect of VIB7734 on circulating pDC levels was evaluated in blood using flow cytometry. Changes from baseline were described and level as a percent of baseline was summarized. Whole blood samples for pDC levels (all subjects) and for CD19+ B cells (at screening, for subjects previously administered rituximab, ocrelizumab, ofatumumab, or an experimental B-cell-depleting mAb) were collected on Days 1, 8, 15, 29, 36, 43, 57, 64, 71, 85, 113, and 141. The baseline pDC level is defined as the average of the levels measured at the Screening visit and the Day 1 (predose) level. If the screening (Visit 1) sample is not drawn or fails for technical reasons, it must be repeated and results must be available before the subject can be randomized since the result is needed to determine that the subject meets all inclusion/exclusion criteria. If only one value was available, then this value was used as the baseline.

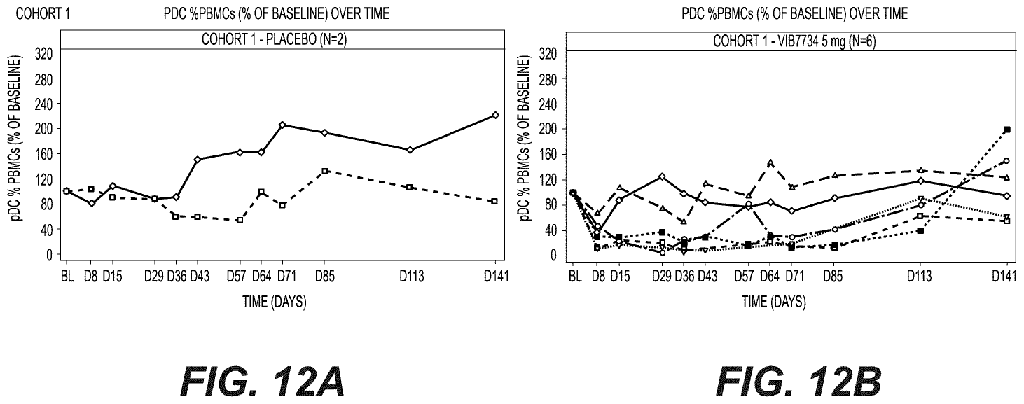

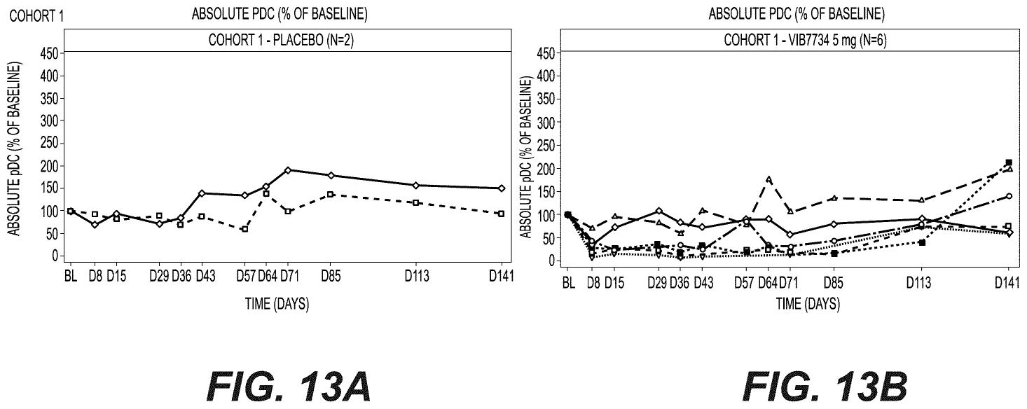

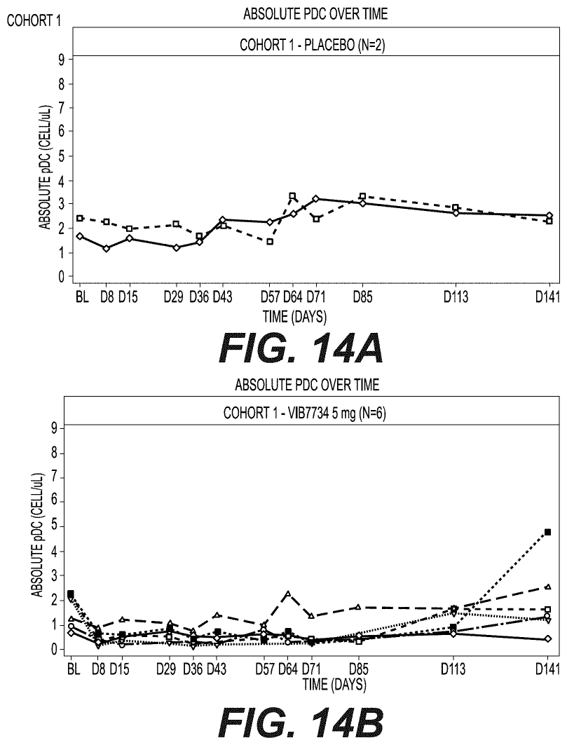

The levels and change from baseline in pDCs (% of PBMC cells) over time in whole blood of subjects in Cohort 1 is presented in FIG. 12. The levels and change from baseline in absolute concentration of pDCs over time in in whole blood of subjects in Cohort 1 is presented in FIG. 13. The changes in absolute pDC levels over time in in whole blood of subjects in Cohort 1 is presented in FIG. 14. Consistent with the results in the single ascending dose study, there was a decrease in the blood level of pDCs in subjects in Cohort 1 treated with multiple SC doses (every 4 weeks for 3 doses) of 5 mg of VIB7734 (FIGS. 12B, 13B and 14B), compared to subjects treated with placebo (FIGS. 12A, 13A and 14A).

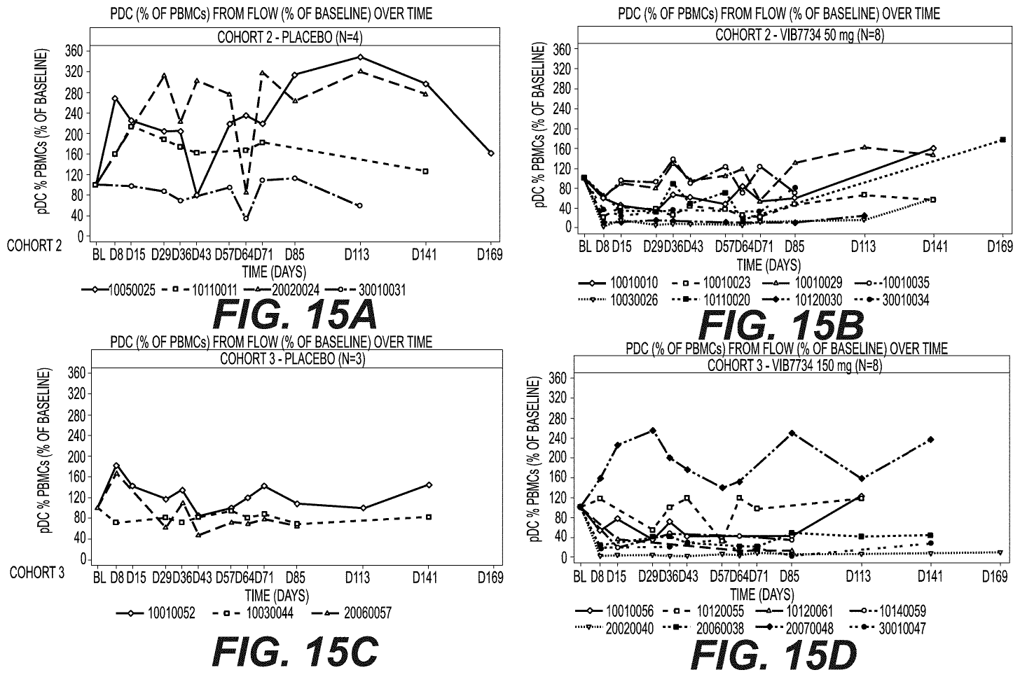

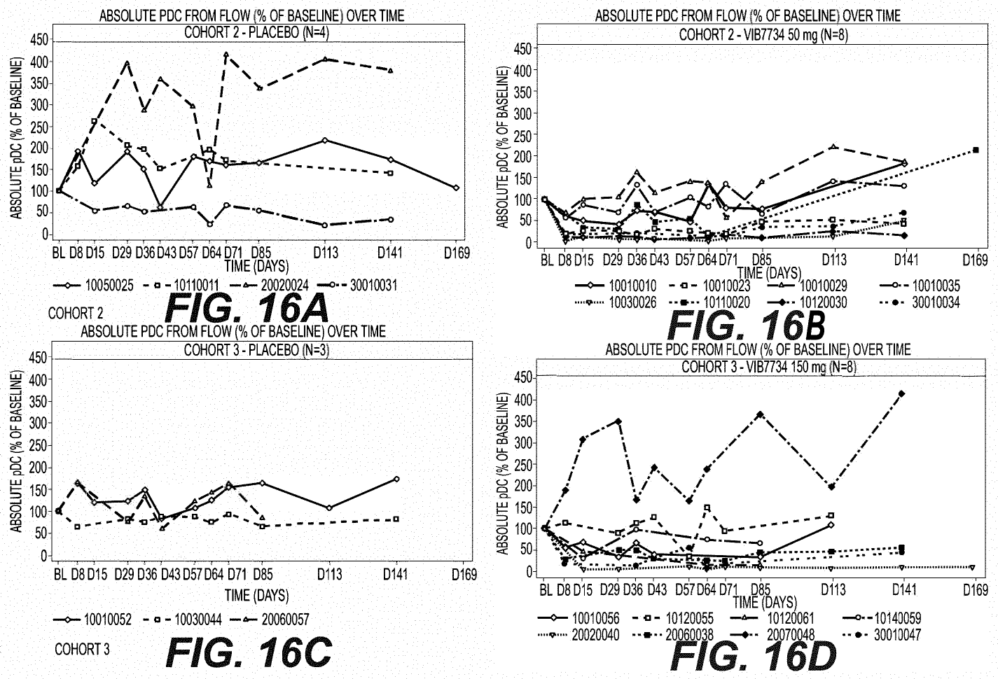

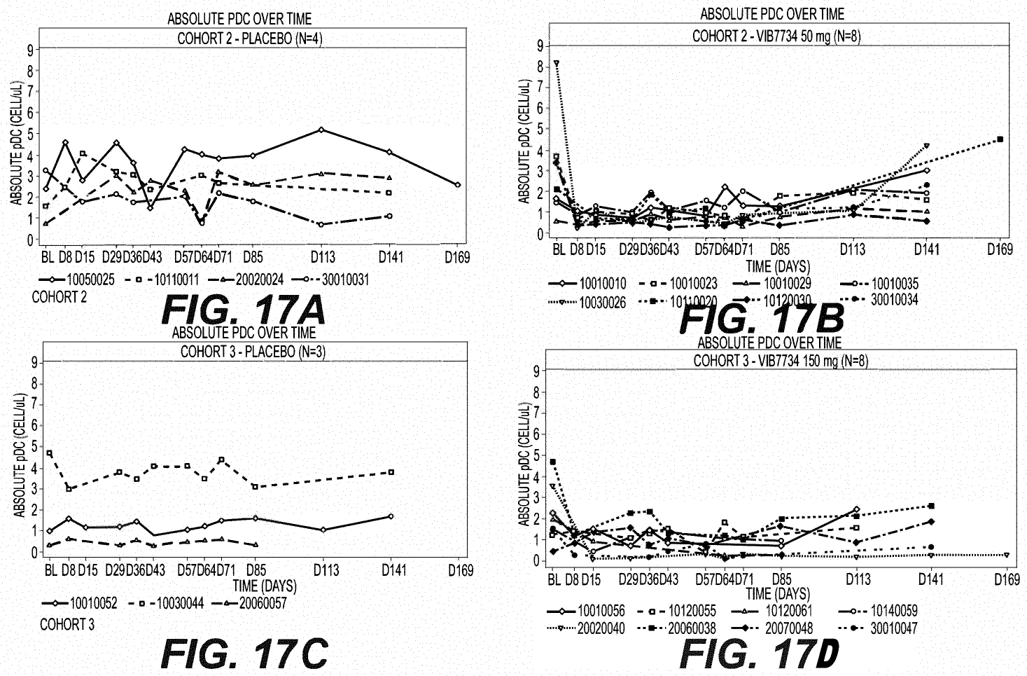

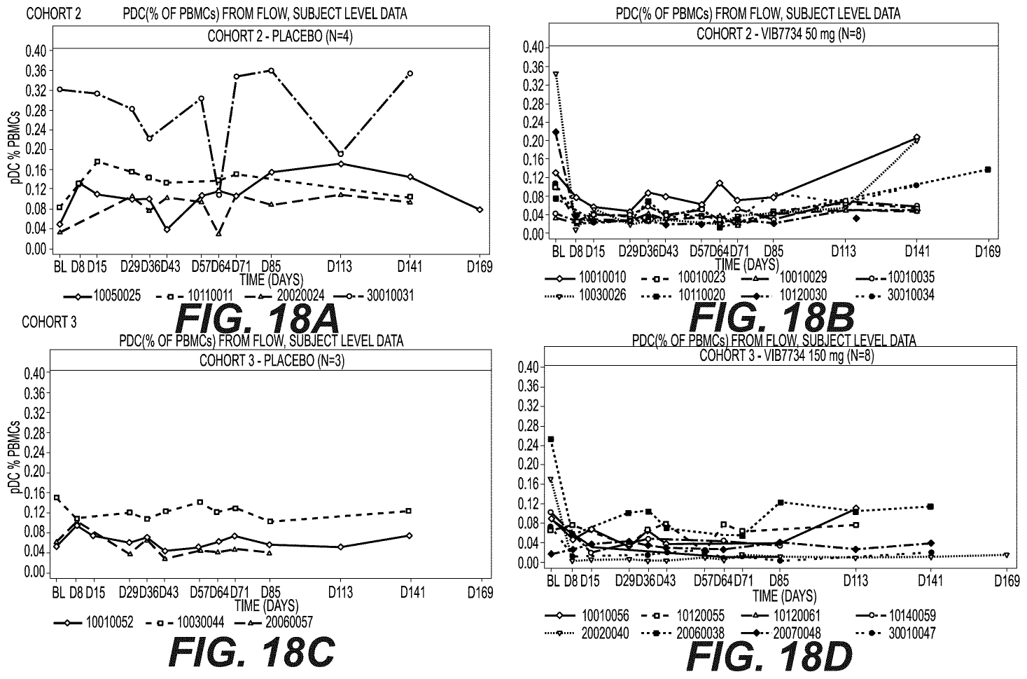

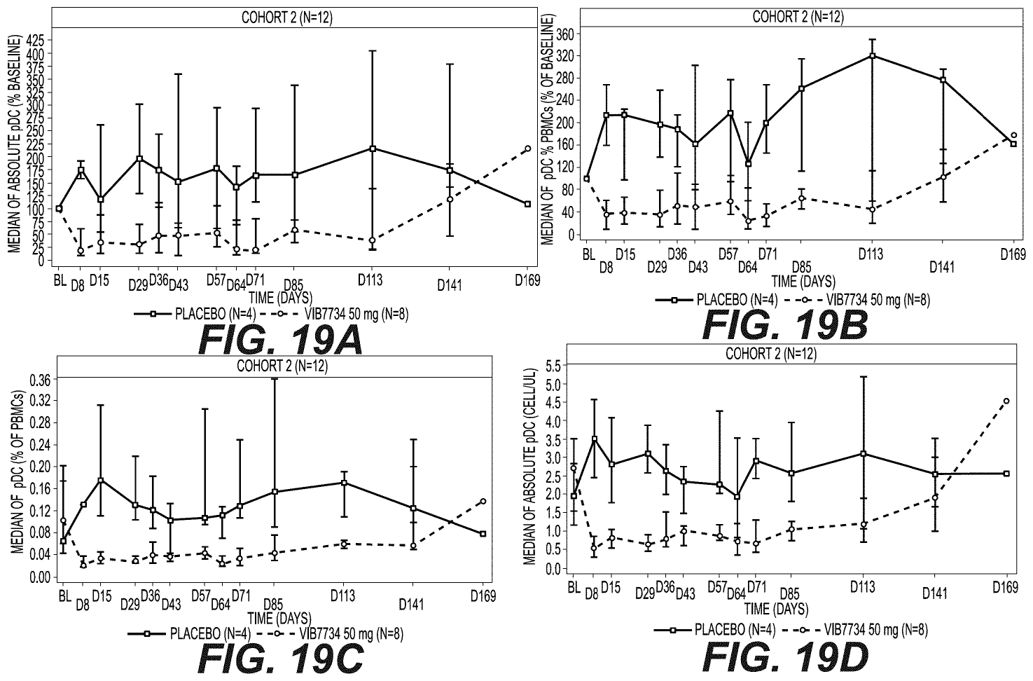

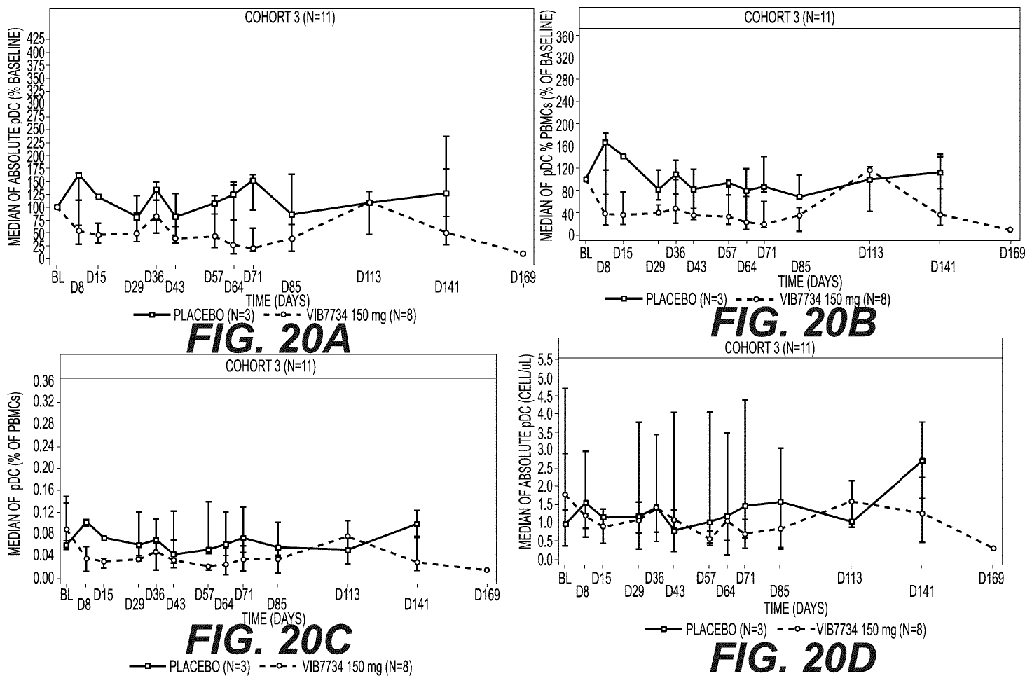

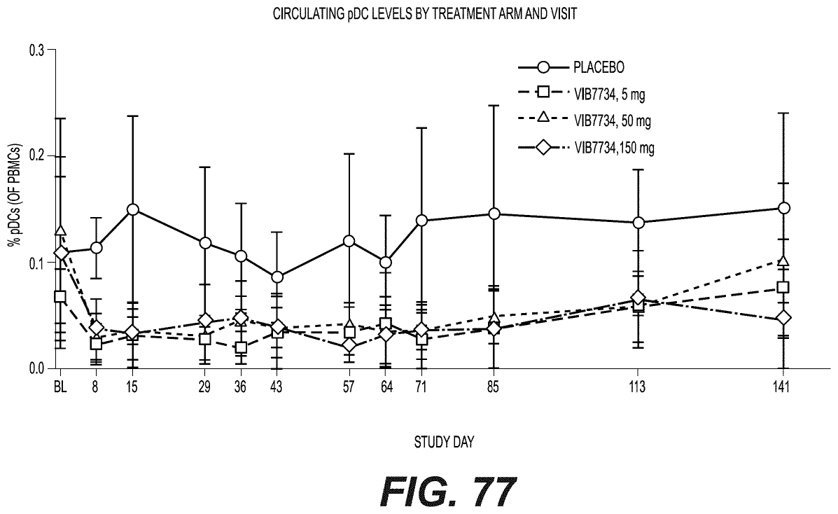

The changes in pDCs levels (%) over time, as a percent of the baseline level (value using % peripheral blood mononuclear cells) in whole blood of subjects in Cohort 2 and Cohort 3 is presented in FIG. 15. The levels and change from baseline in absolute concentration of pDCs over time in in whole blood of subjects in Cohort 2 and Cohort 3 is presented in FIG. 16. The changes in absolute pDC levels over time in in whole blood of subjects in Cohort 2 and Cohort 3 is presented in FIG. 17. The levels and change from baseline in pDCs (as % of PBMC cells) over time in whole blood of subjects in Cohort 2 and Cohort 3 is presented in FIG. 18. Similar to subjects in Cohort 1, there was a decrease in the blood level of pDCs in subjects in Cohort 2 treated with multiple SC doses (every 4 weeks for 3 doses) of 50 mg of VIB7734 (FIGS. 15B, 16B, 17B and 18B), compared to subjects treated with a placebo (FIGS. 15A, 16A, 17A and 18A). Similarly, there was a decrease in the blood level of pDCs in most subjects in Cohort 3 treated with multiple SC doses (every 4 weeks for 3 doses) of 150 mg of VIB7734 (FIGS. 15D, 16D, 17D and 18D), compared to subjects treated with placebo (FIGS. 15C, 16C, 17C and 18C). Additionally, as shown in FIGS. 19A-D, the median of pDC levels over time in whole blood of subjects in Cohort 2 following multiple SC doses (every 4 weeks for 3 doses) of 50 mg of VIB7734 showed a marked decrease compared to the median of pDC levels over time in whole blood of Cohort 2 subjects treated with a placebo. Similarly, as shown in FIGS. 20A-D, the median of pDC levels over time in whole blood of subjects in Cohort 3 treated with multiple SC doses of 150 mg of VIB7734 showed a marked decrease compared to the median of pDC levels over time in whole blood of Cohort 3 subjects treated with a placebo. As further shown in FIG. 77, reductions in the median of circulating pDC levels (measured as % PBMC cells) were evident at week 1 and persisted through at least Day 85 in VIB7734-treated subjects in Cohort 1 (treated with 5 mg of VIB7734), Cohort 2 (treated with 50 mg of VIB7734) and Cohort 3 (treated with 150 mg of VIB7734), compared to the median of circulating pDC levels in placebo-treated subjects.

### Example 12: Pharmacodynamic Evaluations: Type I IFN Signature in Blood

The type I IFNGS in the skin and blood was measured using the 21-gene test. The effect of VIB7734 on the type I IFNGS in blood was evaluated in Cohorts 1, 2, and 3. Whole blood was collected at Days 1, 8, 15, 29, 43, 57, 71, 85, 113, and 141 in PAXgene tubes to measure the overexpression of mRNA for certain types of type I IFN-inducible genes. Any remaining RNA isolated from the samples was used for additional analytical studies of changes in gene expression. High type I IFNGS levels were present at baseline in whole blood of 18 of 23 subjects (78%) in cohorts 2 and 3.

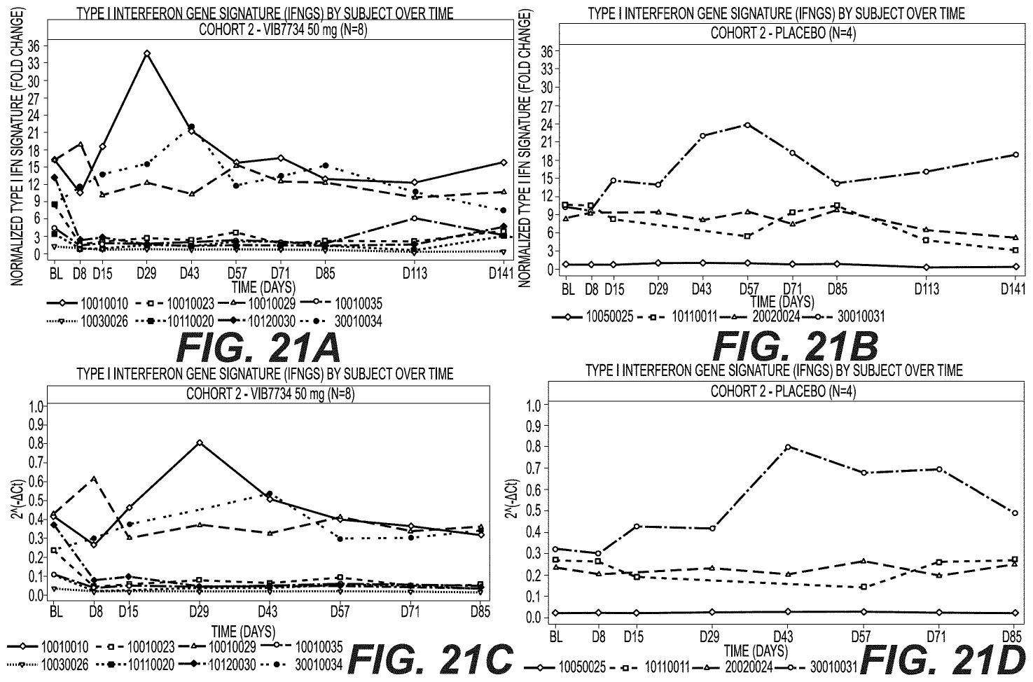

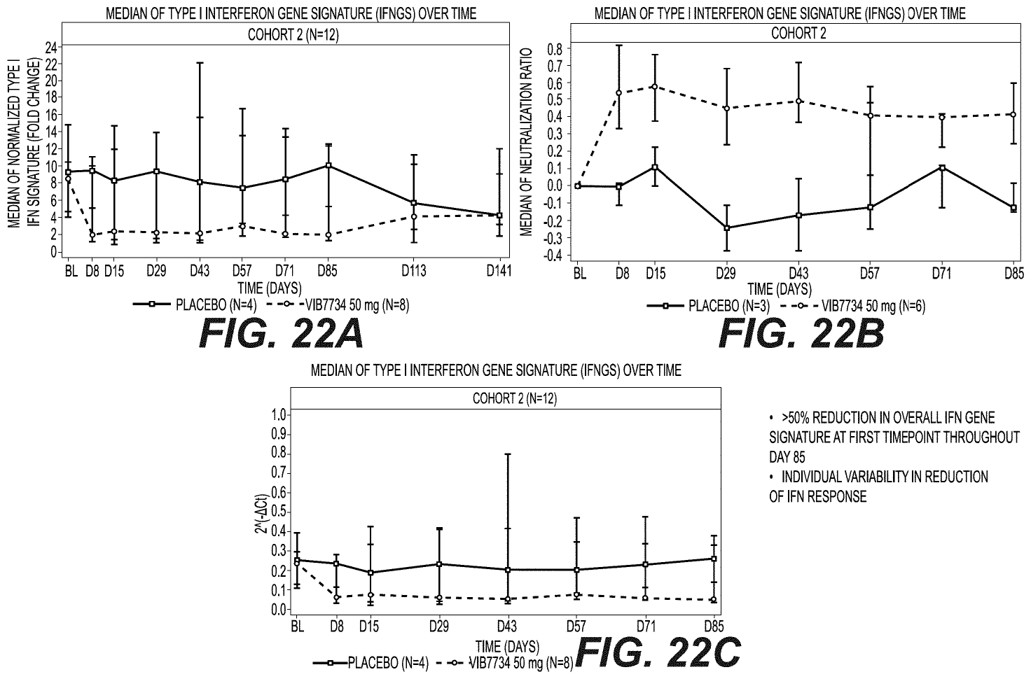

FIG. 21 shows type I IFNGS levels (measured as fold change (FIGS. 21A and 21B or absolute score (FIGS. 21C and 21D)) over time in whole blood of subjects in cohort 2 treated with 50 mg of VIB7734. There was a decrease in the blood level of Type I IFNGS in subjects in Cohort 2 treated with multiple SC doses (every 4 weeks for 3 doses) of 50 mg of VIB7734 (FIGS. 21A and 21C), compared to subjects treated with placebo (FIGS. 21B and 21D). Also, as shown in FIGS. 22A and 22C, the median of type I IFNGS levels (measured as fold change and absolute score, respectively) over time in whole blood of subjects in cohort 2 treated with multiple SC doses (every 4 weeks for 3 doses) of 50 mg of VIB7734 showed a marked decrease compared to the median of type I IFNGS levels over time in whole blood of subjects in cohort 2 treated with placebo. As shown in FIG. 22C, there was a greater than 50% reduction in overall type I IFNGS (measured as absolute score) at first time point throughout Day 85 for the VIB7734-treated Cohort 2 subjects. Further, as expected, the median of type I IFNGS levels (measured as neutralization ratio) over time in whole blood of subjects in cohort 2 treated with multiple SC doses (every 4 weeks for 3 doses) of 50 mg of VIB7734 increased compared to the median of pDC levels over time in whole blood of subjects in cohort 2 treated with placebo (FIG. 22B).

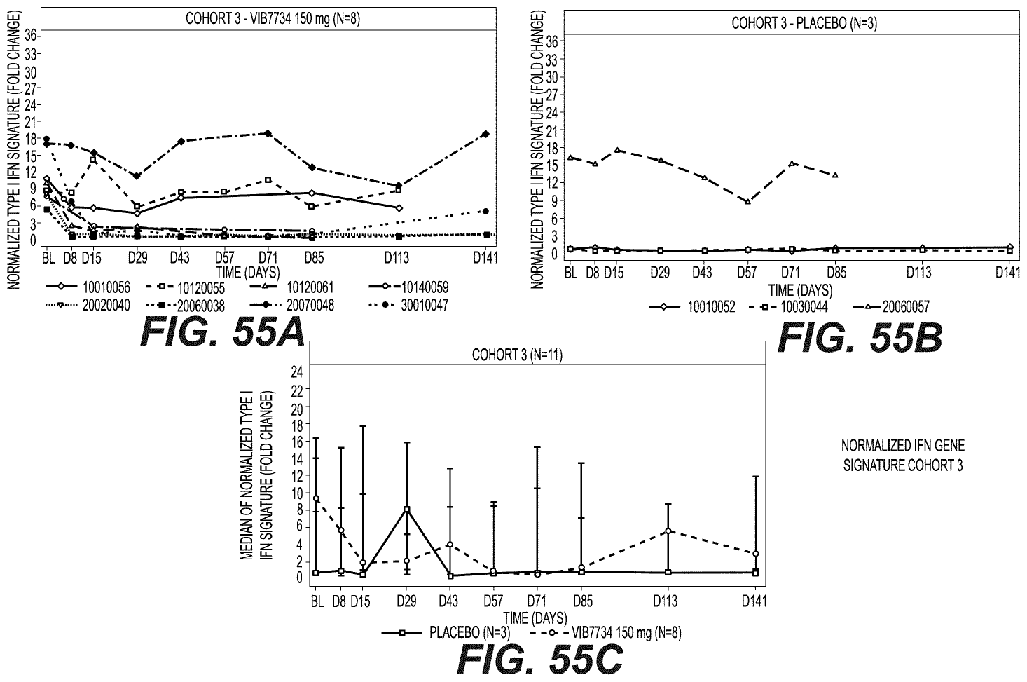

FIG. 55 shows normalized type I IFNGS levels (measured as fold change) over time in whole blood of subjects in Cohort 3 following multiple subcutaneous doses (every 4 weeks for 3 doses) of 150 mg of VIB7734 or a placebo. There was a decrease in the blood level of normalized Type I IFNGS levels over time (Day 1 to Day 113) in whole blood of subjects in Cohort 3 treated with multiple SC doses (every 4 weeks for 3 doses) of 150 mg of VIB7734 (FIG. 55A), compared to Cohort 3 subjects treated with placebo (FIG. 55B). Additionally, as shown in FIG. 55C, the median of normalized type I IFNGS levels (measured as fold change) over time (Day 1 to Day 85) in whole blood of subjects in cohort 3 treated with multiple SC doses (every 4 weeks for 3 doses) of 150 mg of VIB7734 decreased compared to the median of pDC levels over the same time in whole blood of cohort 3 subjects treated with placebo (FIG. 55C). The median change in type I IFNGS levels in whole blood at Day 85 was −54% in the VIB7734 50 mg group (Cohort 2; FIG. 22C), −83% in the VIB7734 150 mg group (Cohort 3; FIG. 55C), and +8% in the placebo group.

To further evaluate the impact of VIB7734 on circulating type I IFN activity, gene signatures were generated from RNA of whole blood and IFNα protein levels were measured in serum at various timepoints from study participants and healthy donors. The majority of subjects demonstrated high circulating baseline type I IFN activity, with 73% subjects in the combined VIB7734-treated cohorts (i.e., in subjects combined from cohorts 1 (n=3), 2 (n=6), and 3 (n=8)) and 44% of placebo subjects having an IFN-inducible gene expression greater than that in healthy subjects at baseline, with a strong correlation observed between baseline type I IFNGS score and IFNα protein levels (r=0.573; p=0.0203).

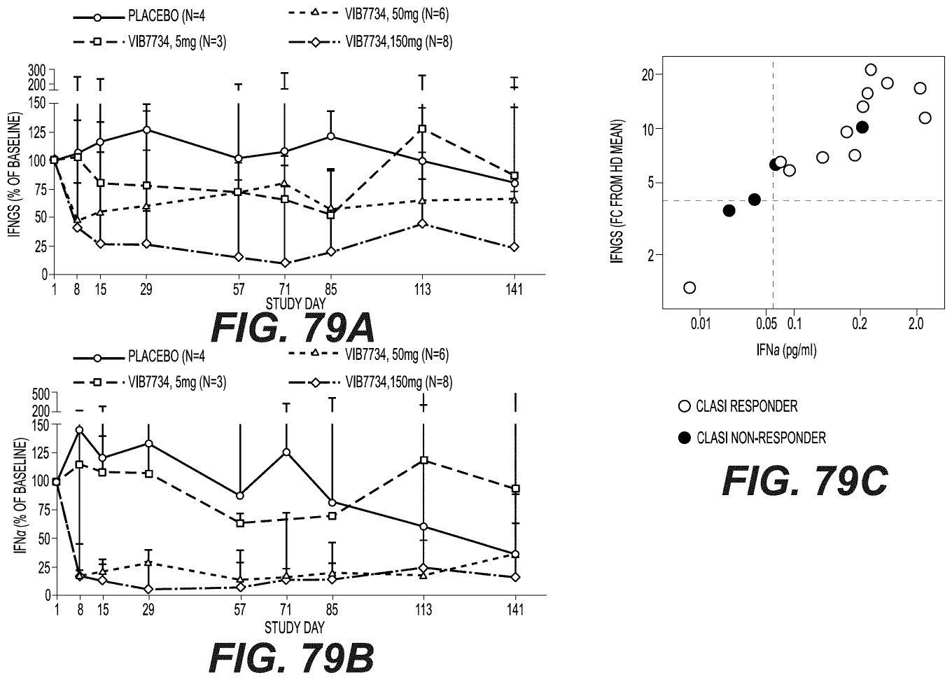

As shown in FIG. 79, among subjects with elevated baseline IFN activity in blood, treatment with VIB7734 led to reductions in both circulating type I IFNGS scores (FIG. 79A) as well as reduction in IFNα protein levels (FIG. 79B), with the most profound and sustained reductions in these measures observed in subjects in the highest dose cohort, i.e., cohort 3 (FIG. 79A-B). At one month post-treatment (day 29), the type I IFNGS score was reduced by 75.07% in subjects in cohort 3 (150 mg VIB7734) compared to an 11.12% increase in the placebo group at that time point. Circulating IFNα protein levels were also dramatically reduced by VIB7734, with the highest dose group (cohort 3) achieving a 95.0% reduction in blood IFNα at day 29 compared to a 33% increase in the placebo group.

The relationship between baseline circulating type I IFN activity and clinical responsiveness to VIB7734 was also assessed in the combined VIB7734-treated cohort. As shown in FIG. 79, high baseline blood type I IFN activity is associated with higher rates of clinical responsiveness to VIB7734. Eleven of 12 subjects demonstrating a clinical response following VIB7734 treatment had high circulating type I IFN activity at baseline, evidenced in both type I IFNGS scores and type I IFN protein (FIG. 79C, black dots). On the other hand, those subjects with lower baseline type I IFN activity were more likely to be CLASI non-responders, with 3 of 4 CLASI non-responders clustering towards lower levels of baseline IFN activity (FIG. 79C, red dots). These data show that high baseline type I IFN activity in the blood is associated with higher rates of responsiveness to pDC depletion therapy.

Clinical development in SLE has been difficult with a number of clinical compounds demonstrating inconsistent responses, likely a reflection of the great heterogeneity of the disease. The present study indicates that baseline type I IFN activity in blood serves as a predictor of clinical responsiveness to pDC depletion, where higher baseline levels of IFNα or type I IFNGS score identifies patients with a higher likelihood of clinical benefit.

### Example 13: Pharmacodynamic Evaluations: Efficacy

The population for the efficacy analyses was subjects with SLE or CLE with an active skin lesion and baseline CLASI score of 8. The CLASI activity (CLASI-A) score ranges from 0 to 70, and can be used to categorize disease activity as mild (0-9), moderate (10-20) or severe (21-70). This population allows testing of the hypothesis that VIB7734 reduces skin manifestations of SLE or CLE. The clinical efficacy endpoint is change in activity score of the CLASI. Both CLASI-A score and CLASI damage score were calculated at Days 1, 15, 29, 43, 57, 85, 113, and 141. Subjects who newly initiated or increased their dose of oral or topical corticosteroids or immunosuppressants in contradiction to the protocol were considered non-responders in the responder analyses. The changes from baseline in CLASI-A was analyzed using mixed-effects model for repeated measures with treatment, baseline type I IFN signature status (low vs. high), visit, and the interaction between visit and treatment as covariates. The proportion of subjects with 4-point reduction in CLASI-A at Day 85 and the proportion of subjects with 50% reduction in CLASI-A at Day 85 was analyzed using a logistic regression with treatment and baseline type I IFN signature status as covariates. Subgroup analyses was conducted by baseline type I IFN signature status (low vs. high) for exploratory purposes.

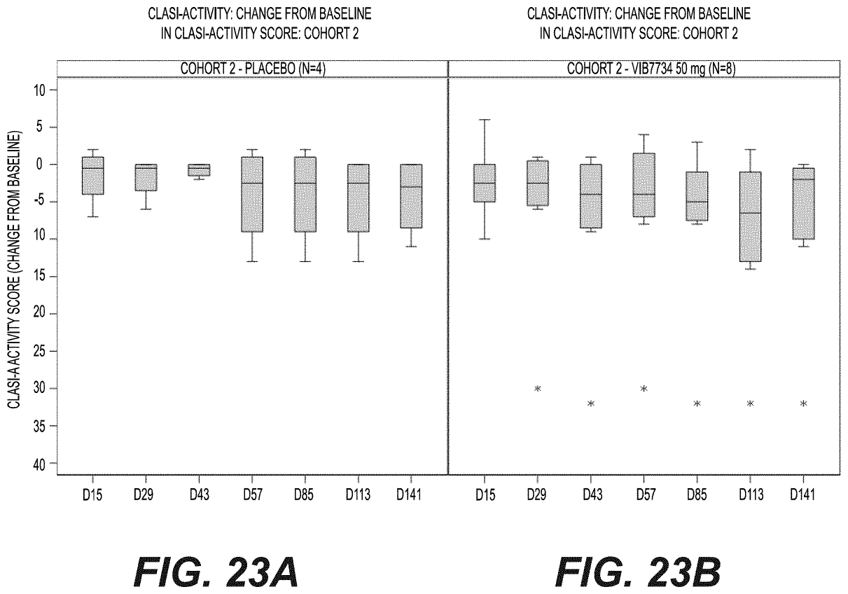

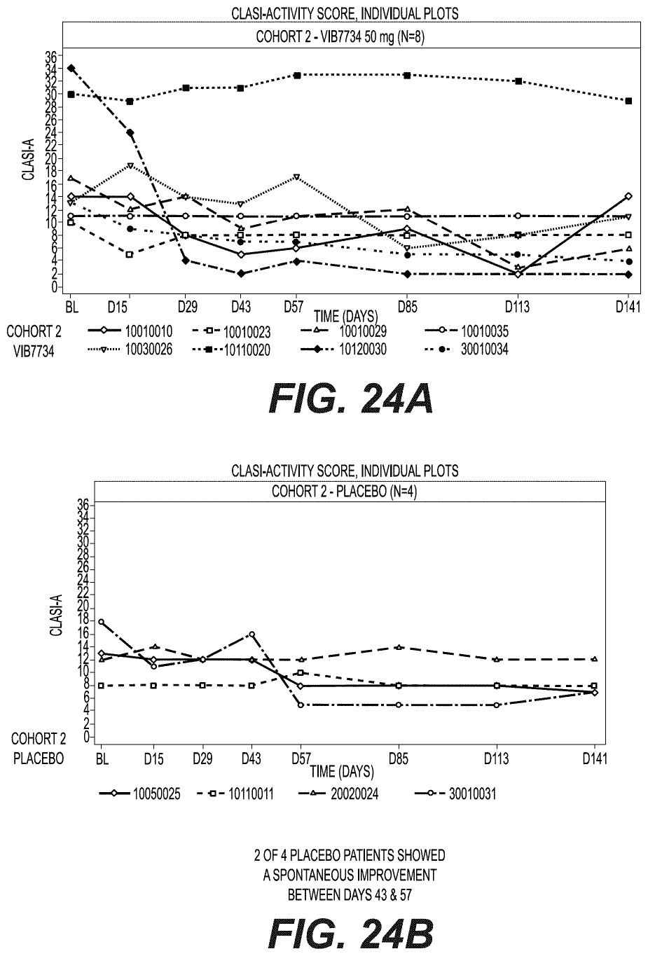

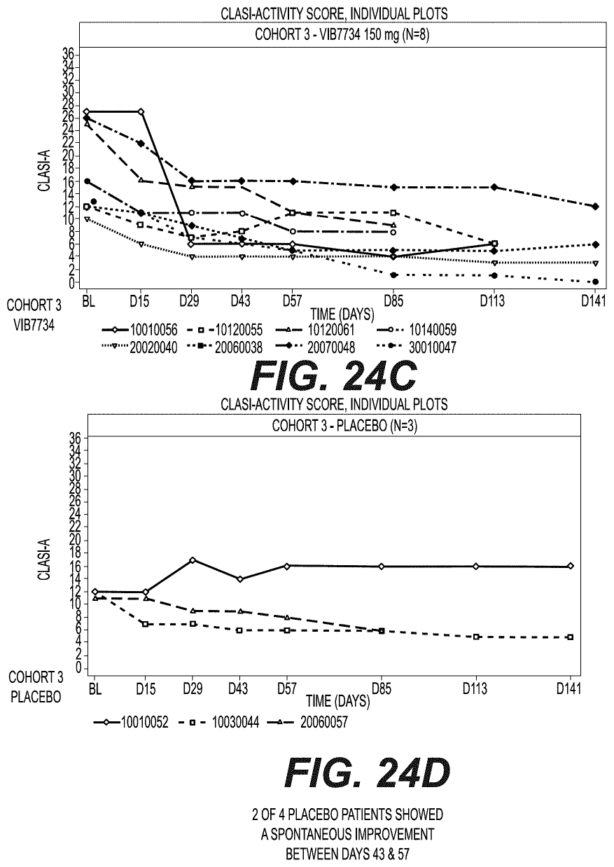

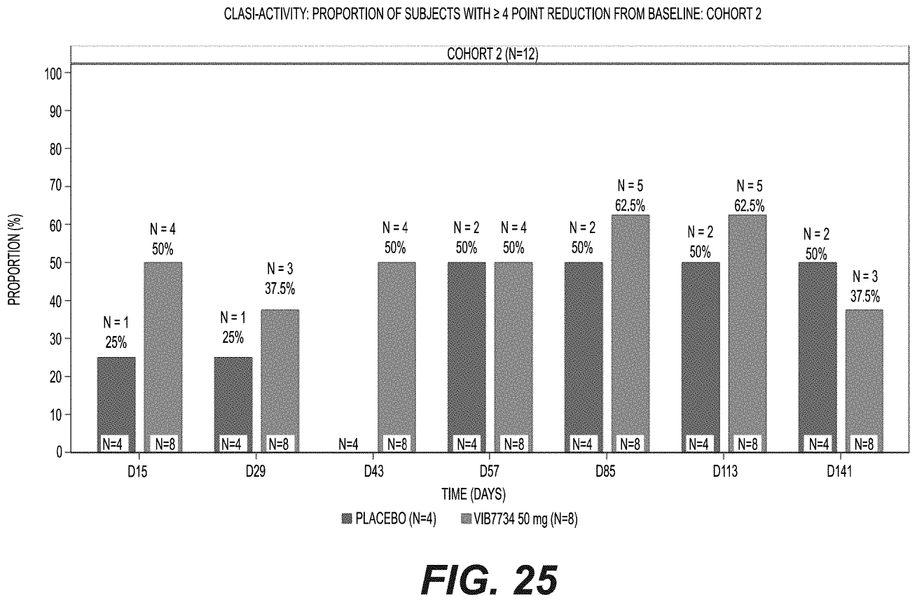

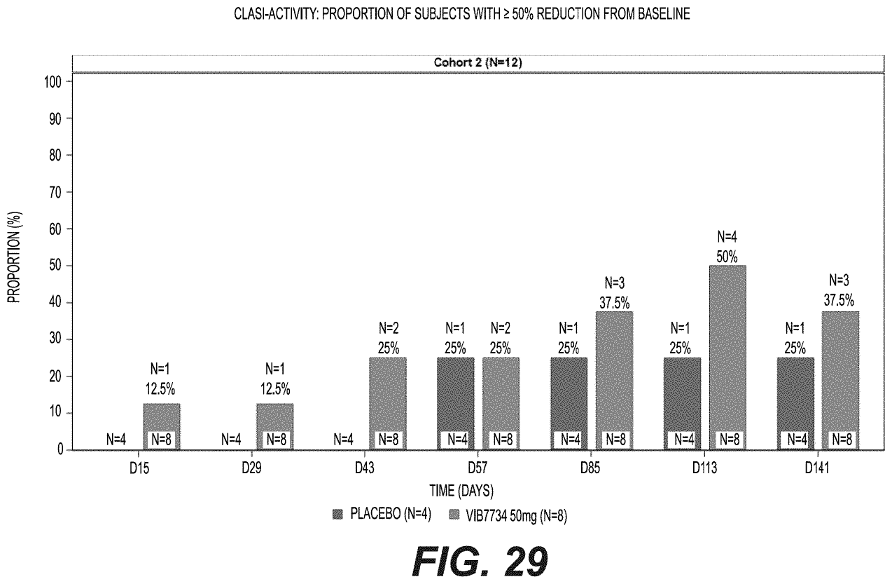

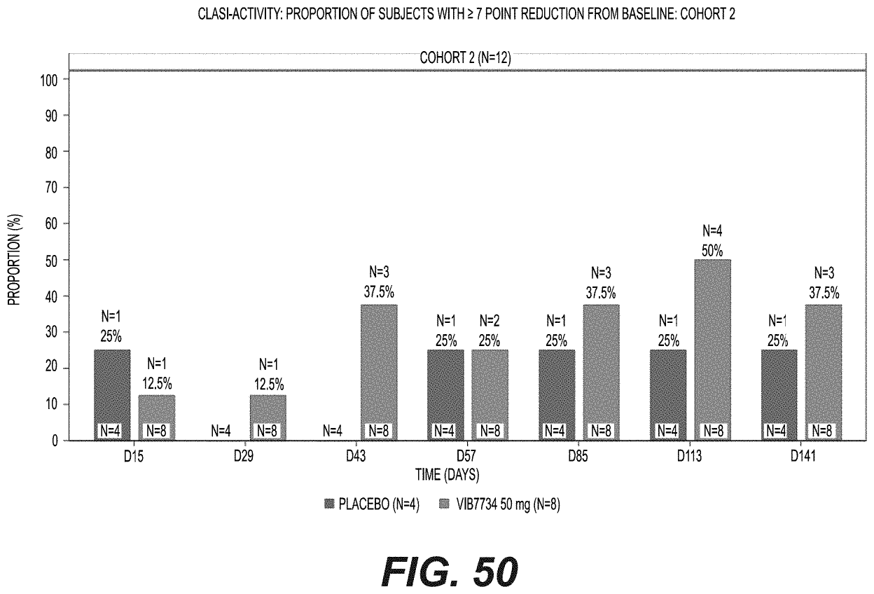

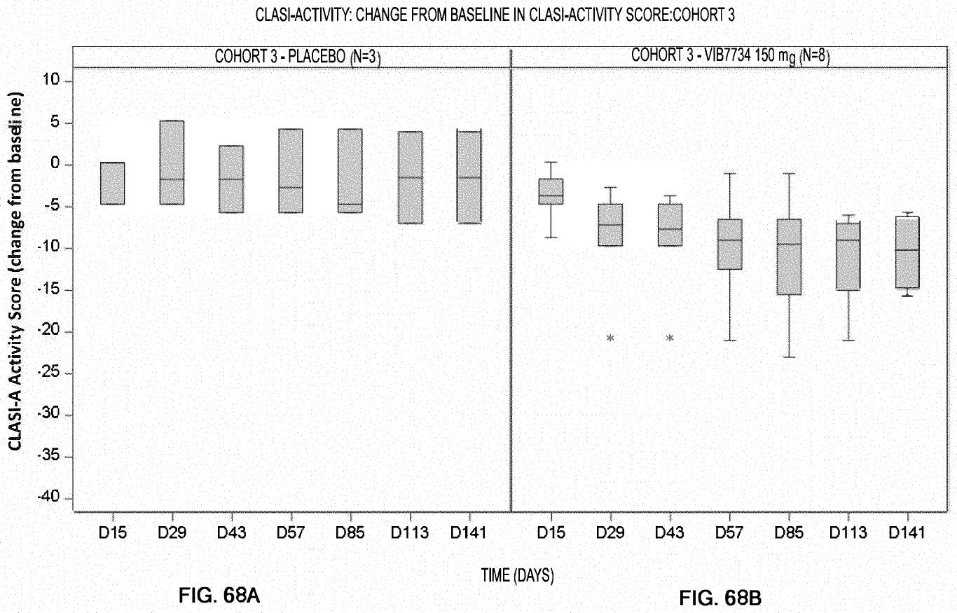

The observed CLASI-A scores along with their changes from baseline are summarized for Cohort 2 subjects (FIGS. 23, 24A, 24B and 33A) and Cohort 3 subjects (FIGS. 68, 24C, 24D, and 33B). The proportion of subjects with at least a 4-point reduction in CLASI-A score from baseline is also summarized for Cohort 2 and Cohort 3 (FIGS. 25-27). Additionally, the proportion of subjects with at least a 7-point reduction in CLASI-A score from baseline is summarized for Cohort 2 and Cohort 3 (FIGS. 50-52). Further, the proportion of Cohort 2 subjects with at least 50% reduction in CLASI-A from baseline is also summarized for Cohort 2 and Cohort 3 (FIGS. 29, 53 and 54).

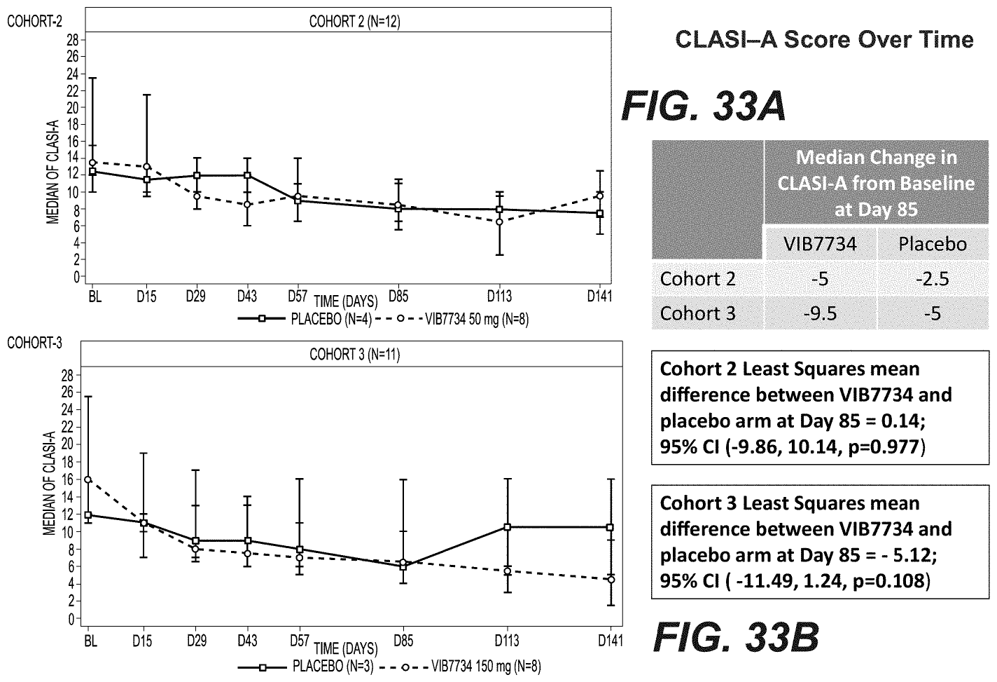

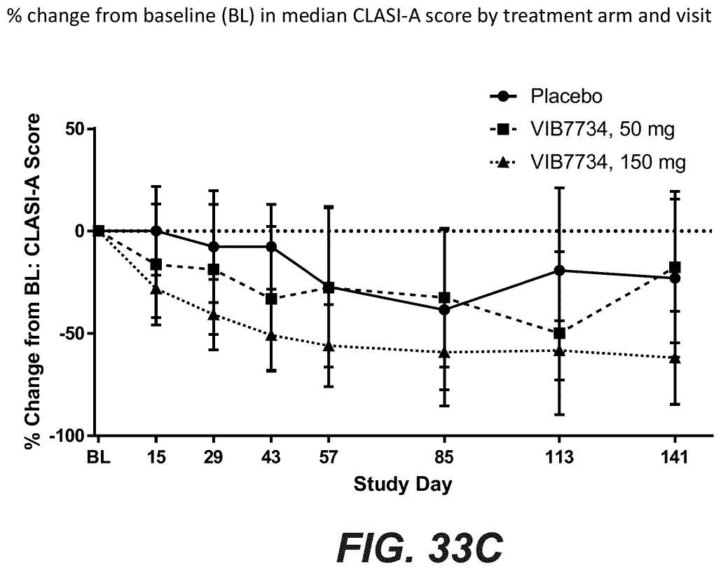

Most subjects in Cohort 2 (FIG. 24A) and all subjects in Cohort 3 (FIG. 24C) showed a decrease in CLASI-A score following multiple SC doses of VIB7734. As shown in FIG. 33, for Cohort 2, at Day 85, the median change in CLASI-A score from baseline was −5.0 for VIB7734-treated subjects compared to −2.5 for placebo-treated subjects (FIG. 33A). The median change in CLASI-A score from baseline at day 85 was unexpectedly higher for Cohort 3 subjects compared to that for Cohort 2 subjects. As shown in FIG. 33, for Cohort 3, at Day 85, the median change in CLASI-A score from baseline was −9.5 for VIB7734-treated subjects compared to −5.0 for placebo-treated subjects (FIG. 33B). For Cohort 2 subjects, Least Squares mean difference between the VIB7734 and the placebo arm at Day 85 was 0.14; 95% Cl (−9.86, 10.14, p=0.977). In addition, for Cohort 3, Least Square mean difference between the VIB7734 arm and the placebo arm at Day 85 was −5.24 (95%, Cl −11.8, 1.3, p=0.11) (FIG. 33B). FIG. 33C shows the percentage change from baseline (BL) in median CLASI-A score by treatment arm and visit for subjects in Cohort 2 and Cohort 3.

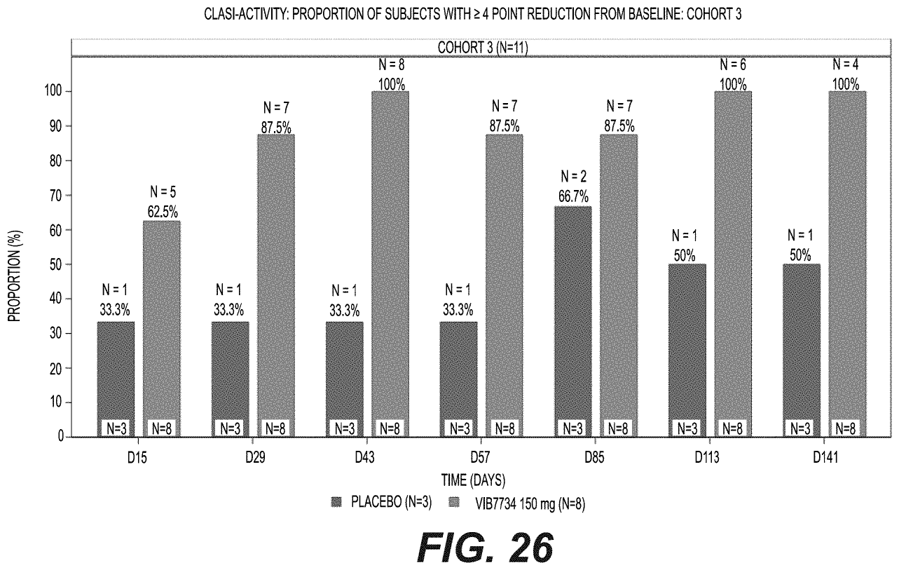

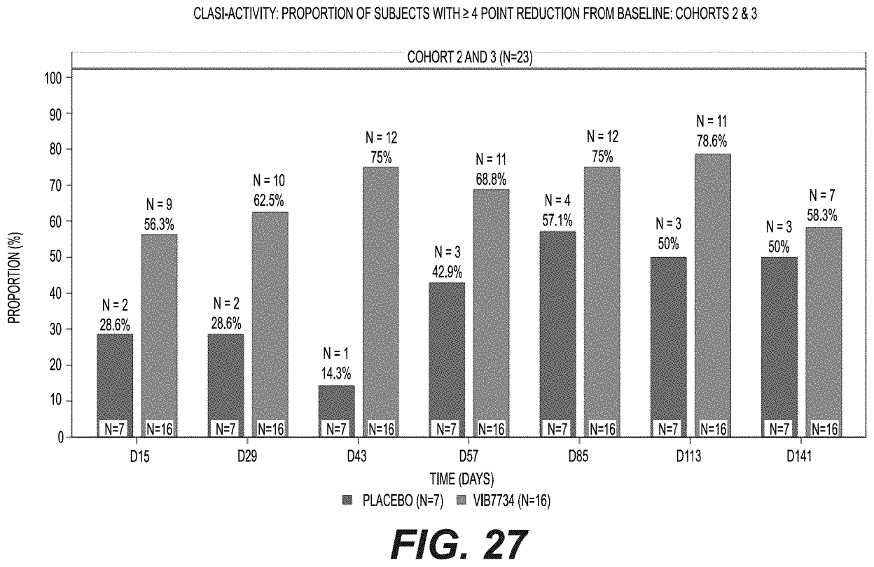

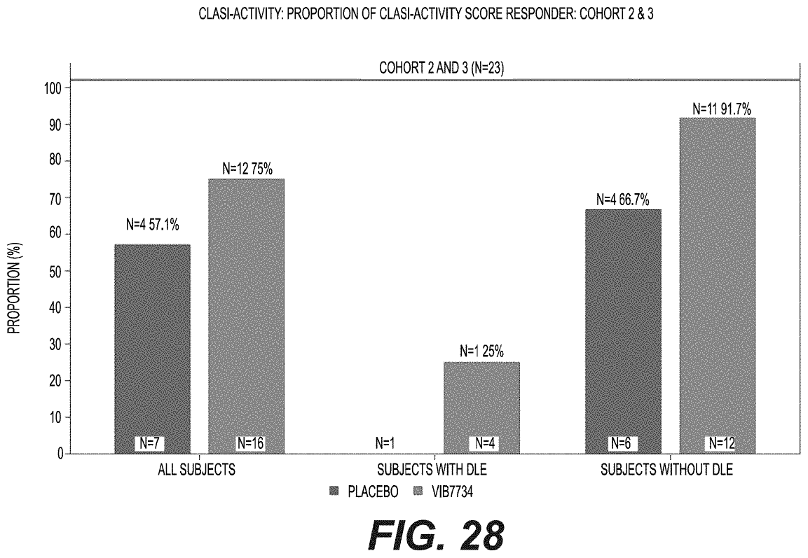

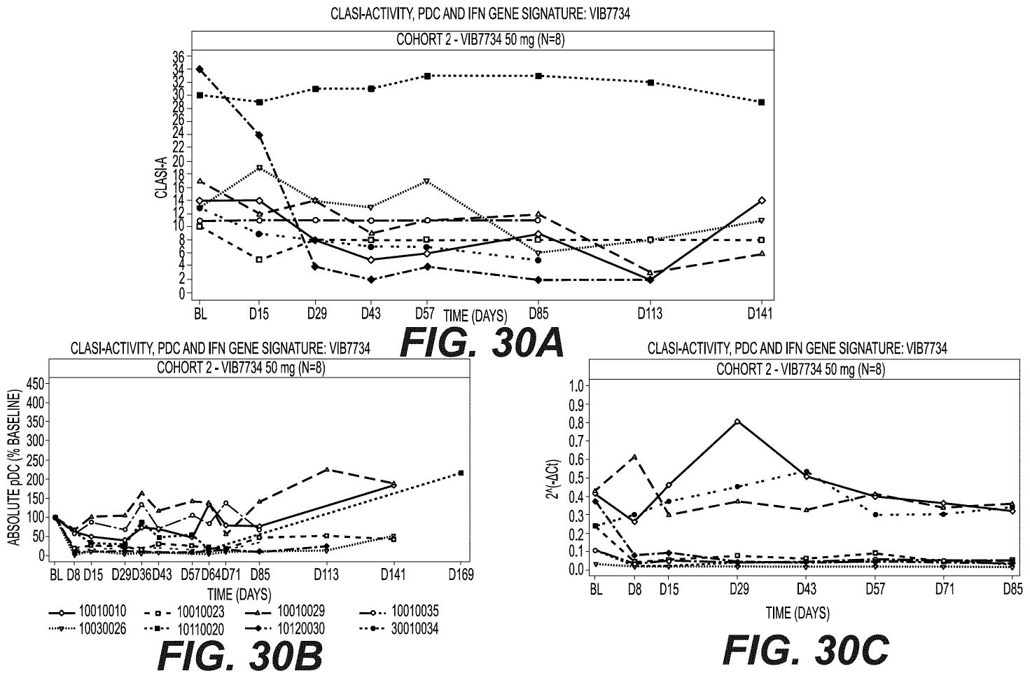

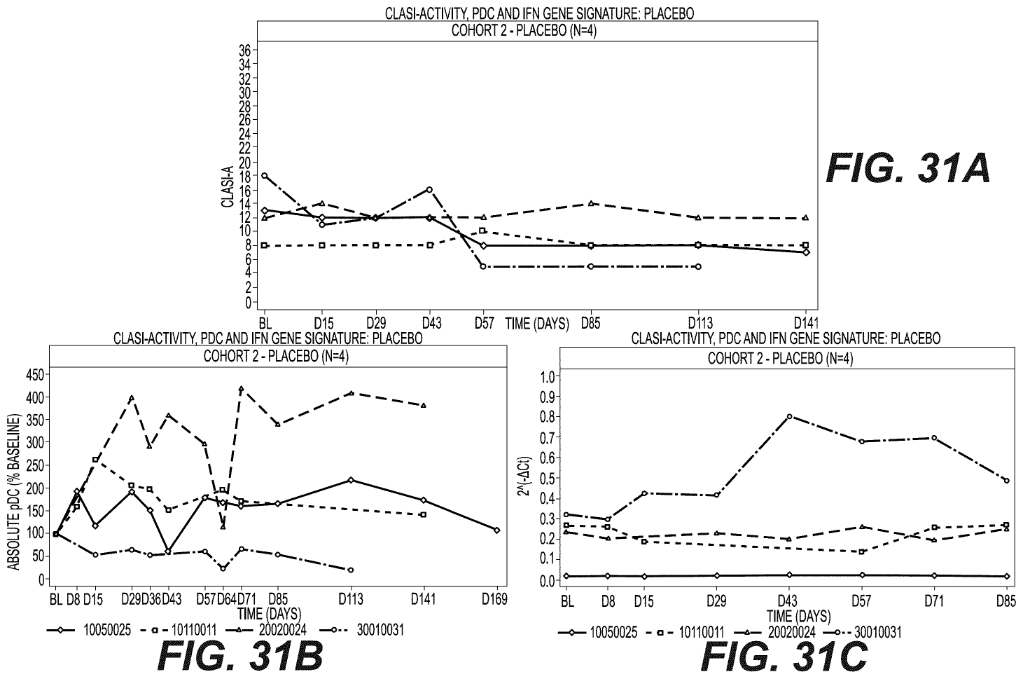

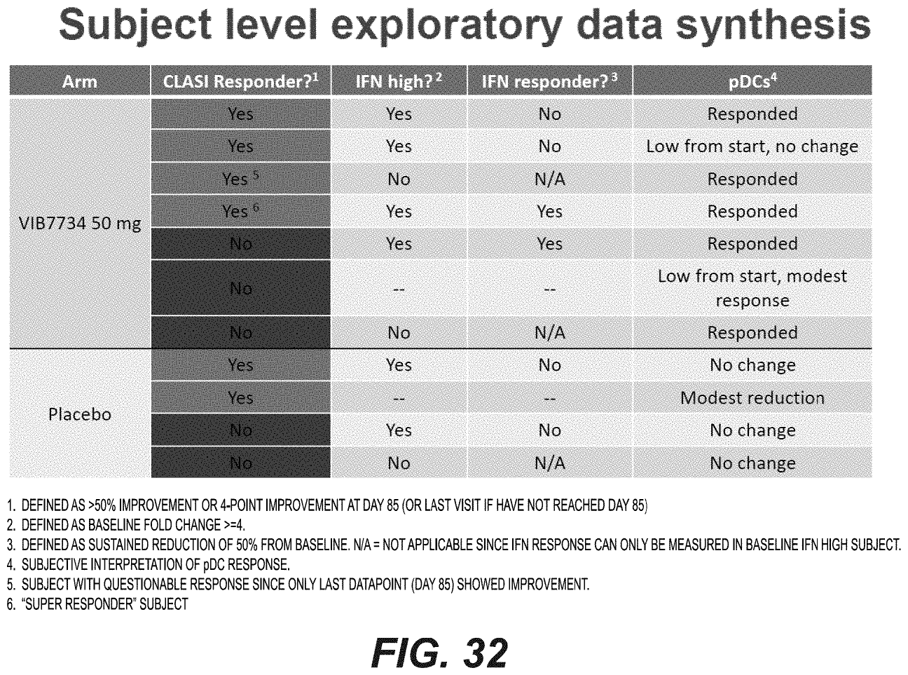

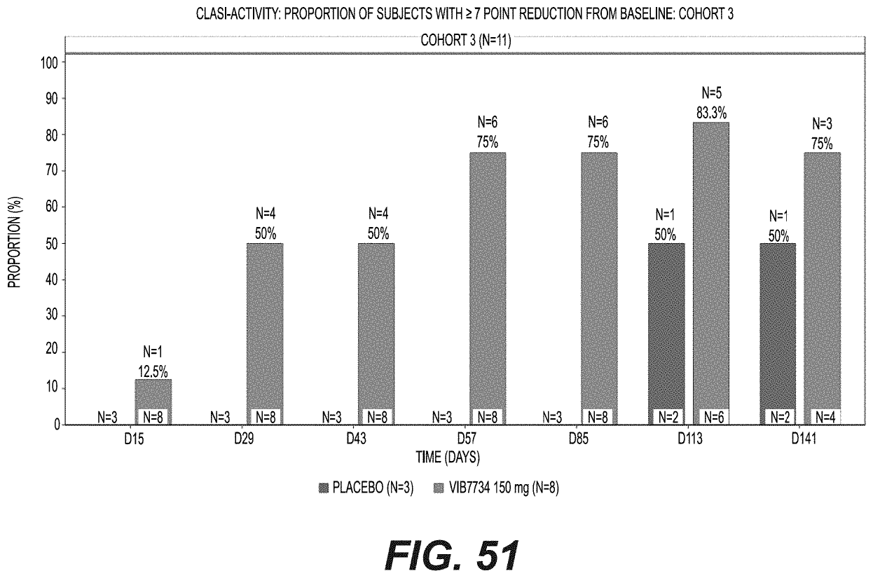

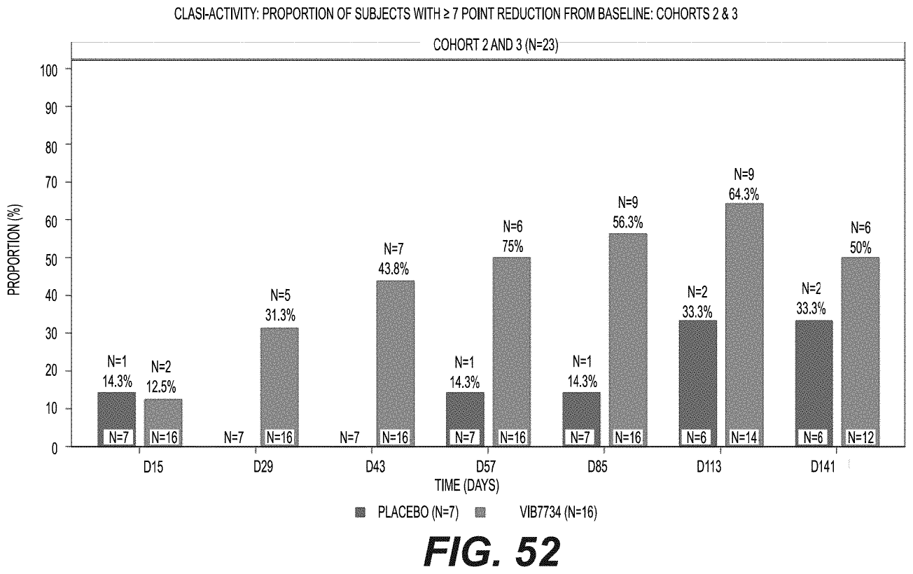

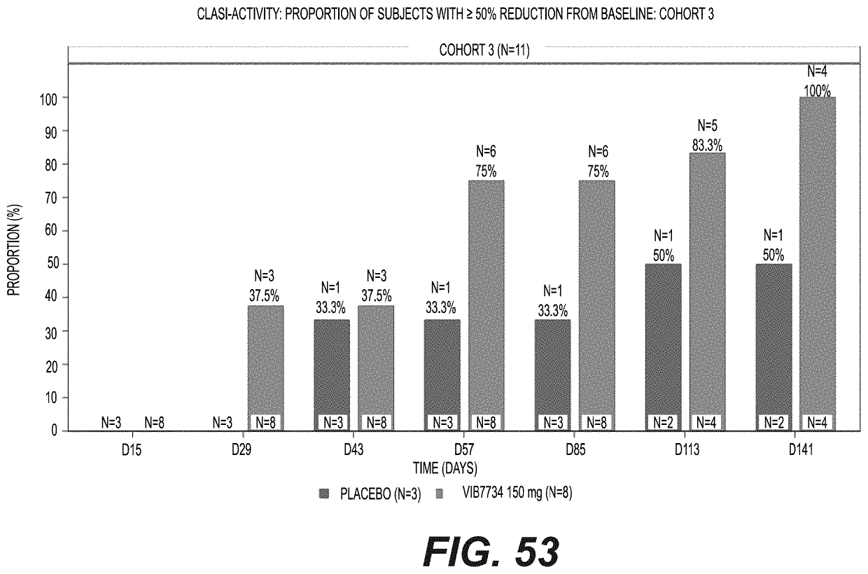

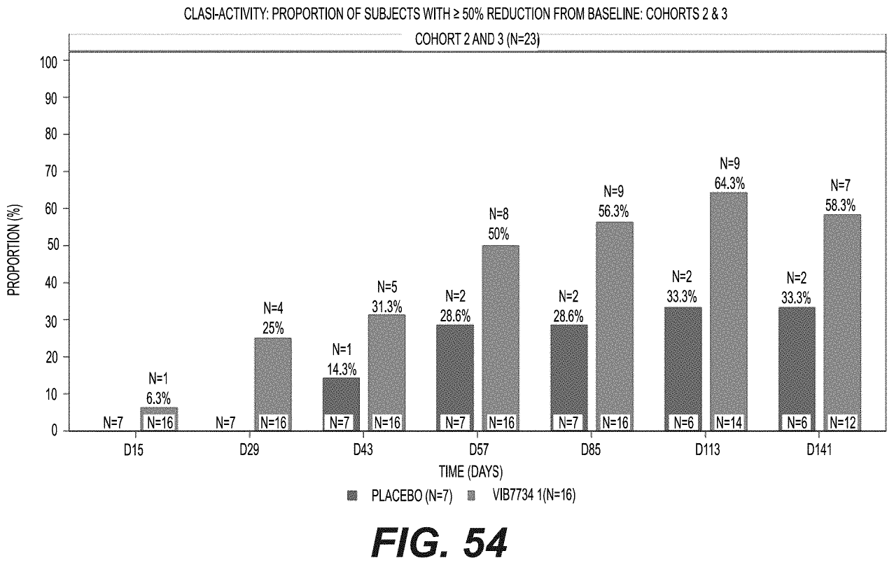

Further, at days 15, 29, 85 and 113, a higher proportion of VIB7734-treated subjects in Cohort 2 showed an at least 4-point reduction in CLASI-A score from baseline compared to placebo-treated Cohort 2 subjects (FIG. 25). At days 15, 29, 43, 57, 85 and 113, a higher proportion of VIB7734-treated subjects in Cohort 3 showed an at least 4-point reduction in CLASI-A score from baseline compared to placebo-treated Cohort 3 subjects (FIG. 26). The same trend was observed when data from Cohort 2 and 3 subjects were combined (FIG. 27). Additionally, at days 15, 29, 43, 57, 85, 113 and 141, a higher proportion of VIB7734-treated subjects in Cohort 2 (FIG. 50) and Cohort 3 (FIG. 51) showed an at least 7-point reduction in CLASI-A score from baseline compared to placebo-treated Cohort 2 subjects (FIG. 50) and Cohort 3 subjects (FIG. 51), respectively. The same trend was observed when data from Cohort 2 and 3 subjects were combined (FIG. 52). Further, when data from Cohort 2 and Cohort 3 were combined, a greater proportion of CLASI-A score responders were observed in VIB7734-treated subjects (75%) compared to subjects treated with a placebo (57.1%) (FIG. 28). When the subset of subjects in Cohort 2 and Cohort 3 without discoid lupus erythematosus (DLE) were considered, the proportion of CLASI-A score responders in VIB7734-treated subjects was further increased (91.7%) compared to placebo-treated subjects (66.7%) (FIG. 28). Additionally, at days 15, 29, 43, 57, 85, 113 and 141, an at least 50% reduction in CLASI-A score from baseline was observed in a higher proportion of VIB7734-treated Cohort 2 subjects compared to Cohort 2 subjects treated with a placebo (FIG. 29). Similarly, at days 29, 43, 57, 85, 113 and 141, an at least 50% reduction in CLASI-A score from baseline was observed in a higher proportion of VIB7734-treated Cohort 3 subjects compared to Cohort 3 subjects treated with a placebo (FIG. 53). The same trend was observed when data from Cohort 2 and 3 subjects were combined (FIG. 54). As shown in FIG. 54, at Day 85, a ≥50% improvement in CLASI-A was observed in 9 of 16 (˜56%) VIB7734-treated subjects and 2 of 7 (˜29%) placebo-treated subjects. FIGS. 30 and 31 summarize the changes observed in CLASI-A score (FIG. 30A), absolute pDC blood levels (FIG. 30B) and blood type I IFNGS levels (measured as absolute score) (FIG. 30C) over time for subjects in Cohort 2 treated with VIB7734 compared to changes in CLASI-A score (FIG. 31A), absolute blood pDC levels (FIG. 31B) and blood type I IFNGS levels (measured as absolute score) (FIG. 31C) over time for Cohort 2 subjects treated with a placebo. CLASI-A score and absolute pDC blood levels were decreased and the neutralization ratios of blood type I IFNGS levels were increased in in the VIB7734-treated group compared to the placebo-treated group.

### Example 14: Pharmacodynamic Evaluations: Skin pDC and IFN-1 Levels

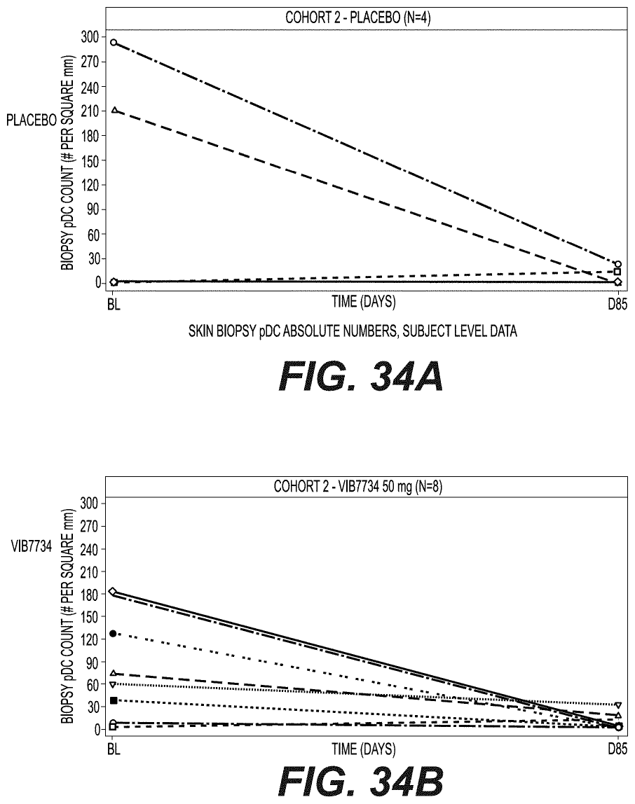

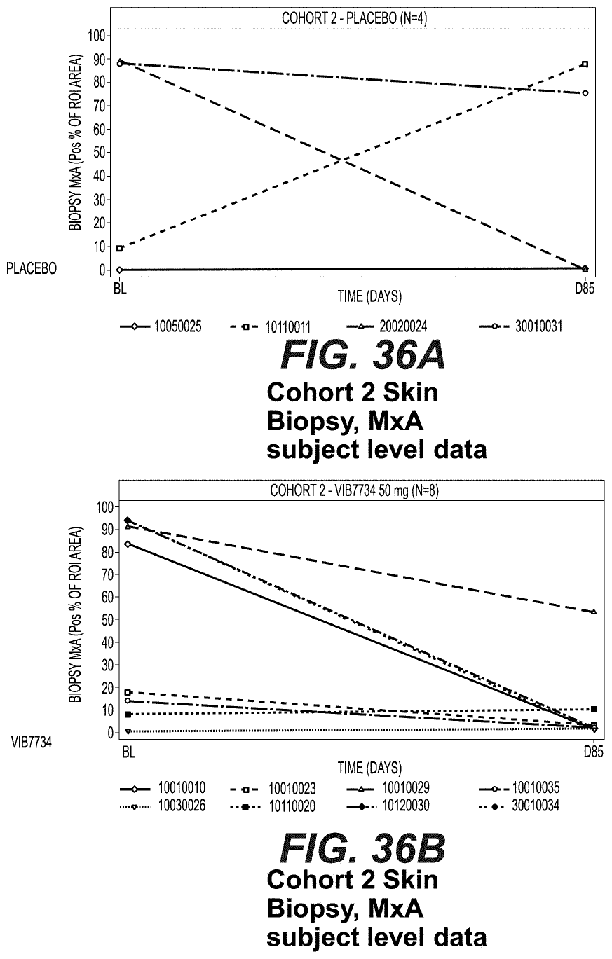

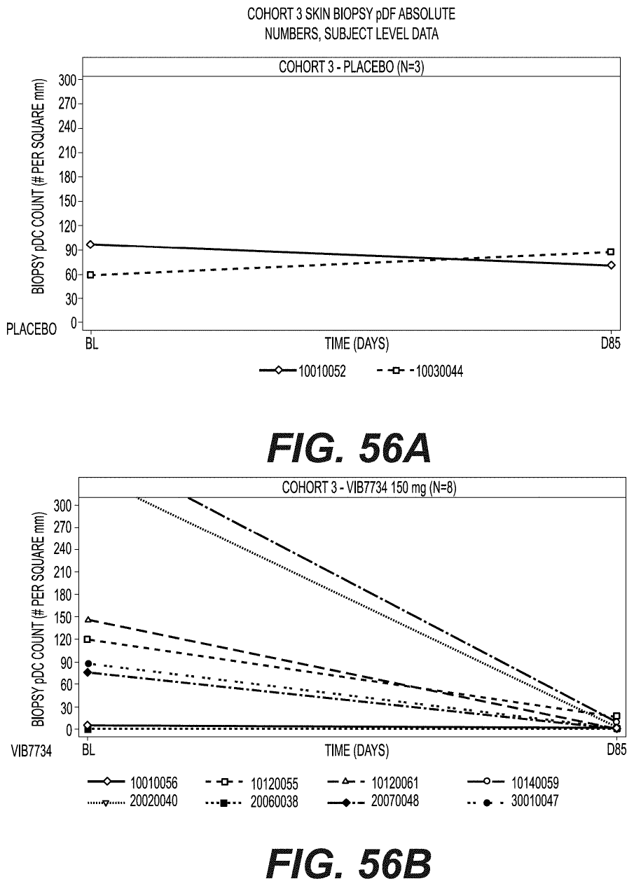

Skin biopsy was conducted for subjects in Cohorts 2 and 3, and the effect of VIB7734 on pDC levels and Type 1 interferon (IFN-1) activity (assayed by measuring Myxovirus protein A (MxA) levels) from the skin biopsy was evaluated. The results are presented in FIGS. 34-37 (Cohort 2) and FIGS. 56-59 (Cohort 3).

Each skin biopsy requires one 4 mm punch biopsy. The anatomic site selected for biopsy was an area of active inflammation as indicated by erythema or scale. The punch biopsy site was closed with a single suture. The baseline skin biopsy was performed prior to dosing on Day 1 or during the screening period. However, the baseline skin biopsy was not performed until other screening procedures had confirmed that the subject is eligible for the study. A repeat biopsy was performed on Day 85 (±14 days) or at the Early Discontinuation Visit if the subject discontinued the study prior to the Day 85 visit. The Day 85 biopsy was taken from the same anatomic site adjacent to the baseline biopsy site, avoiding the scar tissue from the previous punch biopsy. The effect of VIB7734 on pDCs in skin lesions was measured by evaluating the number of pDCs/mm2 in skin biopsy samples before and after drug administration. pDCs were identified using an anti-ILT7 clone. The rationale for measuring the change in pDC density in affected skin was to confirm that VIB7734 depletes pDCs in a target tissue in addition to blood, and to determine if there is a difference between the dose necessary to achieve a target level of pDC depletion in the blood as compared to the skin. This will assist with dose selection for subsequent clinical trials. The density of all inflammatory cells were also measured. This demonstrated whether reducing pDC levels leads to downstream effects on the density of other inflammatory cells in the skin. The observed pDC levels and level as a percent of baseline were summarized.

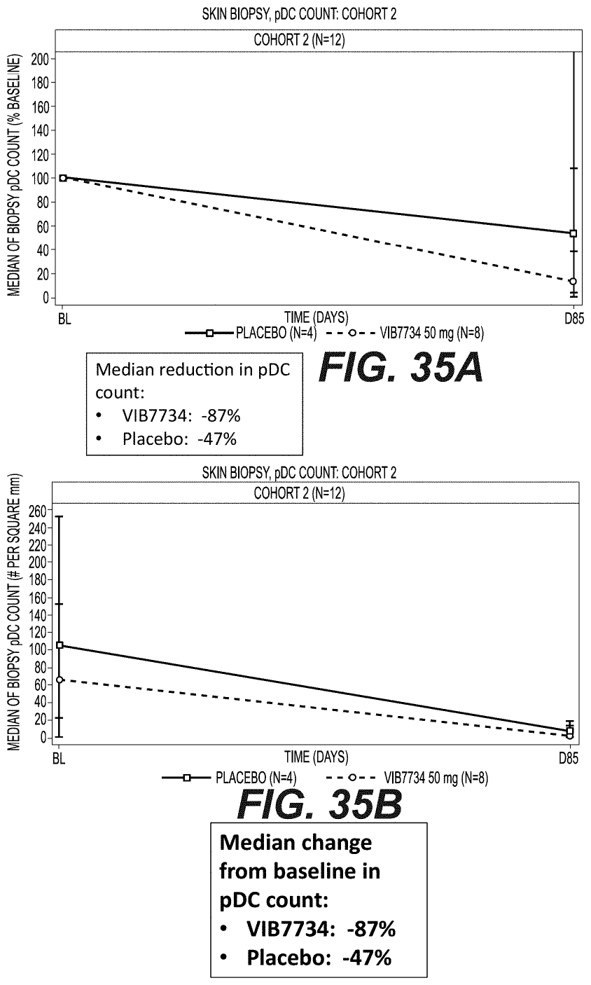

FIG. 34 shows absolute biopsy pDC count (measured as number of cells per square mm) over time in skin biopsies of subjects in Cohort 2 following multiple subcutaneous doses (every 4 weeks for 3 doses) of 50 mg of VIB7734 (FIG. 34B) or a placebo (FIG. 34A). As shown in FIG. 35, for subjects in Cohort 2, the median reduction of change in skin biopsy pDC count on Day 85 (measured as a percent of Day 1 baseline (FIG. 35A) as well as number of cells per square mm (FIG. 35B)) was 87% for VIB7734-treated Cohort 2 subjects compared to 47% for Cohort 2 subjects treated with a placebo.

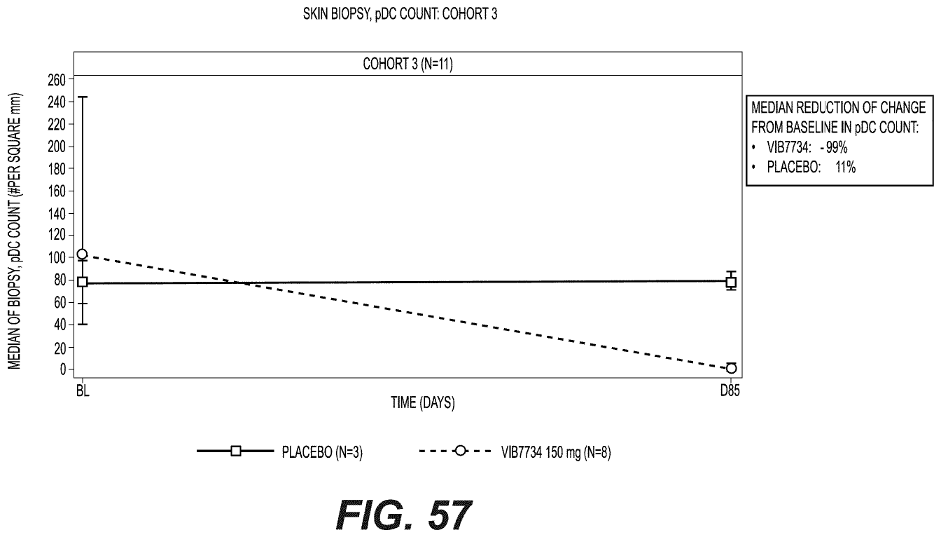

FIG. 56 shows absolute biopsy pDC count (measured as number of cells per square mm) over time in skin biopsies of subjects in Cohort 3 following multiple subcutaneous doses (every 4 weeks for 3 doses) of 150 mg of VIB7734 (FIG. 56B) or a placebo (FIG. 56A). As shown in FIG. 57, the median of skin biopsy pDC count on Day 85 (measured as a percent of Day 1 baseline) was reduced by 99% for VIB7734-treated Cohort 3 subjects compared to an increase of 11% from baseline for Cohort 3 subjects treated with placebo. Combining the placebo data from Cohorts 2 and 3, the median change in of skin biopsy pDC density on Day 85 (measured as a percent of Day 1 baseline) was −87% for the 50 mg group (Cohort 2), −99% for the 150 mg group (Cohort 3), and −14% for the placebo group.

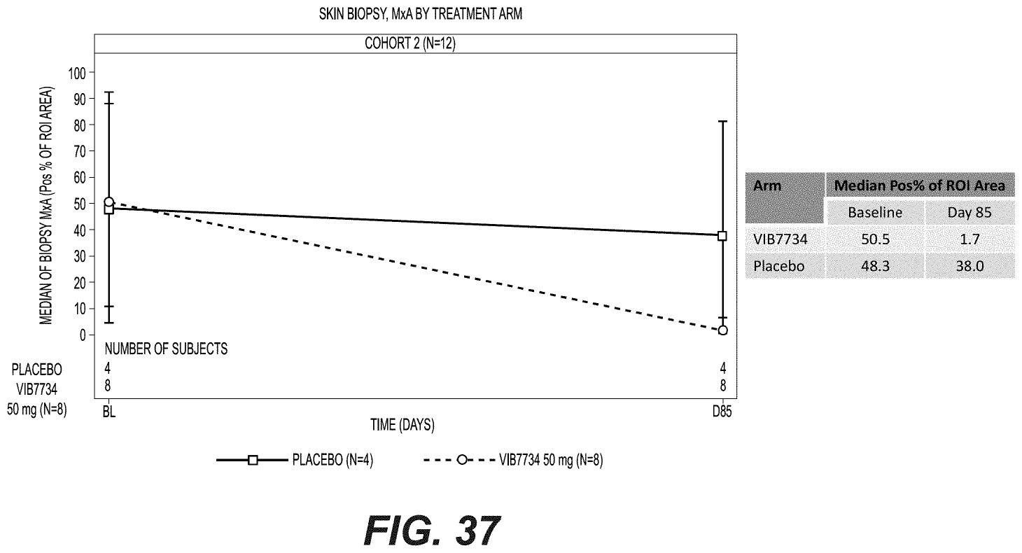

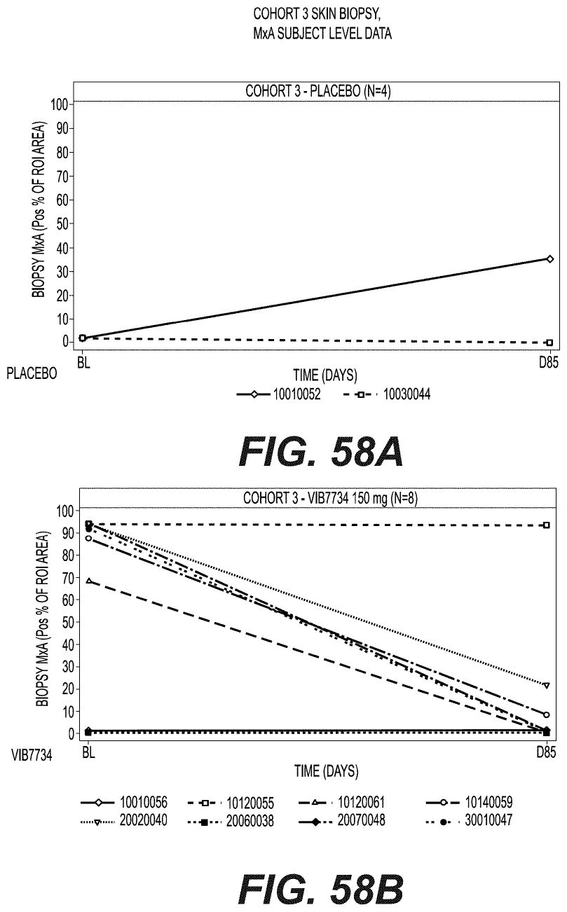

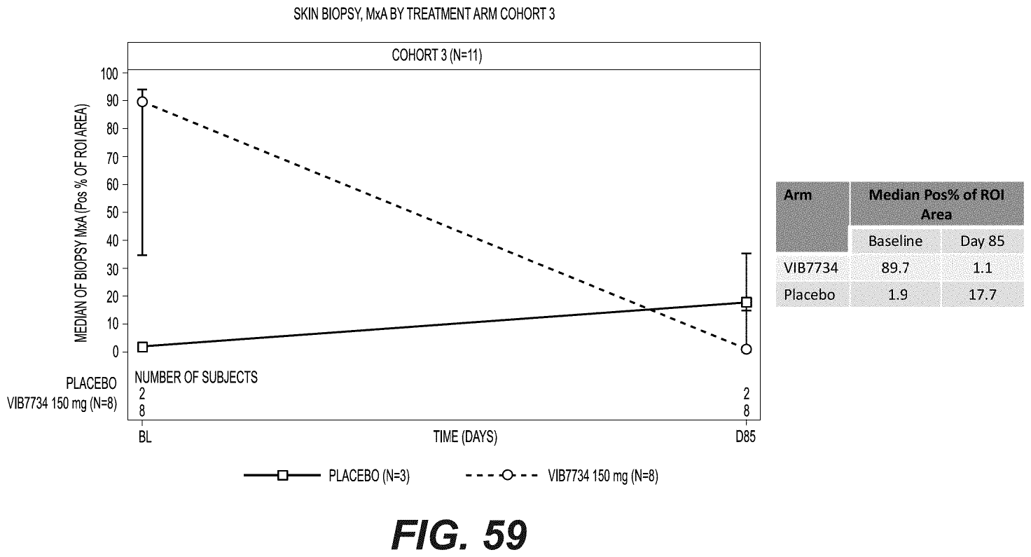

Type 1 interferon (IFN-1) activity is upregulated in skin lesions in patients with CLE. The effect of VIB7734 on IFN-1 activity in skin lesions was also determined by assaying levels of Myxovirus protein A (MxA), an interferon regulated protein in skin biopsies from subjects in Cohort 2 (FIG. 37) and Cohort 3 (FIG. 58). As shown in FIG. 37, for VIB7734-treated subjects in Cohort 2, the median of biopsy MxA (measured as percent area positive for MxA; Pos % of ROI area) was reduced from a baseline value of 50.5% on Day 1 to 1.7% on Day 85. In contrast, for placebo-treated Cohort 2 subjects, the median of biopsy MxA was only reduced from a baseline value of 48.3 on Day 1 to 38.0 on Day 85. As further shown in FIG. 59, for VIB7734-treated subjects in Cohort 3, the median of skin biopsy MxA (measured as percent area positive for MxA; Pos % of ROI area) was reduced from a baseline value of 89.7% to 1.1% on Day 85. In contrast, for placebo-treated Cohort 3 subjects, the median of skin biopsy MxA actually increased from a baseline value of 1.9% to 17.7% on Day 85. Combining the placebo data from Cohorts 2 and 3, the median area of MxA staining decreased with VIB7734 treatment from day 1(baseline) to day 85: 50 mg group (Cohort 2): 50% to 1.7% affected area; 150 mg group (Cohort 3): 90% to 1.1% affected area; placebo group: 5.4% to 18% affected area.

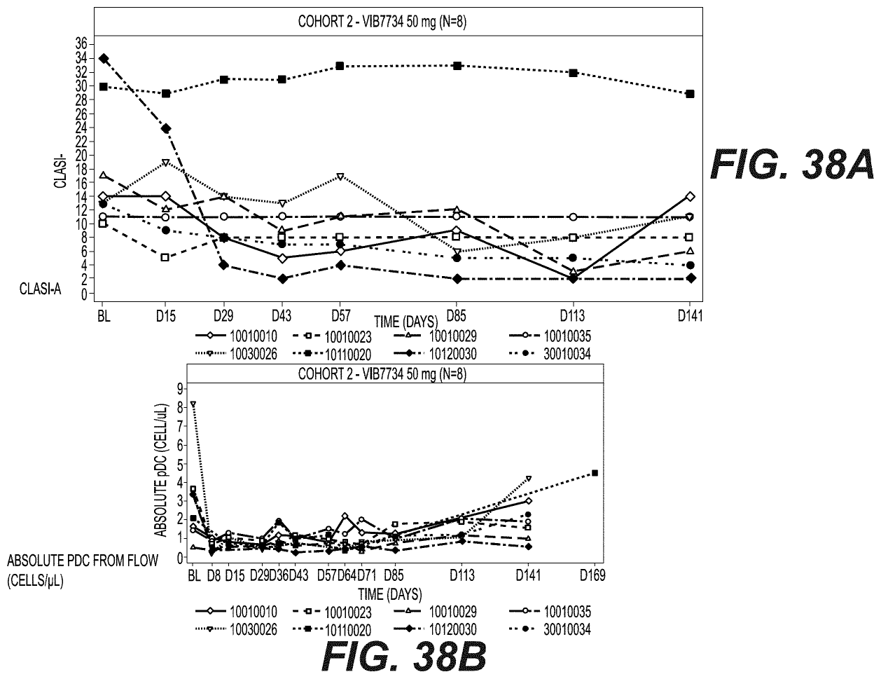

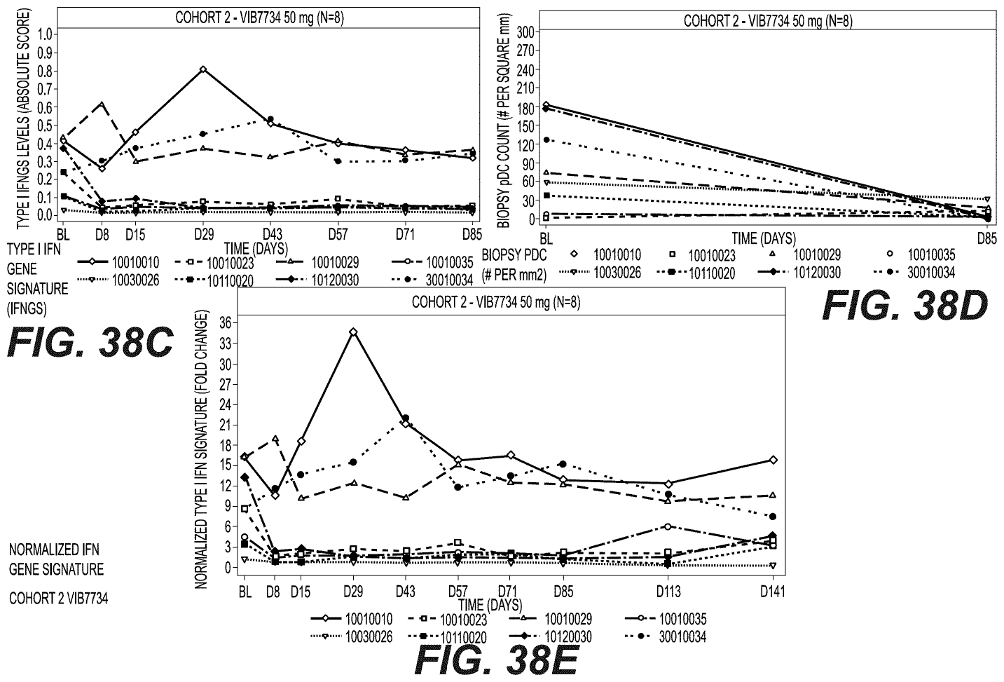

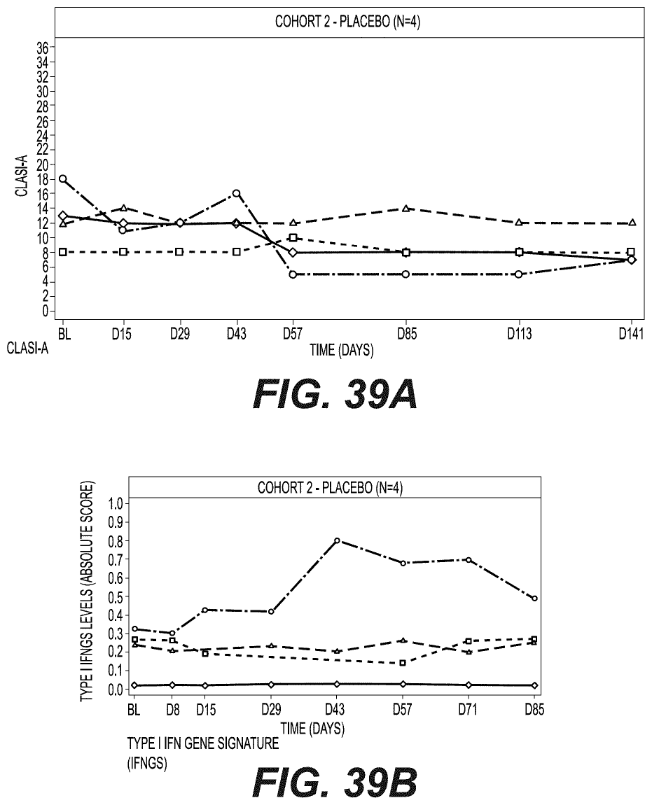

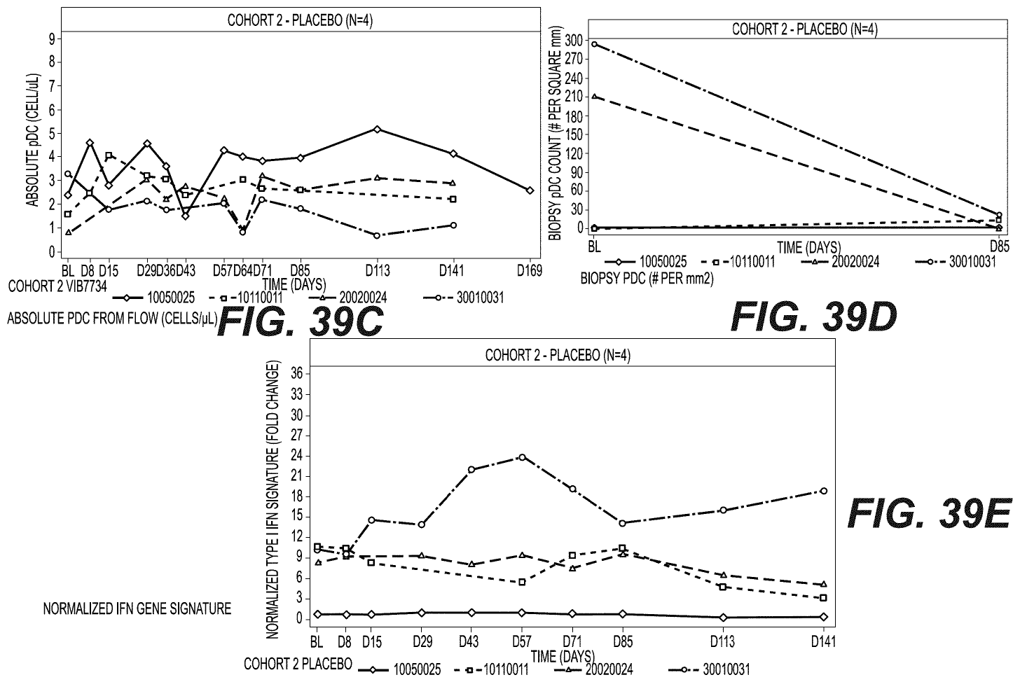

FIGS. 38 and 39 summarize the changes observed in CLASI-A score (FIG. 38A), absolute pDC blood levels (FIG. 38B), blood type I IFNGS levels (measured as absolute score) (FIG. 38C), skin biopsy pDC counts (FIG. 38D), and blood normalized type I IFNGS levels (measured as fold change) (FIG. 38E) over time for VIB7734-treated Cohort 2 subjects compared to changes in CLASI-A score (FIG. 39A), absolute blood pDC levels (FIG. 39B), blood type I IFNGS levels (measured as absolute score) (FIG. 39C), skin biopsy pDC counts (FIG. 39D), and blood normalized type I IFNGS levels (measured as fold change) (FIG. 39E) over time for placebo-treated Cohort 2 subjects. CLASI-A scores, absolute pDC blood levels, blood type I IFNGS levels, and skin biopsy pDC counts were all decreased in the VIB7734-treated Cohort 2 group compared to the placebo-treated Cohort 2 group.

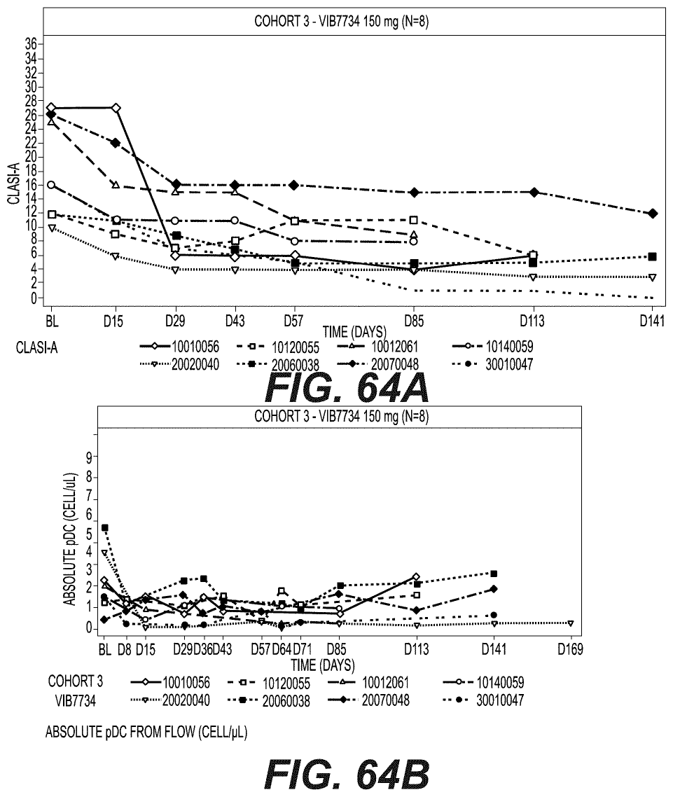

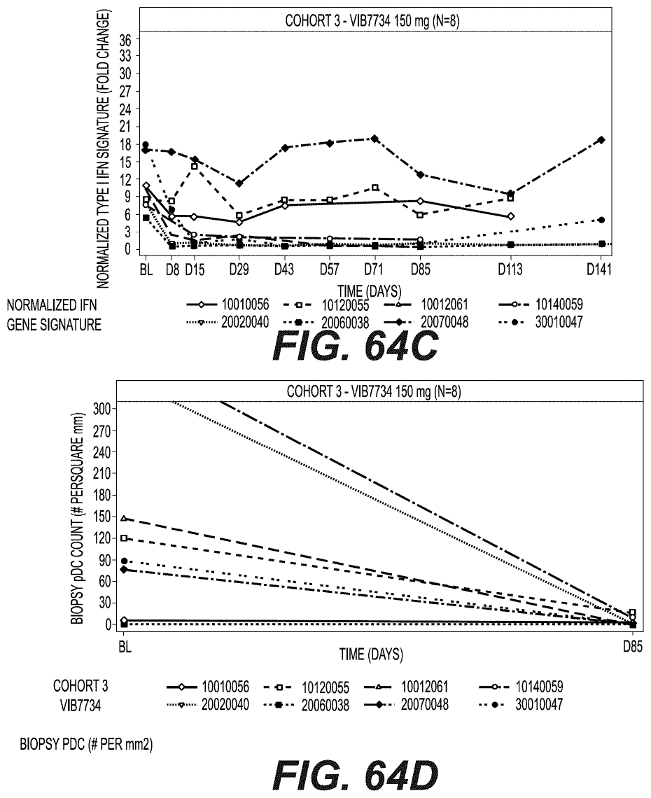

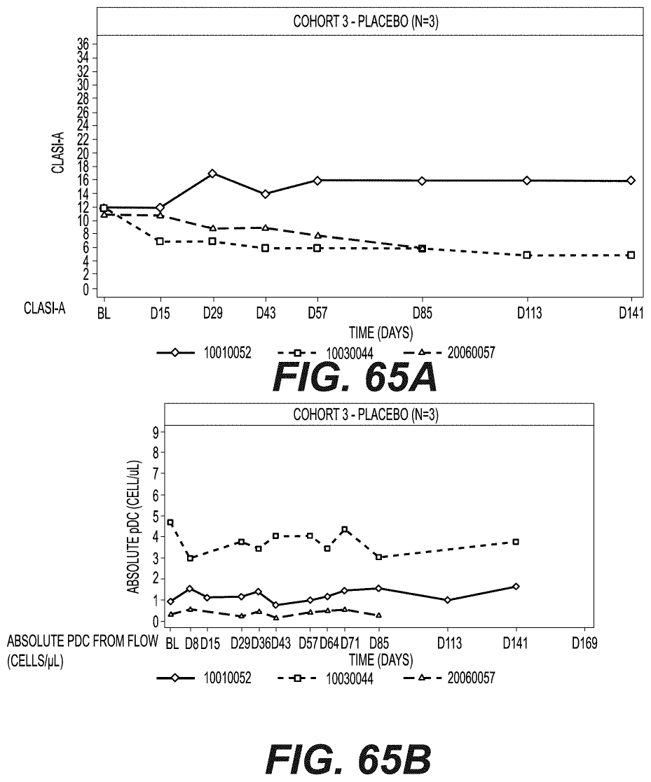

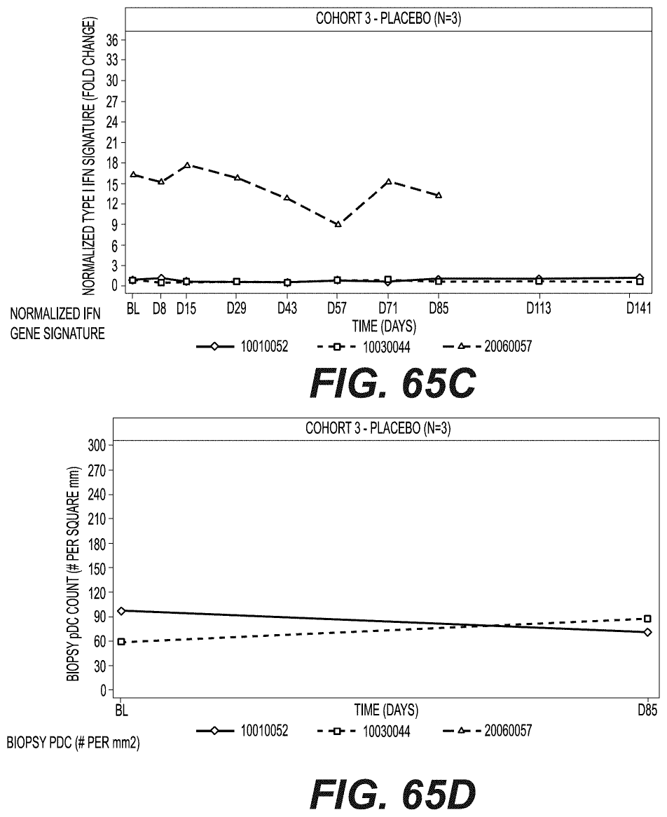

FIGS. 64 and 65 summarize the changes observed in CLASI-A score (FIG. 64A), absolute pDC blood levels (measured as cells/μL) (FIG. 64B), blood normalized type I IFNGS levels (measured as fold change) (FIG. 64C) and skin biopsy pDC count (measured as number of cells per square mm) (FIG. 64D) over time for VIB7734-treated Cohort 3 subjects compared to changes in CLASI-A score (FIG. 65A), absolute pDC blood levels (measured as cells/μL) (FIG. 65B), blood normalized type I IFNGS levels (measured as fold change) (FIG. 65C) and skin biopsy pDC count (measured as number of cells per square mm) (FIG. 65D) over time for placebo-treated Cohort 3 subjects. CLASI-A scores, absolute pDC blood levels, blood type I IFNGS levels, and skin biopsy pDC counts were all decreased in the VIB7734-treated Cohort 3 group compared to the placebo-treated Cohort 3 group.

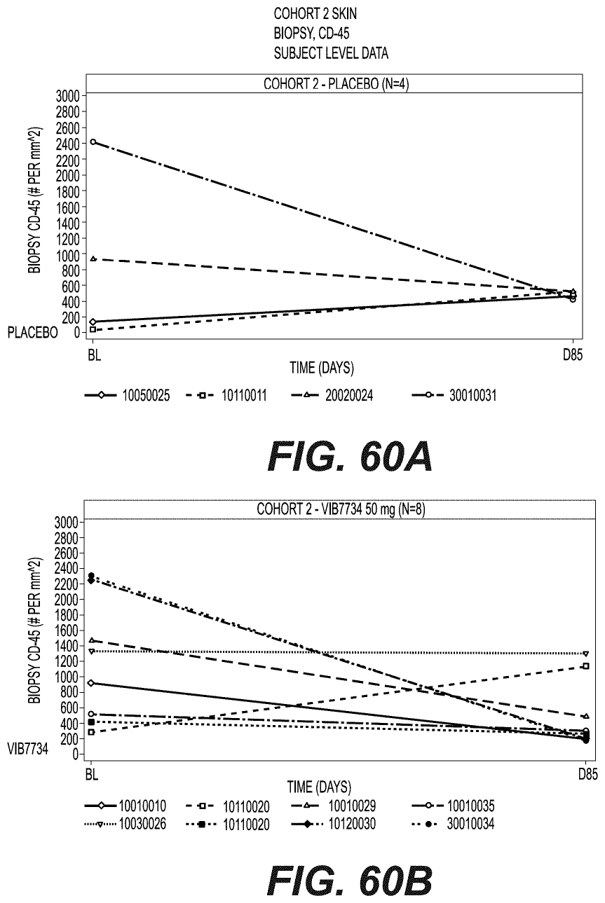

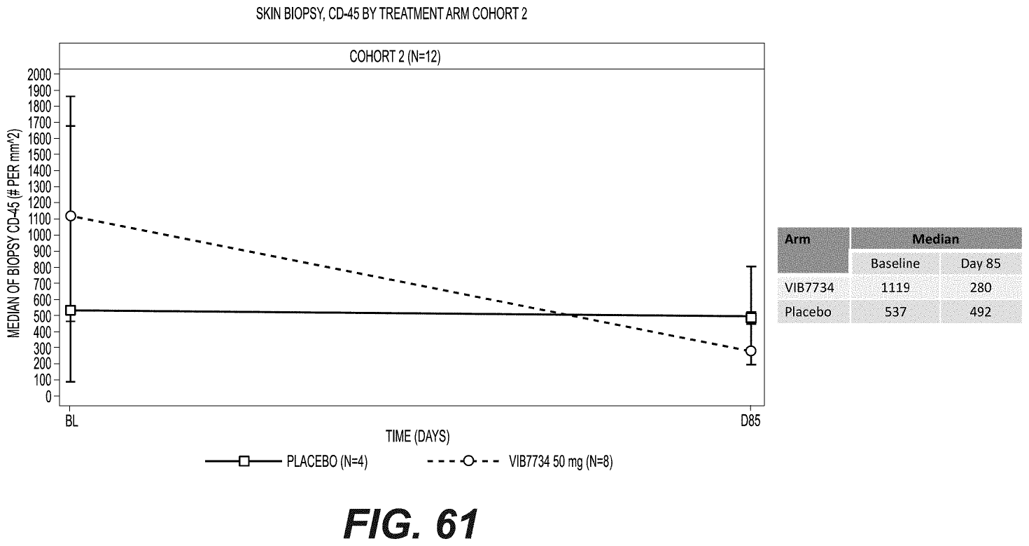

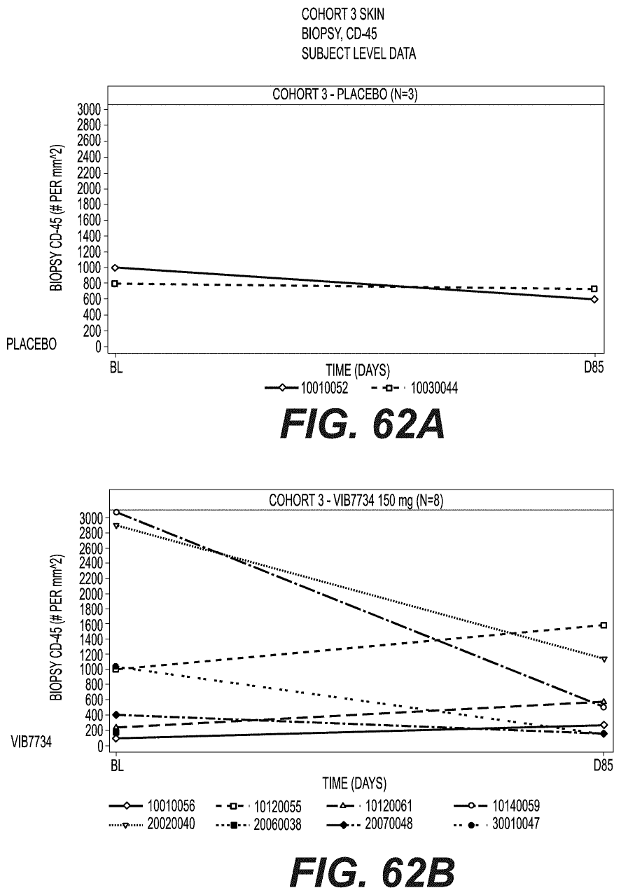

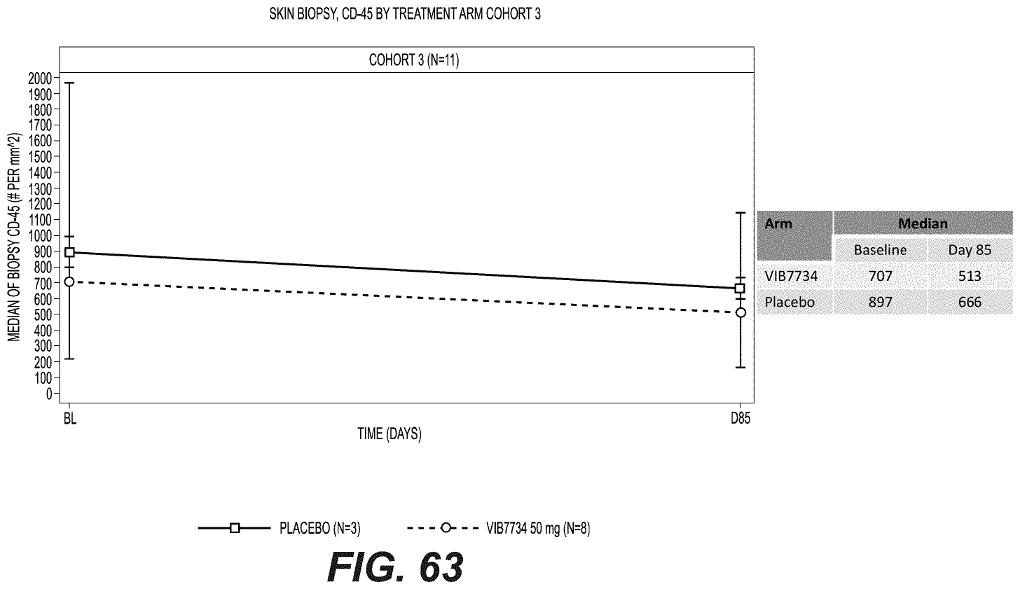

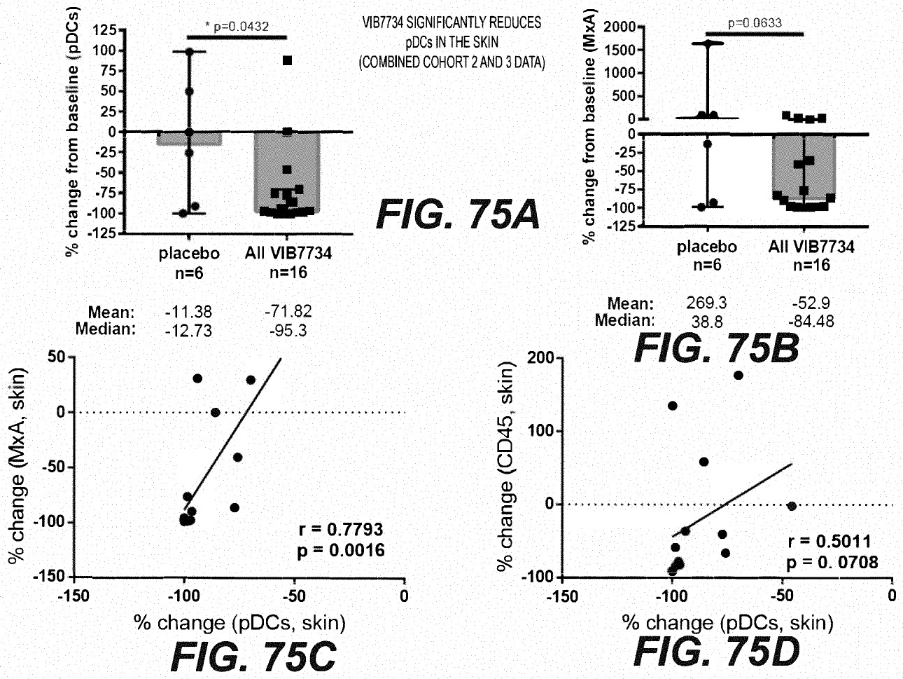

Inflammatory infiltrate (CD45+ cells) is upregulated in skin lesions in patients with CLE. The effect of VIB7734 on inflammatory infiltrate in skin lesions was also determined by assaying levels of CD45+ cells per square mm over time in skin biopsies from subjects in Cohort 2 (FIG. 60) and Cohort 3 (FIG. 62). As shown in FIG. 61, for VIB7734-treated subjects in Cohort 2, the median of skin biopsy CD45 count was reduced from a baseline value of 1119 on Day 1 to 280 on Day 85. In contrast, for placebo-treated Cohort 2 subjects, skin biopsy CD45 count was only reduced from a baseline value of 537 on Day 1 to 492 on Day 85. Also, as shown in FIG. 63, for VIB7734-treated subjects in Cohort 3, the median of skin biopsy CD45 count was reduced from a baseline value of 707 to 513 on Day 85. In contrast, for placebo-treated Cohort 3 subjects, the skin biopsy CD45 count decreased from a baseline value of 897 to 666 on Day 85. Additionally, FIG. 75D shows the correlation between percent change from baseline to day 85 in pDCs and CD45+ cells in combined cohort 2 and cohort 3 VIB7734-treated subjects with ≥2 pDCs/mm² in skin at baseline. In some subjects, the reduction in pDCs following treatment with VIB7734 was accompanied by a dramatic decrease in total CD45+ cells in the skin. Overall, the median reduction in CD45+ cells in all VIB7734-treated subjects was 59.69%, versus −23.97% in the placebo group.

VIB7734 reversibly depletes circulating pDCs with monthly dosing. Additionally, VIB7734 reduces type I IFNGS for the duration of the dosing. VIB7734 reduces also reduces MxA levels in skin and CD45 levels in skin. CLASI scores were improved on treatment with VIB7734 (particularly with the 150 mg dose). No safety issues were identified in subjects with 3 months of dosing with VIB7734. No abnormal or clinically significant ECGs were observed in subjects treated with VIB7734. Further, there were no cases of increase of QT interval by >30 msec in VIB7734-treated subjects.

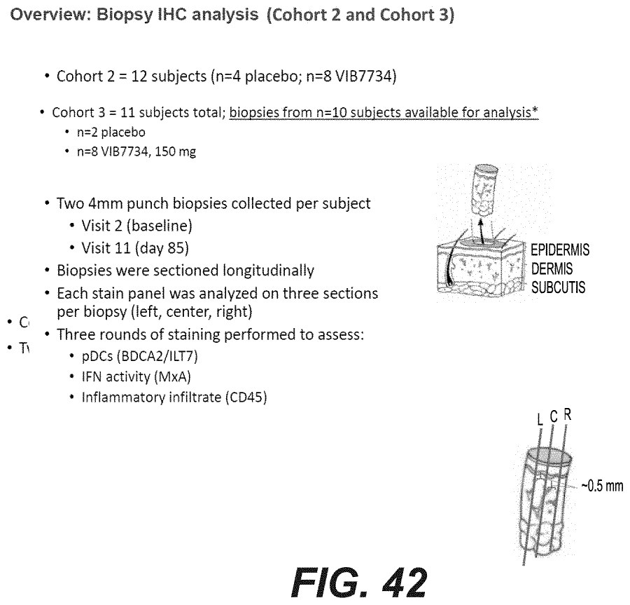

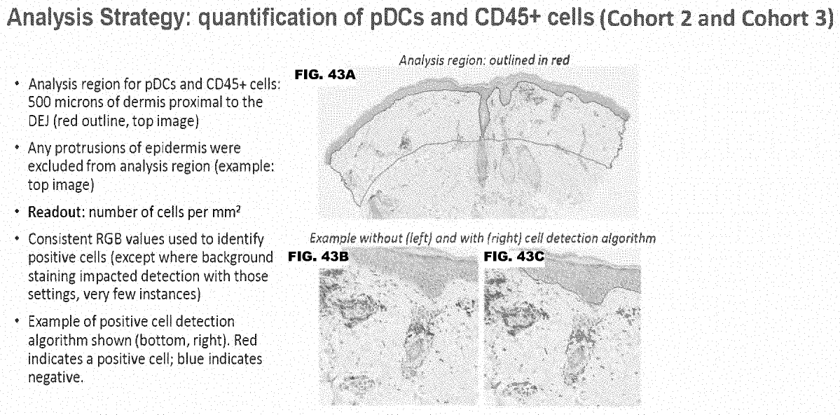

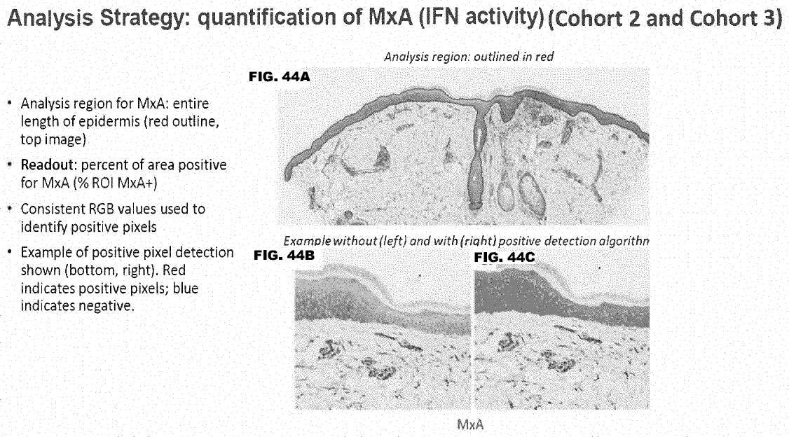

Skin biopsy samples from Cohort 2 and Cohort 3 subjects were also formalin-fixed and paraffin-embedded to enable immunohistochemistry (IHC) and other analyses. FIG. 42 provides an overview of the skin biopsy IHC analysis method used herein. Three rounds of staining was performed to assess pDCs (BDCA+/ILT7+ cells), IFN activity (MxA+ pixels) and inflammatory infiltrate (CD45+ cells). The analysis strategy for quantification of pDCs and CD45+ cells using the skin biopsy IHC analysis method is outlined in FIGS. 43A-43C. The analysis strategy for quantification of MxA for assaying IFN activity using the skin biopsy IHC analysis method is outlined in FIGS. 44A-44C.

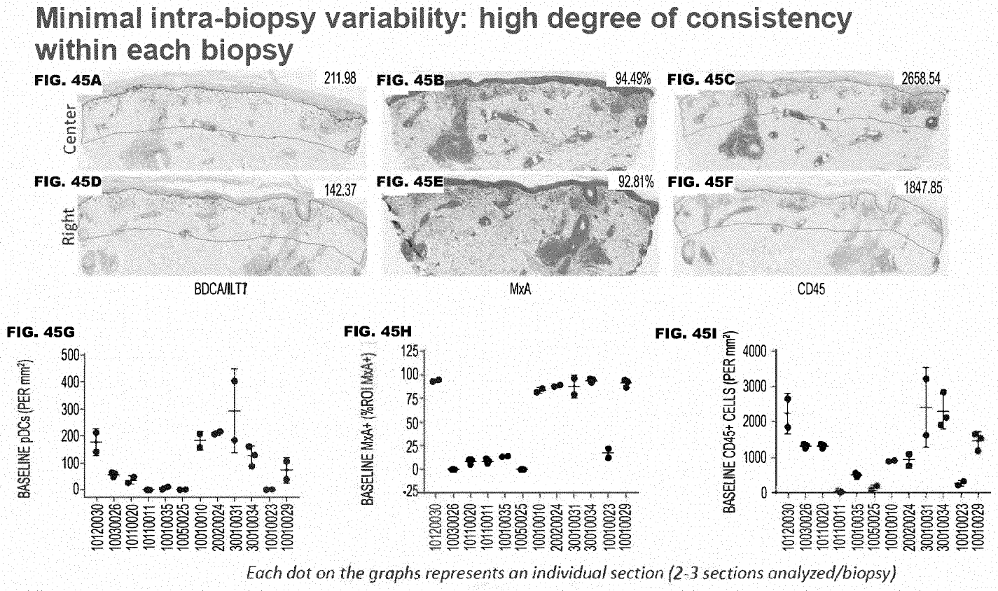

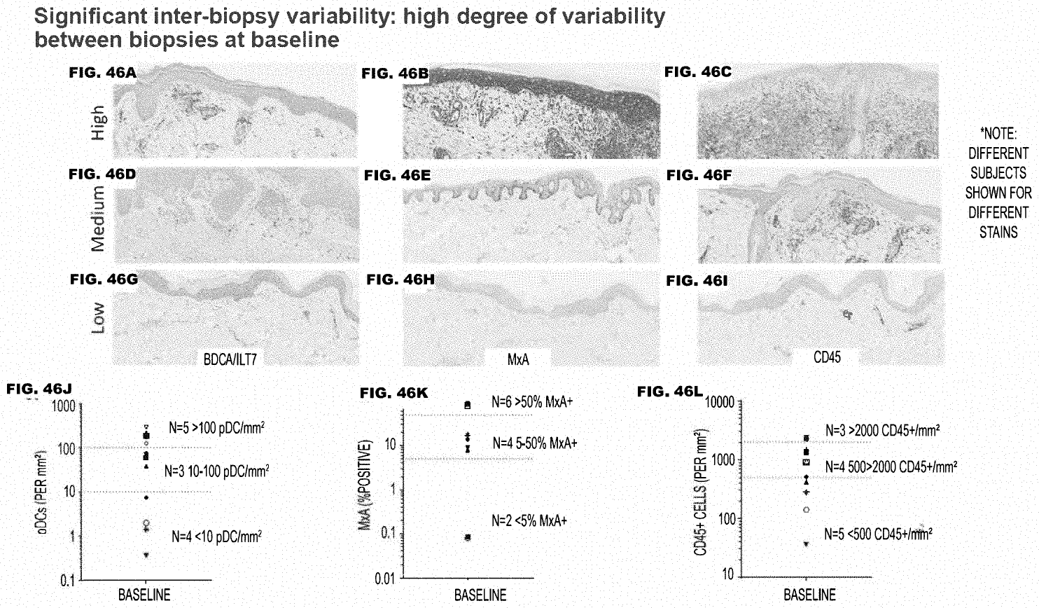

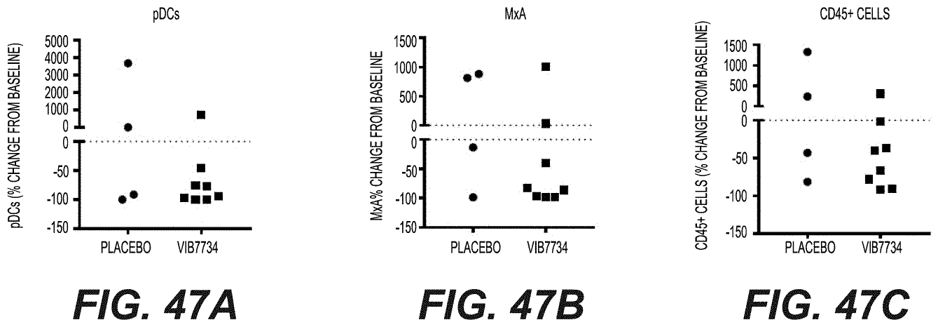

As shown in FIGS. 45A-45I, there was minimal intra-biopsy variability in the baseline numbers of pDCs, MxA+ pixels and CD45+ cells for each subject in Cohort 2. In contrast, there was significant inter-biopsy variability in the baseline numbers of pDCs, MxA+ pixels and CD45+ cells within the subjects in Cohort 2 (FIGS. 46A-46L). FIG. 46J shows the baseline pDCs in skin biopsy from each subject in Cohort 2. The subjects show high baseline pDCs (n=5, >100 pDCs/mm²), medium baseline pDCs (n=3, 10-100 pDCs/mm²), or low baseline pDCs (n=4, <10 pDCs/mm²). FIG. 46K shows baseline MxA+ pixels in skin biopsy from each subject in Cohort 2. The subjects show high baseline MxA+ pixels (n=5, >50% MxA+), medium baseline MxA+ pixels (n=4, 5-50% MxA+), or low baseline MxA+ pixels (n=2, <5% MxA+). FIG. 46L shows baseline CD45+ cells in skin biopsy from each subject in Cohort 2. The subjects show high baseline CD45+ cells (n=3, >2000 CD45+ cells/mm²), medium baseline CD45+ cells (n=4, 500-2000 CD45+ cells/mm²), or low baseline CD45+ cells (n=5, <500 CD45+ cells/mm²). FIG. 47 shows that there was high variability of responses in reductions (measured by the percent change from baseline) in pDCs (FIG. 47A), MxA+ pixels (FIG. 47B), and CD45+ cells (FIG. 47C) in skin biopsies from placebo-treated Cohort 2 subjects. More consistent reductions in pDCs, MxA+ pixels, and CD45+ cells were observed in skin biopsies from VIB7734-treated Cohort 2 subjects.

Thus, there was high variability in the placebo group in Cohort 2 both at baseline and in responses over time in pDC counts and IFN activity in the skin. In contrast, more consistent reductions in pDC counts and IFN activity in skin were observed in the VIB7734-treated group in Cohort 2.

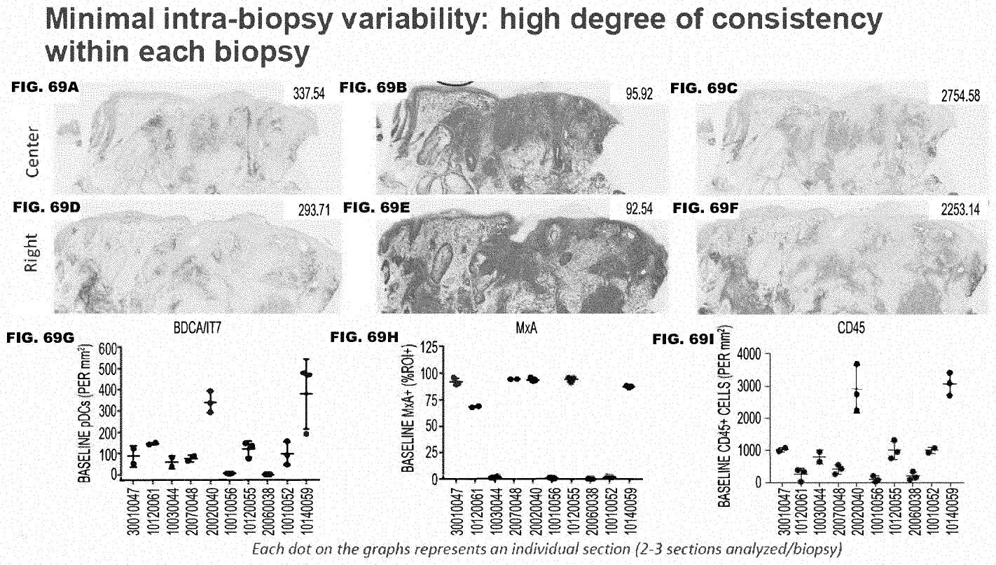

As shown in FIGS. 69A-69I, there was minimal intra-biopsy variability in the baseline numbers of pDCs, MxA+ pixels and CD45+ cells for each subject in Cohort 3. Consistent reductions in pDC counts in the skin were observed in the VIB7734-treated Cohort 3 group.

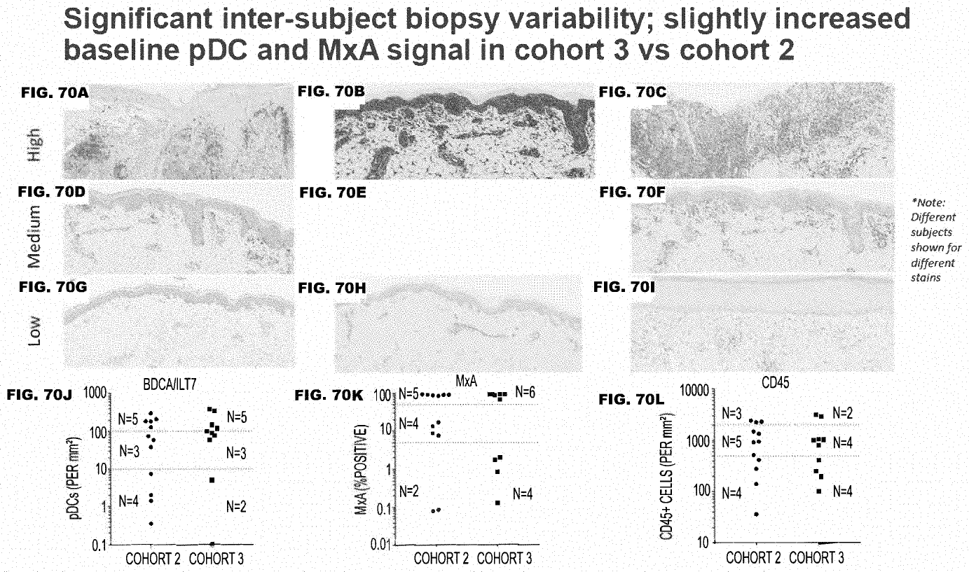

As shown in FIG. 70, there was significant inter-biopsy variability in the baseline numbers of pDCs, MxA+ pixels and CD45+ cells within the subjects in Cohort 3 (FIGS. 70A-70L). A slightly increased baseline pDC and MxA signal was observed in subjects in Cohort 3 compared to subjects in Cohort 2. FIG. 70J shows the baseline pDCs (measured as number of cells per square mm) in skin biopsy from each subject in Cohort 2 and Cohort 3. The subjects in Cohort 2 show high baseline pDCs (n=5, >100 pDCs/mm²), medium baseline pDCs (n=3, 10-100 pDCs/mm²), or low baseline pDCs (n=4, <10 pDCs/mm²). The subjects in Cohort 3 show high baseline pDCs (n=5, >100 pDCs/mm²), medium baseline pDCs (n=3, 10-100 pDCs/mm²), or low baseline pDCs (n=2, <10 pDCs/mm²). FIG. 70K shows the baseline MxA+ pixels (measured as % ROI MxA+) in skin biopsy from each subject in Cohort 2 and Cohort 3. The subjects in Cohort 2 show high baseline MxA+ pixels (n=5, >50% MxA+), medium baseline MxA+ pixels (n=4, 5-50% MxA+), or low baseline MxA+ pixels (n=2, <5% MxA+). The subjects in Cohort 3 show high baseline MxA+ pixels (n=6, >50% MxA+) or low baseline MxA+ pixels (n=4, <5% MxA+). FIG. 70L shows the baseline CD45+ cells (measured as number of cells per square mm) in skin biopsy from each subject in Cohort 2 and Cohort 3. The subjects in Cohort 2 show high baseline CD45+ cells (n=3, >2000 CD45+ cells/mm²), medium baseline CD45+ cells (n=5, 500-2000 CD45+ cells/mm²), or low baseline CD45+ cells (n=4, <500 CD45+ cells/mm²). The subjects in Cohort 3 show high baseline CD45+ cells (n=2, >2000 CD45+ cells/mm²), medium baseline CD45+ cells (n=4, 500-2000 CD45+ cells/mm²), or low baseline CD45+ cells (n=4, <500 CD45+ cells/mm²). Thus, there was slightly higher baseline pDC/IFN activity in skin biopsies in Cohort 3 subjects compared to Cohort 2 subjects.

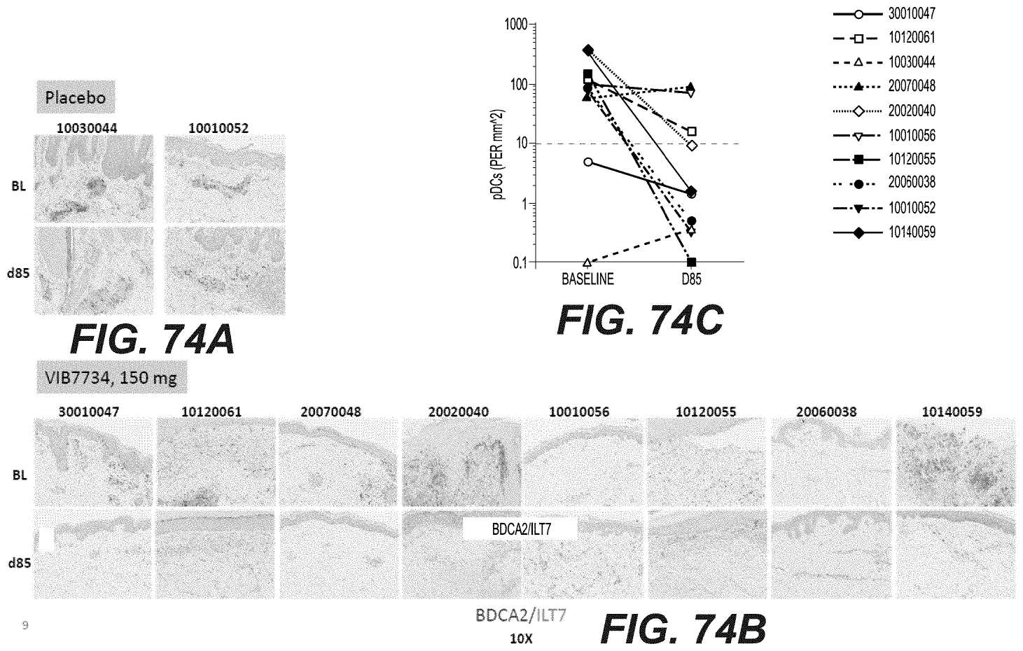

FIG. 74 shows the change in pDCs at Day 85 (d85) from baseline (BL) for each subject in Cohort 3 treated with 150 mg of VIB7734 or a placebo. FIG. 74A shows the change in pDCs (measured as BDCA2+/ILT7+ cells) using the skin biopsy IHC analysis method for subjects in Cohort 3 (n=2; 10030044 and 10010052) treated with a placebo. FIG. 74B shows the change in pDCs (measured as BDCA2+/ILT7+ cells) using the skin biopsy IHC analysis method for subjects in Cohort 3 (n=8; 30010047, 10120061, 20070048, 200020040, 10010056, 10120055, 20060038, and 10140059) treated with 150 mg of VIB7734. FIG. 74C shows the change in pDCs (measured as number of cells per square mm) in skin biopsies of each of the VIB7734-treated Cohort 3 subjects and each of the placebo-treated Cohort 3 subjects at Day 85 compared to baseline.

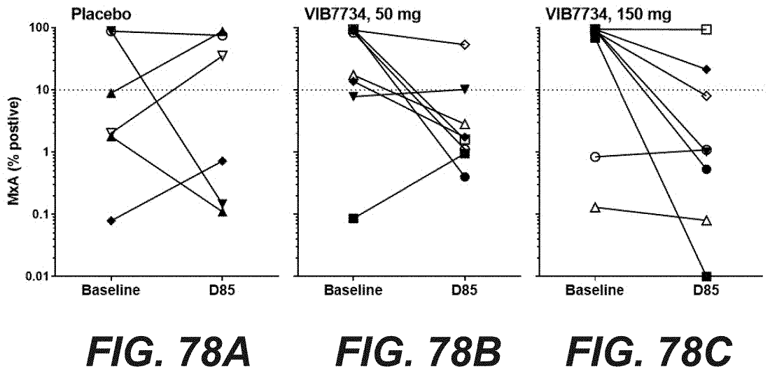

To investigate the contribution of pDCs to local type I IFN activity in the tissue, skin samples from subjects treated with placebo or VIB7734 were also stained for the well-characterized, type I IFN-inducible MxA protein (FIG. 78). Type I IFN activity in the skin of lupus subjects was highly variable at baseline, with a majority of subjects demonstrating high MxA levels in the tissue, while some had little to no MxA signal. In placebo-treated subjects, the change in expression of MxA in the skin over time was also highly variable, with the median area of MxA staining increasing from 5.4% at baseline to 18% at day 85 (FIG. 78A). Treatment with VIB7734 reduced MxA levels in the skin, with a median percent change from baseline of −84.5% for VIB7734-treated participants at day 85 (FIG. 78B-C).

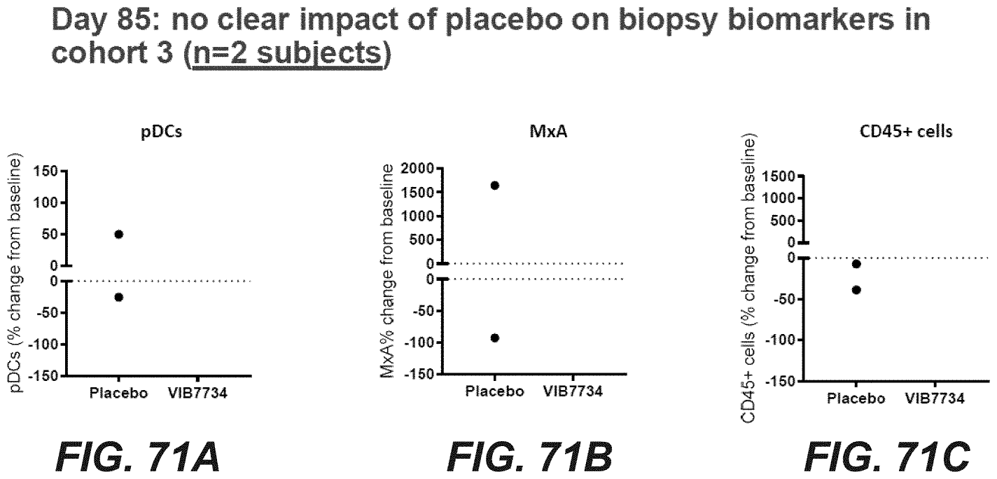

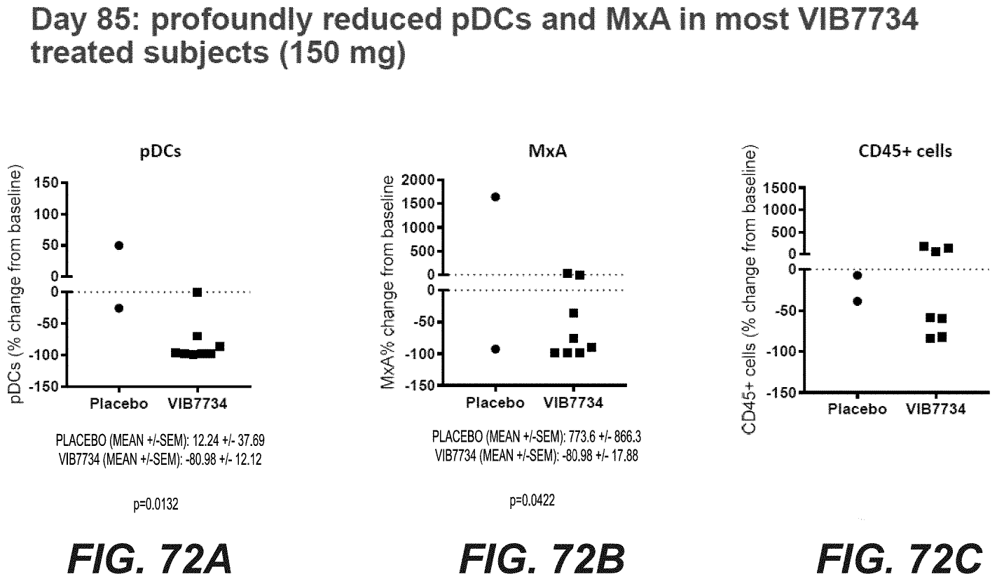

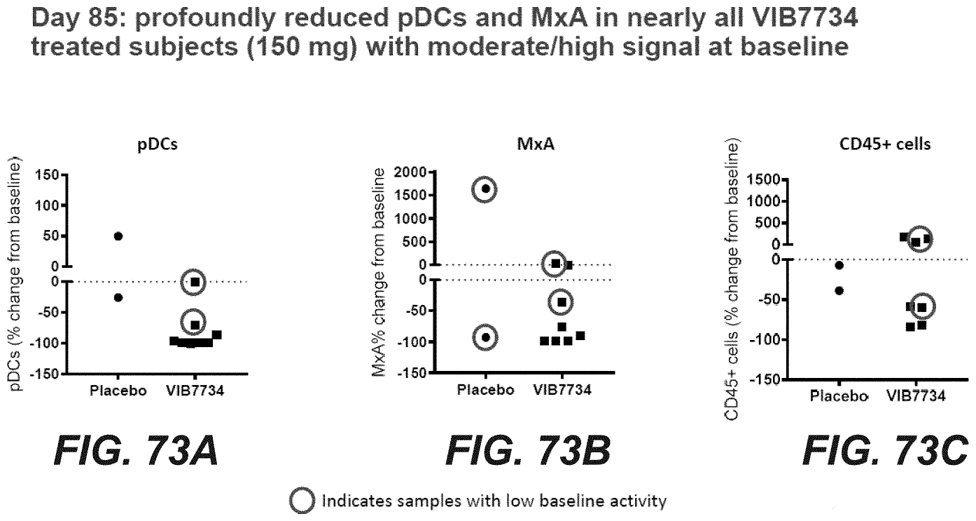

FIG. 71 shows that no clear impact of placebo was observed on skin biopsy markers pDCs (measured as percent change from Day 1 baseline; FIG. 71A), MxA+ pixels (measured as percent change from Day 1 baseline; FIG. 71B) or CD45+ cells (measured as percent change from Day 1 baseline; FIG. 71C) at Day 85 for subjects in Cohort 3. In contrast, as shown in FIGS. 72 and 73, for most subjects in Cohort 3 treated with 150 mg of VIB7734, a profound reduction in pDCs (measured as percent change from Day 1 baseline; FIGS. 72A and 73A) and MxA+ pixels (measured as percent change from Day 1 baseline; FIGS. 72B and 73B) and a reduction in CD45+ cells (measured as percent change from Day 1 baseline; FIGS. 72C and 73C) was observed at Day 85 compared to placebo-treated subjects in Cohort 3. As shown in FIG. 72A, a mean reduction of pDCs of 80.98+/−12.12 (mean+/−SEM) was observed for VIB7734-treated Cohort 3 subjects compared to a mean increase of pDCs of 12.24+/−37.69 (mean+/−SEM) in placebo-treated Cohort 3 subjects. As shown in FIG. 72B, a mean reduction of MxA+ pixels of 58.29+/−17.88 (mean+/−SEM) was observed for VIB7734-treated Cohort 3 subjects compared to a mean increase of MxA+ pixels of 773.6+/−866.33 (mean+/−SEM) in placebo-treated Cohort 3 subjects.

FIG. 75 shows combined data for subjects in Cohort 2 and Cohort 3. VIB7734 significantly reduces pDCs in the skin of subjects in Cohorts 2 and 3 treated with VIB7734 in comparison to subjects in Cohorts 2 and 3 treated with a placebo. As shown in FIG. 75A, at Day 85, the mean and median reductions in pDCs were 11.38% and 12.73%, respectively for placebo-treated subjects in Cohorts 2 and 3 (n=6), compared to mean and median reductions in pDCs of 71.82% and 95.3%, respectively for VIB7734-treated subjects in Cohorts 2 and 3 (n=16). As shown in FIG. 75B, at Day 85, the mean and median increase in MxA+ pixels were 269.3% and 38.8%, respectively for placebo-treated subjects in Cohorts 2 and 3 (n=6), compared to mean and median reductions in pDCs of 52.9% and 84.48%, respectively for VIB7734-treated subjects in Cohorts 2 and 3 (n=16). Correlation analyses were performed to better understand the relationship between changes in pDCs and type I IFN activity in the skin. There was a highly significant correlation between reductions in pDCs and MxA activity within the skin biopsy samples (r=0.7793, FIG. 75C). While dramatic reductions in MxA activity were observed in those subjects with the greatest degree of depletion of tissue pDCs, incomplete/partial pDC depletion often resulted in minimal change in MxA. Thus, combining all placebo and treated subjects (Cohorts 2 and 3) demonstrates significant effect of VIB7734 on tissue biomarkers. These data suggest that pDCs are a critical source of type I IFN in lupus skin and near complete depletion of these cells in the tissue dampens local IFN activity.

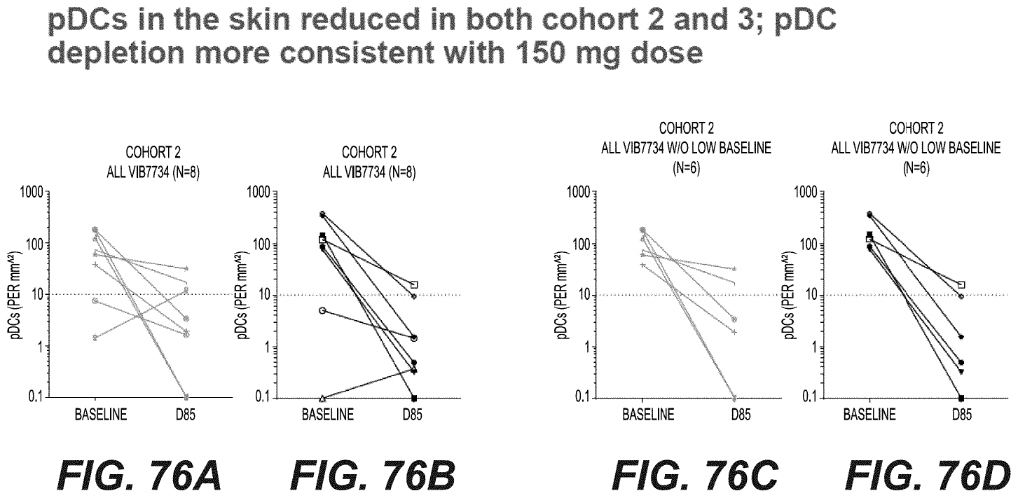

FIG. 76 shows that while pDCs (measured as number of cells per square mm) in the skin were reduced for both Cohort 2 and Cohort 3 subjects, the pDC depletion was more consistent for subjects in Cohort 3. This is true for all VIB7734-treated subjects in Cohort 2 (FIG. 76A) and Cohort 3 (FIG. 76B), as well as for VIB7734-treated Cohort 2 (FIG. 76C) and Cohort 3 (FIG. 76D) subjects without low baseline pDCs or IFN activity in skin biopsy samples.

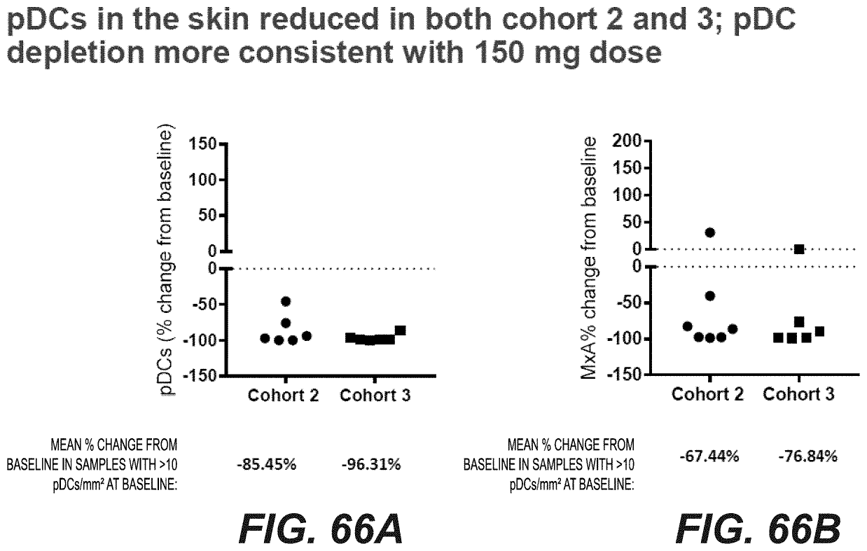

FIG. 66 shows that while pDCs (measured as percent change in number of cells from Day 1 baseline) in the skin were reduced in both Cohort 2 and Cohort 3 subjects treated with VIB7734, the pDC depletion was more consistent for subjects in Cohort 3. As shown in FIG. 66A, the mean percent reduction of pDCs from Day 1 baseline in skin samples with >10 pDCs/mm² at baseline was 96.31% for VIB7734-treated Cohort 3 subjects compared to 85.45% for VIB7734-treated Cohort 2 subjects. Additionally, as shown in FIG. 66B, the mean percent reduction of MxA+ pixels from Day 1 baseline in skin samples with >5% MxA+ at baseline was 76.84% for VIB7734-treated Cohort 3 subjects compared to 67.44% for VIB7734-treated Cohort 2 subjects.

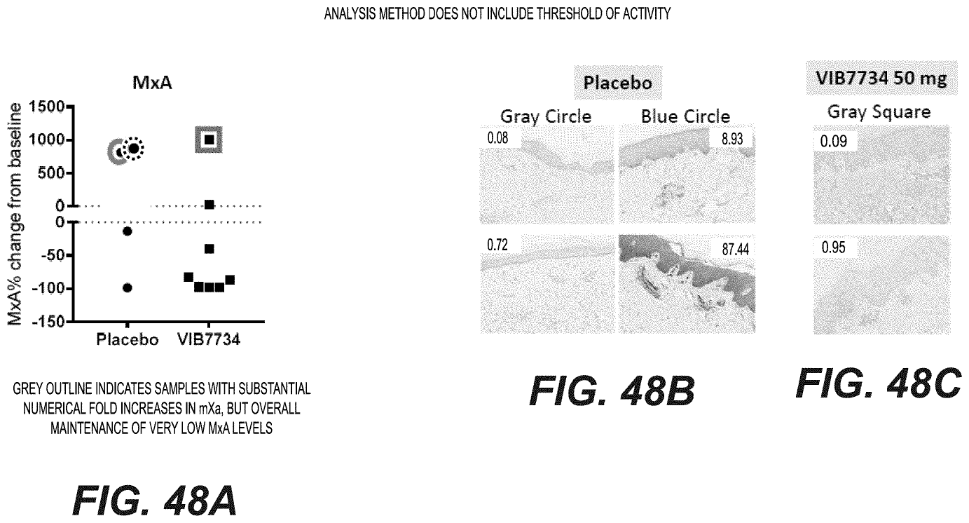

FIG. 48 shows that the skin biopsy IHC analysis method does not include threshold of activity. FIG. 48A: percent change from baseline of MxA in skin biopsies from Cohort 2 subjects treated with a placebo or an ILT7-binding protein used in the methods described herein (VIB7734). Gray outline indicates skin biopsy samples with substantial numerical fold increase in MxA. Overall, however, maintenance of very low levels of MxA was observed in the skin biopsy samples from Cohort 2 subjects. FIG. 48B: IHC performed on a skin biopsies from Cohort 2 subjects following multiple subcutaneous doses (every 4 weeks for 3 doses) of a placebo. FIG. 48C: IHC performed on skin biopsies from Cohort 2 subjects following multiple subcutaneous doses (every 4 weeks for 3 doses) of 50 mg of VIB7734.

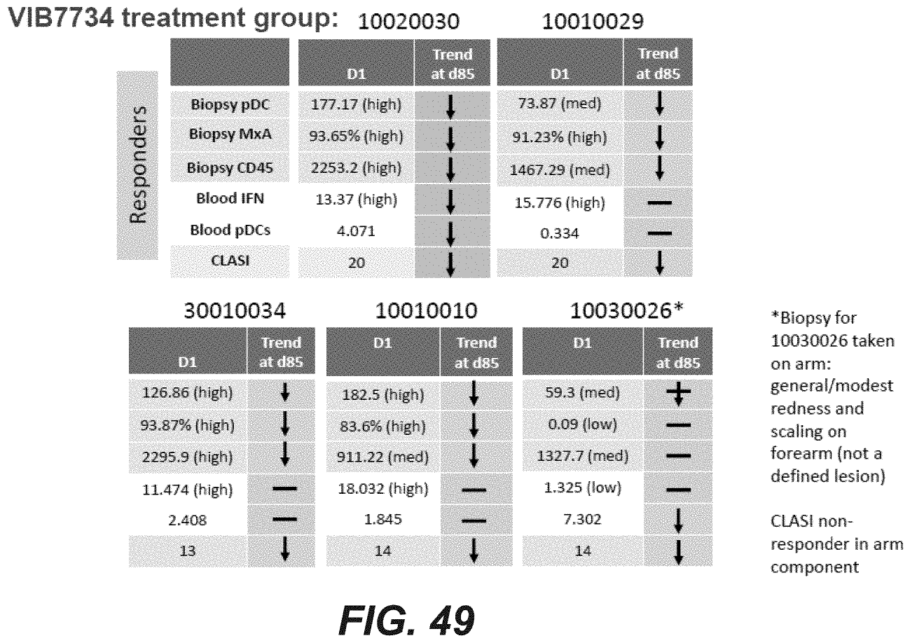

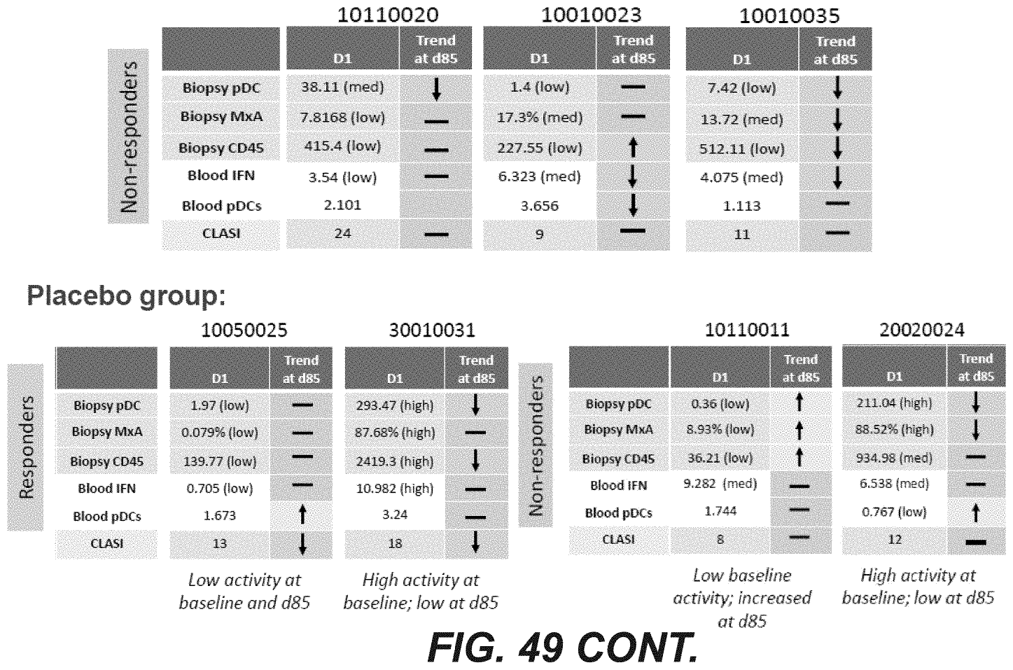

FIG. 49 shows the relationship between high baseline pDC numbers/IFN activity and response to VIB7734 in skin biopsies of Cohort 2 subjects. In the VIB7734 treatment group, high baseline pDC numbers and high IFN activity was observed in skin biopsy in 4 of 5 responders. The non-responders had low baseline pDC or IFN activity in skin biopsy samples. Cohort 2 subjects in the placebo group showed no discernible relationship between pDCs or IFN activity and response. Thus, CLASI responders in the VIB7734 treatment group were largely associated with high pDC/IFN activity in skin and blood IFN at baseline. On the other hand, CLASI non-responders in the VIB7734 treatment group were associated with low/modest pDC/IFN activity in skin at baseline.

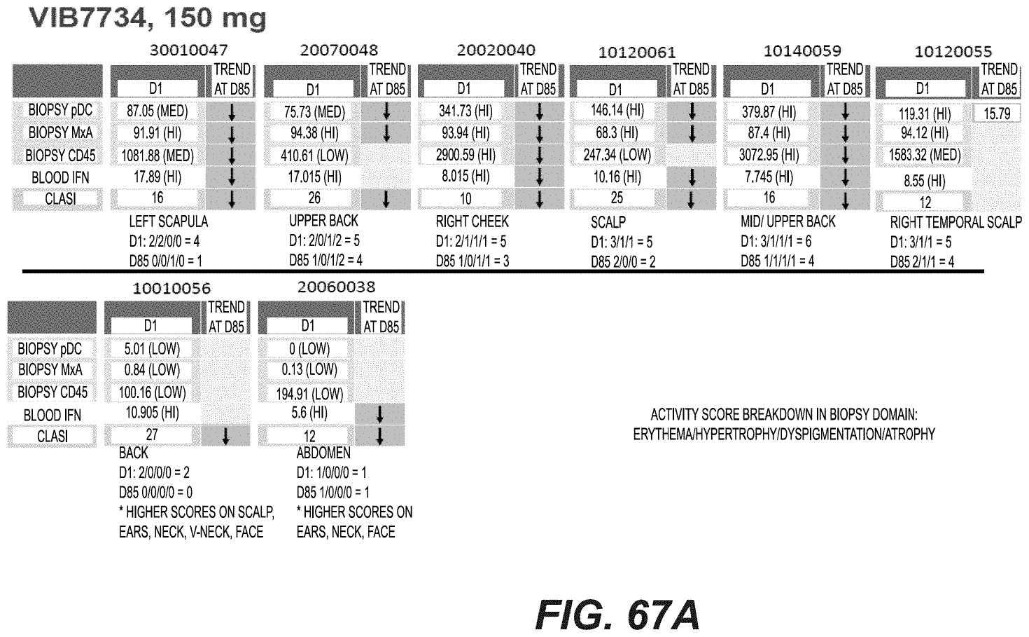

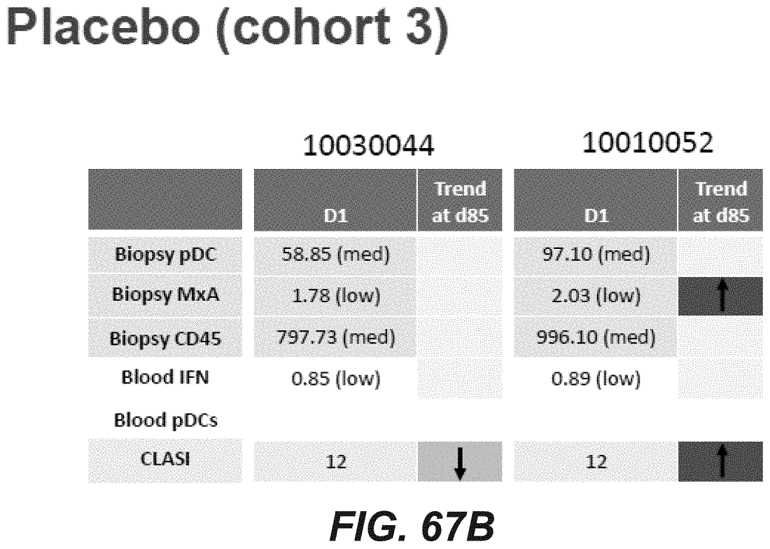

FIG. 67 shows the relationship between high baseline pDC numbers and response to VIB7734 in skin biopsies of Cohort 3 subjects. VIB7734 reduced levels of pDCs in the skin of Cohort 3 subjects (FIG. 67A).

It was further observed that CLASI responders in VIB7734-treated Cohort 3 subjects were largely associated with moderate/high pDC/MxA levels in the skin and high IFN at baseline (note: all VIB7734 treated subjects in Cohort 3 has high baseline blood IFNGS). Depleting pDCs with VIB7734 resulted in a profound reduction in type I IFN activity in CLE skin, demonstrating a fundamental role for these cells in IFNα production in autoimmune tissue.

1. A method for reducing a type I interferon gene signature (IFNGS) in a subject in need thereof, the method comprising administering to the subject a pharmaceutically effective amount of an immunoglobulin-like transcript 7 (ILT7)-binding protein, wherein the ILT7 binding protein is administered to the subject when the type I IFNGS is elevated in the subject relative to the type I IFNGS in a normal subject.

2. The method of claim 1, wherein the type I IFNGS is measured in a test biological sample taken from the subject, and wherein the test biological sample is selected from the group consisting of blood, sputum, saliva, skin biopsy, kidney cells, lung cells, liver cells, heart cells, brain cells, nervous tissue, thyroid cells, eye cells, skeletal muscle cells, cartilage, bone tissue, and cultured cells.

3. The method of claim 2, wherein the test biological sample is blood, the skin biopsy.

4. The method of claim 2, wherein the type I IFNGS is elevated by at least about 4-fold in the test biological sample relative to the type I IFNGS level of the normal subject or a baseline level of a test biological sample of the subject in need thereof as determined by evaluating an mRNA level of a type I interferon gene.

5. (canceled)

6. The method of claim 1, wherein the type I IFNGS is determined by a method comprising determining mRNA levels of at least two genes selected from the group consisting of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18.

7. The method according to claim 6, wherein the type I IFNGS is determined by a method comprising determining mRNA levels all of SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18.

8. (canceled)

9. (canceled)

10. The method of claim 1, wherein the administering is effective in reducing a level of:
i. plasmacytoid dendritic cells (pDCs);
ii. type I IFNGS; and/or
iii. the pDCs and the type I IFNGS.

11. (canceled)

12. (canceled)

13. The method of claim 1, wherein the subject in need has an autoimmune disease.

14. The method of claim 13, wherein the autoimmune disease is selected from the group consisting of systemic lupus erythematosus (SLE), lupus nephritis, cutaneous lupus erythematosus (CLE), Sjögren's syndrome, dermatomyositis, inclusion body myositis, juvenile myositis, polymyositis, systemic sclerosis, diabetes, Hashimoto's disease, autoimmune adrenal insufficiency, anemia, multiple sclerosis, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy.

15. The method of claim 14, wherein the autoimmune disease is Sjögren's syndrome, dermatomyositis, vitiligo, polymyositis, systemic sclerosis, hidradenitis suppurativa, SLE or CLE.

16.-21. (canceled)

22. The method of claim 1, wherein the ILT7-binding protein is an antibody comprising heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

23. The method of claim 22, wherein the ILT7 binding protein is an antibody comprising a variable heavy chain (VH) that is at least 85%, identical to SEQ ID NO:1 and/or a variable light chain (VL) that is at least 85% identical to SEQ ID NO:2.

24. The method of claim 22, wherein the ILT7-binding protein is an antibody comprising a heavy chain variable region (VH) of SEQ ID NO:1 and a light chain variable region (VL) of SEQ ID NO:2.

25. The method of claim 1, wherein the ILT7-binding protein is an antibody that is afucosylated.

26. The method of claim 1, wherein the pharmaceutically effective amount ranges from about 0.1 mg to about 1000 mg.

27. The method of claim 26, wherein the pharmaceutically effective amount is about 1 mg, about 5 mg, about 15 mg, about 50 mg, about 100 mg, or about 150 mg.

28. (canceled)

29. The method of claim 10, comprising the reduced level of type I IFNGS, wherein the reduction is at least about 50% as compared to the level prior to the administration of the ILT7-binding protein.

30.-43. (canceled)

44. A method of treating an autoimmune disorder in a subject in need thereof, the method comprising administering to the subject a pharmaceutically effective amount of an immunoglobulin-like transcript 7 (ILT7)-binding protein, wherein the pharmaceutically effective amount of the ILT7-binding protein is about 1 mg, about 5 mg, about 15 mg, about 50 mg, about 100 mg, or about 150 mg.

45. The method of claim 44, wherein the pharmaceutically effective amount of the ILT7-binding protein is about 50 mg.

46. The method of claim 44, wherein the pharmaceutically effective amount of the ILT7-binding protein is about 150 mg.

47. The method of claim 44, wherein the autoimmune disorder is selected from the group consisting of: systemic lupus erythematosus (SLE), lupus nephritis, cutaneous lupus erythematosus (CLE), Sjögren's syndrome, dermatomyositis, inclusion body myositis, juvenile myositis, polymyositis, systemic sclerosis, diabetes, Hashimoto's disease, autoimmune adrenal insufficiency, anemia, multiple sclerosis, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy.

48.-65. (canceled)

66. A method of treating an autoimmune disorder, the method comprising administering an effective amount of an ILT7 binding protein to a subject in need thereof, wherein the administering is effective in treating the autoimmune disorder as determined by detecting in a sample of the subject:
i. fewer plasmacytoid dendritic cells (pDCs) compared to a sample of the subject before the administering; or
ii. reduced mRNA expression from a gene selected from the group consisting of: SPATS2L, EPSTI1, HERC5, IFI27, IFI44, IFI44L, IFI6, IFIT1, IFIT3, ISG15, LAMP3, LY6E, MX1, OAS1, OAS2, OAS3, PLSCR1, RSAD2, RTP4, SIGLEC1, and USP18,
compared to the mRNA expression level of the gene in a sample of the subject before the administering.

67. The method of claim 66, wherein the ILT7 binding protein is an antibody.

68. The method of claim 67, wherein the antibody comprises heavy chain Complementarity-Determining Regions (HCDRs) HCDR1, HDR2, HCDR3, and light chain Complementarity Determining Regions (LCDRs) LCDR1, LCDR2, and LCDR3 comprising the amino acid sequences of SEQ ID NOs: 3, 4, 5, 6, 7, and 8, respectively.

69. The method of claim 68, wherein the antibody comprises a variable heavy chain (VH) that is at least 85% identical to SEQ ID NO:1 and/or a variable light chain (VL) that is at least 85%, 90%, 95%, 96%, 97%, 98% or 99% identical to SEQ ID NO:2.

70. The method of claim 69, wherein the VH comprises the polypeptide of SEQ ID NO:1, and wherein the VL comprises the polypeptide of SEQ ID NO: 2.

71. The method of claim 66, wherein the autoimmune disorder is selected from the group consisting of: systemic lupus erythematosus (SLE), lupus nephritis, cutaneous lupus erythematosus (CLE), Sjögren's syndrome, dermatomyositis, inclusion body myositis, juvenile myositis, polymyositis, systemic sclerosis, diabetes, Hashimoto's disease, autoimmune adrenal insufficiency, anemia, multiple sclerosis, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy.
